(19) United States
(12) Patent Application Publication
(10) Pub. No.: US 2026/0335894 A1
(43) Pub. Date: Oct. 29, 2026

(54) MICRO-OBJECT DISPLACEMENT WITHIN A MICROFLUIDIC ENVIRONMENT

(71) Applicant: Bruker Spatial Biology, Inc., Seattle, WA (US)

(72) Inventor: Hector D. NEIRA-QUINTERO, Emeryville, CA (US)

(21) Appl. No.: 19/118,193

(22) PCT Filed: Oct. 2, 2023
(86) PCT No.: PCT/US2023/075731

§ 371 Date: Apr. 3, 2025

Related U.S. Application Data
(60) Provisional application No. 63/378,183, filed on Oct. 3, 2022.

(51) Int. Cl. B01L 3/00

(52) U.S. Cl. CPC B01L 3/502761; B01L 3/50273; B01L 2200/0647; B01L 2400/0478

(57) ABSTRACT

The present disclosure relates to systems, methods, kits, and computer readable media for assaying and controlling micro-objects in a microfluidic device. In situ-generated hydrogel barriers within a microfluidic chamber, sub-dividing the chamber into areas where forces suitable for displacing a micro-object from a cell may be generated without interference to the cell itself.

### CROSS-REFERENCE TO RELATED APPLICATIONS

This application claims the benefit of priority to U.S. Provisional Patent Application Ser. No. 63/378,183, filed Oct. 3, 2022, hereby incorporated by reference in its entirety.

### BACKGROUND

Microfluidic devices allow researchers to manipulate and categorize micro-objects such as biological cells. The present disclosure relates to systems, methods, kits, and computer-readable media for moving micro-objects in a microfluidic device.

### SUMMARY

In a first aspect, provided herein are methods for displacing a micro-object from a chamber of a microfluidic device. In some embodiments, a method for displacing a micro-object from a chamber of a microfluidic device comprises: a) providing the microfluidic device, wherein the microfluidic device comprises a microfluidic circuit comprising a substrate, a flow region and the chamber, wherein the chamber comprises an opening to the flow region; wherein the micro-object is disposed within the chamber; b) forming an in situ-generated piston within a region of the chamber distal to the micro-object, wherein the in situ-generated piston defines a target region within the chamber distal to the in situ-generated piston; and c) illuminating the target region to generate a displacement force thereby displacing the micro-object from the chamber.

In some embodiments, a micro-object further comprises a plurality of micro-objects. In some embodiments, before forming an in situ-generated piston, the method further comprises moving a portion of the plurality of micro-objects away from a region of the chamber most distal from the opening of the chamber. In some embodiments, moving a portion of the plurality of micro-objects further comprises moving the portion of the plurality of micro-objects toward the opening of the chamber. In some embodiments, a target region defined by the in situ-generated piston is substantially absent of the micro-object. In some embodiments, providing the microfluidic device further comprises disposing the micro-object within the chamber. In some embodiments, a method further comprises culturing the micro-object within the chamber.

In some embodiments, illuminating the target region comprises illuminating the target region with a laser. In some embodiments, illuminating the target region results in generating a bubble within the target region, wherein the bubble generates the displacement force. In some embodiments, the displacement force pushes the in situ-generated piston away from its original position. In some embodiments, the displacement force pushes the in situ-generated piston toward the opening of the chamber. In some embodiments, illuminating the target region comprises directing illumination towards the substrate; a microfluidic circuit material of a wall; or a thermal target. In some embodiments, the thermal target comprises a metal deposit, a pattern of metal deposits, or microstructures patterned on a surface. In some embodiments, illuminating the target region comprises illuminating with illumination having incident power in the range of about 1 mW to about 1000 mW.

In some embodiments, the in situ-generated piston has a porosity that substantially prevents the micro-object from crossing through the in situ-generated piston. In some embodiments, displacing the micro-object comprises exporting the micro-object into the flow region, and optionally, exporting the micro-object from the microfluidic device. In some embodiments, exporting the micro-object from the microfluidic device further comprises flowing media within the flow region. In some embodiments, the flow region comprises a microfluidic channel, and the opening of the chamber is proximal to the microfluidic channel and oriented substantially parallel to a direction of flow of a fluidic medium in the microfluidic channel (e.g., when the fluidic medium is flowing in the microfluidic channel). In some embodiments, the chamber comprises an isolation region and a connection region fluidically connecting the isolation region to the flow region; and wherein the connection region comprises the opening to the flow region. In some embodiments, the micro-object is disposed within the isolation region. In some embodiments, the target region is within the isolation region. In some embodiments, the in situ-generated piston comprises a first solidified polymer network.

In some embodiments, methods described herein further comprise forming an in situ-generated guide element comprising a second solidified polymer network within an area proximal to the opening of the chamber. In some embodiments, the area proximal to the opening of the chamber is within the chamber. In some embodiments, the in situ-generated guide element comprises at least one gap configured to permit displacing the micro-object and impede re-entry of the micro-object into the chamber from the flow region. In some embodiments, the first solidified polymer network (e.g., a piston) and the second solidified polymer network (e.g., a guide element) independently comprises a synthetic polymer, a modified synthetic polymer, or a biological polymer. In some embodiments, the first solidified polymer network and the second solidified polymer network independently comprises at least one of a polyethylene glycol, modified polyethylene glycol, polyglycolic acid (PGA), modified polyglycolic acid, polyacrylamide (PAM), modified polyacrylamide, poly-N-isopropylacrylamide (PNIPAm), modified poly-N-isopropylacrylamide, polyvinyl alcohol (PVA), modified polyvinyl alcohol, polyacrylic acid (PAA), modified polyacrylic acid, fibronectin, modified fibronectin, collagen, modified collagen, laminin, modified laminin, polysaccharide, modified polysaccharide, or a co-polymer in any combination. In some embodiments, the solidified polymer network comprises a polyethylene glycol acrylamide polymer. In some embodiments, the polyethylene glycol acrylamide polymer comprises a linear polyethylene glycol diacrylamide polymer, two arm polyethylene glycol acrylamide polymer, a star polyethylene glycol acrylamide polymer, or a mixture of any combination thereof. In some embodiments, the polyethylene glycol acrylamide polymer is other than a linear polyethylene glycol acrylamide polymer, all of the termini comprise an acrylamide moiety. In some embodiments, the polyethylene glycol acrylamide polymer is other than a linear polyethylene glycol acrylamide polymer, less than all of the termini comprise an acrylamide moiety.

In some embodiments, forming the in situ-generated piston and/or the in situ-generated guide element comprises flowing a first fluidic medium comprising a flowable polymer solution into the flow region of the microfluidic device; and allowing the flowable polymer solution to diffuse into the chamber. In some embodiments, forming the in situ-generated piston and/or the in situ-generated guide element further comprises solidifying the flowable polymer solution within the chamber using photopatterning. In some embodiments, the micro-object is a biological cell (e.g. an eukaryotic cell or a prokaryotic cell) or a bead. In some embodiments, the biological cell is an animal cell, a plant cell, or a bacteria cell.

In an additional aspect, provided herein are kits for displacing a micro-object from a chamber of a microfluidic device. In some embodiments, a kit for displacing a micro-object from a chamber of a microfluidic comprises: a) a flowable polymer configured to be controllably activated to form an in situ-generated barrier comprising a solidified polymer network; and b) an inhibitor.

In some embodiments, a kit further comprises an initiator. In some embodiments, the initiator is a photactivatable initiator. In some embodiments, a kit further comprises a microfluidic device comprising a microfluidic circuit comprising a flow region and a chamber, wherein the chamber comprises an opening to the flow region. In some embodiments, the flow region comprises a microfluidic channel, and the opening of the chamber is proximal to the microfluidic channel and oriented substantially parallel to a flow of a fluidic medium in the microfluidic channel, when the fluidic medium is flowing in the microfluidic channel. In some embodiments, the chamber comprises an isolation region and a connection region fluidically connecting the isolation region to the flow region; and wherein the connection region comprises the opening to the flow region. In some embodiments, the microfluidic device comprises a plurality of chambers. In some embodiments, the microfluidic device comprises a substrate configured to generate dielectrophoresis (DEP) forces within the microfluidic circuit. In some embodiments, the solidified polymer network comprises a synthetic polymer, a modified synthetic polymer, or a biological polymer. In some embodiments, the solidified polymer network comprises at least one of a polyethylene glycol, modified polyethylene glycol, polyglycolic acid (PGA), modified polyglycolic acid, polyacrylamide (PAM), modified polyacrylamide, poly-N-isopropylacrylamide (PNIPAm), modified poly-N-isopropylacrylamide, polyvinyl alcohol (PVA), modified polyvinyl alcohol, polyacrylic acid (PAA), modified polyacrylic acid, fibronectin, modified fibronectin, collagen, modified collagen, laminin, modified laminin, polysaccharide, modified polysaccharide, or a co-polymer in any combination. In some embodiments, the solidified polymer network comprises a polyethylene glycol acrylamide polymer. In some embodiments, the polyethylene glycol acrylamide polymer comprises a linear polyethylene glycol diacrylamide polymer, two arm polyethylene glycol acrylamide polymer, a star polyethylene glycol acrylamide polymer, or a mixture of any combination thereof. In some embodiments, the polyethylene glycol acrylamide polymer is other than a linear polyethylene glycol acrylamide polymer, all of the termini comprise an acrylamide moiety. In some embodiments, the polyethylene glycol acrylamide polymer is other than a linear polyethylene glycol acrylamide polymer, less than all of the termini comprise an acrylamide moiety.

### BRIEF DESCRIPTION OF THE DRAWINGS

FIG. 1A illustrates a microfluidic device and a system with associated control equipment according to some embodiments of the disclosure.

FIG. 1B illustrates a microfluidic device with sequestration pens according to an embodiment of the disclosure.

FIGS. 2A to 2B illustrate a microfluidic device having sequestration pens according to some embodiments of the disclosure.

FIG. 2C illustrates a sequestration pen of a microfluidic device according to some embodiments of the disclosure.

FIG. 3 illustrates a sequestration pen of a microfluidic device according to some embodiments of the disclosure.

FIGS. 4A to 4B illustrate electrokinetic features of a microfluidic device according to some embodiments of the disclosure.

FIG. 5A illustrates a system for use with a microfluidic device and associated control equipment according to some embodiments of the disclosure.

FIG. 5B illustrates an imaging device according to some embodiments of the disclosure.

FIG. 6A is a graphical representation of a sequestration pen of a microfluidic device comprising in situ-generated piston and in situ-generated guide element, and regions formed following creation of the piston, according to some embodiments of the disclosure.

FIG. 6B is a photographic representations of sequestration pens of a microfluidic device comprising in situ-generated pistons and in situ-generated guide elements, and regions formed following creation of the piston, according to some embodiments of the disclosure.

FIG. 7 is a series of photographic representations of a time lapsed displacement process comprising use of in situ-generated pistons and in situ-generated guide elements according to some embodiments of the disclosure.

FIG. 8 is a block diagram for displacing a micro-object out of a sequestration pen of a microfluidic device.

FIG. 9 is another block diagram for displacing a micro-object out of a sequestration pen of a microfluidic device.

### DETAILED DESCRIPTION

Some of the techniques described herein include forming in situ-generated barriers to facilitate displacement of micro-objects within a microfluidic device. For example, a microfluidic device can be used for culturing and/or assaying biological cells isolated within separate sequestration pens. To isolate the cells within the separate sequestration pens, dielectrophoresis (DEP) (e.g., an optically actuated dielectrophoretic force (OEP)), might be employed to provide force to move the biological cells into sequestration pens. After culturing and/or assaying, the biological cells should be displaced from the sequestration pens and moved into a microfluidic channel. However, some biological cells might adhere to the surface of a sequestration pen. The force provided by DEP might not be strong enough to easily or successfully remove the biological cells from the sequestration pen.

As described herein, an in situ-generated structure can be formed to divide a sequestration pen into a target region and a culture region. A biological cell can then be positioned, using DEP forces, within the culture region on one side of the in situ-generated structure. A laser can then illuminate the target region on the other side of the in situ-generated structure to heat the fluidic medium within the target region. This heating causes a bubble to form and manipulate the situ-generated structure such that a displacement force can move the biological cell out of the sequestration pen. This displacement force can be stronger than the DEP force and, therefore, can displace biological cells that might be adhered to the surface and difficult to manipulate using DEP forces. Because the in situ-generated structure isolates the biological cell in the culture region away from the laser illumination in the target region, the biological cell is not harmed by the direct application of the displacement force provided by the bubble. Thus, the biological cell may be displaced out of the sequestration pen without being harmed by the displacement force.

In more detail, this specification describes exemplary embodiments and applications of the disclosure. The disclosure, however, is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion. In addition, as the terms “on,” “attached to,” “connected to,” “coupled to,” or similar words are used herein, one element (e.g., a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. Also, unless the context dictates otherwise, directions (e.g., above, below, top, bottom, side, up, down, under, over, upper, lower, horizontal, vertical, “x,” “y,” “z,” etc.), if provided, are relative and provided solely by way of example and for ease of illustration and discussion and not by way of limitation. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

Where dimensions of microfluidic features are described as having a width or an area, the dimension typically is described relative to an x-axial and/or y-axial dimension, both of which lie within a plane that is parallel to the substrate and/or cover of the microfluidic device. The height of a microfluidic feature may be described relative to a z-axial direction, which is perpendicular to a plane that is parallel to the substrate and/or cover of the microfluidic device. In some instances, a cross sectional area of a microfluidic feature, such as a channel or a passageway, may be in reference to a x-axial/z-axial, a y-axial/z-axial, or an x-axial/y-axial area.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “ones” means more than one. As used herein, the term “plurality” can be 2, 3, 4, 5, 6, 7, 8, 9, 10, or more.

As used herein: μm means micrometer, μm³ means cubic micrometer, pL means picoliter, nL means nanoliter, and μL (or uL) means microliter.

As used herein, “air” refers to the composition of gases predominating in the atmosphere of the earth. The four most plentiful gases are nitrogen (typically present at a concentration of about 78% by volume, e.g., in a range from about 70-80%), oxygen (typically present at about 20.95% by volume at sea level, e.g. in a range from about 10% to about 25%), argon (typically present at about 1.0% by volume, e.g. in a range from about 0.1% to about 3%), and carbon dioxide (typically present at about 0.04%, e.g., in a range from about 0.01% to about 0.07%). Air may have other trace gases such as methane, nitrous oxide or ozone, trace pollutants and organic materials such as pollen, diesel particulates and the like. Air may include water vapor (typically present at about 0.25% or may be present in a range from about 10 ppm to about 5% by volume). Air may be provided for use in culturing experiments as a filtered, controlled composition and may be conditioned as described herein.

As used herein, the term “disposed” encompasses within its meaning “located.”

As used herein, a “microfluidic device” or “microfluidic apparatus” is a device that includes one or more discrete microfluidic circuits configured to hold a fluid, each microfluidic circuit comprised of fluidically interconnected circuit elements, including but not limited to region(s), flow path(s), channel(s), chamber(s), and/or pen(s), and at least one port configured to allow the fluid (and, optionally, micro-objects suspended in the fluid) to flow into and/or out of the microfluidic device. Typically, a microfluidic circuit of a microfluidic device will include a flow region, which may include a microfluidic channel, and at least one chamber, and will hold a volume of fluid of less than about 1 mL, e.g., less than about 750, 500, 250, 200, 150, 100, 75, 50, 25, 20, 15, 10, 9, 8, 7, 6, 5, 4, 3, or 2 microliters. In certain embodiments, the microfluidic circuit holds about 1-2, 1-3, 1-4, 1-5, 2-5, 2-8, 2-10, 2-12, 2-15, 2-20, 5-20, 5-30, 5-40, 5-50, 10-50, 10-75, 10-100, 20-100, 20-150, 20-200, 50-200, 50-250, or 50-300 μL. The microfluidic circuit may be configured to have a first end fluidically connected with a first port (e.g., an inlet) in the microfluidic device and a second end fluidically connected with a second port (e.g., an outlet) in the microfluidic device.

As used herein, a “nanofluidic device” or “nanofluidic apparatus” is a type of microfluidic device having a microfluidic circuit that contains at least one circuit element configured to hold a volume of fluid of less than about 1 μL, e.g., less than about 750, 500, 250, 200, 150, 100, 75, 50, 25, 20, 15, 10, 9, 8, 7, 6, 5, 4, 3, 2, 1 nL or less. A nanofluidic device may comprise a plurality of circuit elements (e.g., at least 2, 3, 4, 5, 6, 7, 8, 9, 10, 15, 20, 25, 50, 75, 100, 150, 200, 250, 300, 400, 500, 600, 700, 800, 900, 1000, 1500, 2000, 2500, 3000, 3500, 4000, 4500, 5000, 6000, 7000, 8000, 9000, 10,000, or more). In certain embodiments, one or more (e.g., all) of the at least one circuit elements is configured to hold a volume of fluid of about 100 μL to 1 nL, 100 μL to 2 nL, 100 μL to 5 nL, 250 μL to 2 nL, 250 pL to 5 nL, 250 μL to 10 nL, 500 μL to 5 nL, 500 μL to 10 nL, 500 μL to 15 nL, 750 μL to 10 nL, 750 μL to 15 nL, 750 μL to 20 nL, 1 to 10 nL, 1 to 15 nL, 1 to 20 nL, 1 to 25 nL, or 1 to 50 nL. In other embodiments, one or more (e.g., all) of the at least one circuit elements are configured to hold a volume of fluid of about 20 nL to 200 nL, 100 to 200 nL, 100 to 300 nL, 100 to 400 nL, 100 to 500 nL, 200 to 300 nL, 200 to 400 nL, 200 to 500 nL, 200 to 600 nL, 200 to 700 nL, 250 to 400 nL, 250 to 500 nL, 250 to 600 nL, or 250 to 750 nL.

A microfluidic device or a nanofluidic device may be referred to herein as a “microfluidic chip” or a “chip”; or “nanofluidic chip” or “chip”.

A “microfluidic channel” or “flow channel” as used herein refers to flow region of a microfluidic device having a length that is significantly longer than both the horizontal and vertical dimensions. For example, the flow channel can be at least 5 times the length of either the horizontal or vertical dimension, e.g., at least 10 times the length, at least 25 times the length, at least 100 times the length, at least 200 times the length, at least 500 times the length, at least 1,000 times the length, at least 5,000 times the length, or longer. In some embodiments, the length of a flow channel is about 100,000 microns to about 500,000 microns, including any value therebetween. In some embodiments, the horizontal dimension is about 100 microns to about 1000 microns (e.g., about 150 to about 500 microns) and the vertical dimension is about 25 microns to about 200 microns, (e.g., from about 40 to about 150 microns). It is noted that a flow channel may have a variety of different spatial configurations in a microfluidic device, and thus is not restricted to a perfectly linear element. For example, a flow channel may be, or include one or more sections having, the following configurations: curve, bend, spiral, incline, decline, fork (e.g., multiple different flow paths), and any combination thereof. In addition, a flow channel may have different cross-sectional areas along its path, widening and constricting to provide a desired fluid flow therein. The flow channel may include valves, and the valves may be of any type known in the art of microfluidics. Examples of microfluidic channels that include valves are disclosed in U.S. Pat. Nos. 6,408,878 and 9,227,200, each of which is herein incorporated by reference in its entirety.

As used herein, the term “transparent” refers to a material which allows visible light to pass through without substantially altering the light as is passes through.

As used herein, “brightfield” illumination and/or image refers to white light illumination of the microfluidic field of view from a broad-spectrum light source, where contrast is formed by absorbance of light by objects in the field of view.

As used herein, “structured light” is projected light that is modulated to provide one or more illumination effects. A first illumination effect may be projected light illuminating a portion of a surface of a device without illuminating (or at least minimizing illumination of) an adjacent portion of the surface, e.g., a projected light pattern, as described more fully below, used to activate DEP forces within a DEP substrate. When using structured light patterns to activate DEP forces, the intensity, e.g., variation in duty cycle of a structured light modulator such as a DMD, may be used to change the optical power applied to the light activated DEP actuators, and thus change DEP force without changing the nominal voltage or frequency. Another illumination effect that may be produced by structured light includes projected light that may be corrected for surface irregularities and for irregularities associated with the light projection itself, e.g., fall-off at the edge of an illuminated field. Structured light is typically generated by a structured light modulator, such as a digital mirror device (DMD), a microshutter array system (MSA), a liquid crystal display (LCD), or the like. Illumination of a small area of the surface, e.g., a selected area of interest, with structured light improves the signal-to-noise-ratio (SNR), as illumination of only the selected area of interest reduces stray/scattered light, thereby lowering the dark level of the image. An important aspect of structured light is that it may be changed quickly over time. A light pattern from the structured light modulator, e.g., DMD, may be used to autofocus on difficult targets such as clean mirrors or surfaces that are far out of focus. Using a clean mirror, a number of self-test features may be replicated such as measurement of modulation transfer function and field curvature/tilt, without requiring a more expensive Shack-Hartmann sensor. In another use of structured light patterns, spatial power distribution may be measured at the sample surface with a simple power meter, in place of a camera. Structured light patterns may also be used as a reference feature for optical module/system component alignment as well used as a manual readout for manual focus. Another illumination effect made possible by use of structured light patterns is selective curing, e.g., solidification of hydrogels within the microfluidic device.

As used herein, the term “micro-object” refers generally to any microscopic object that may be isolated and/or manipulated in accordance with the present disclosure. Non-limiting examples of micro-objects include: inanimate micro-objects such as microparticles; microbeads (e.g., polystyrene beads, glass beads, amorphous solid substrates, Luminex™ beads, or the like); magnetic beads; microrods; microwires; quantum dots, and the like; biological micro-objects such as cells; biological organelles; vesicles, or complexes; synthetic vesicles; liposomes (e.g., synthetic or derived from membrane preparations); lipid nanorafts, and the like; or a combination of inanimate micro-objects and biological micro-objects (e.g., microbeads attached to cells, liposome-coated micro-beads, liposome-coated magnetic beads, or the like). Beads may include moieties/molecules covalently or non-covalently attached, such as fluorescent labels, proteins (including receptor molecules), carbohydrates, antigens, small molecule signaling moieties, or other chemical/biological species capable of use in an assay. In some variations, beads/solid substrates including moieties/molecules may be capture beads, e.g., configured to bind molecules including small molecules, peptides, proteins or nucleic acids present in proximity either selectively or nonselectively. In one nonlimiting example, a capture bead may include a nucleic acid sequence configured to bind nucleic acids having a specific nucleic acid sequence or the nucleic acid sequence of the capture bead may be configured to bind a set of nucleic acids having related nucleic acid sequences. Type of binding may be understood to be selective. Capture beads containing moieties/molecules may bind nonselectively when binding of structurally different but physico-chemically similar molecules is performed, for example, size exclusion beads or zeolites configured to capture molecules of selected size or charge. Lipid nanorafts have been described, for example, in Ritchie et al. (2009) “Reconstitution of Membrane Proteins in Phospholipid Bilayer Nanodiscs,” Methods Enzymol., 464:211-231.

As used herein, the term “cell” is used interchangeably with the term “biological cell.” Non-limiting examples of biological cells include eukaryotic cells, plant cells, animal cells, such as mammalian cells, reptilian cells, avian cells, fish cells, or the like, prokaryotic cells, bacterial cells, fungal cells, protozoan cells, or the like, cells dissociated from a tissue, such as muscle, cartilage, fat, skin, liver, lung, neural tissue, and the like, immunological cells, such as T cells, B cells, natural killer cells, macrophages, and the like, embryos (e.g., zygotes), oocytes, ova, sperm cells, hybridomas, cultured cells, cells from a cell line, cancer cells, infected cells, transfected and/or transformed cells, reporter cells, and the like. A mammalian cell can be, for example, from a human, a mouse, a rat, a horse, a goat, a sheep, a cow, a primate, or the like.

A colony of biological cells is “clonal” if all of the living cells in the colony that are capable of reproducing are daughter cells derived from a single parent cell. In certain embodiments, all the daughter cells in a clonal colony are derived from the single parent cell by no more than 10 divisions. In other embodiments, all the daughter cells in a clonal colony are derived from the single parent cell by no more than 14 divisions. In other embodiments, all the daughter cells in a clonal colony are derived from the single parent cell by no more than 17 divisions. In other embodiments, all the daughter cells in a clonal colony are derived from the single parent cell by no more than 20 divisions. The term “clonal cells” refers to cells of the same clonal colony.

As used herein, a “colony” of biological cells refers to 2 or more cells (e.g., about 2 to about 20, about 4 to about 40, about 6 to about 60, about 8 to about 80, about 10 to about 100, about 20 to about 200, about 40 to about 400, about 60 to about 600, about 80 to about 800, about 100 to about 1000, or greater than 1000 cells).

As used herein, the term “maintaining (a) cell(s)” refers to providing an environment comprising both fluidic and gaseous components and, optionally a surface, that provides the conditions necessary to keep the cells viable and/or expanding.

As used herein, the term “expanding” when referring to cells, refers to increasing in cell number.

As referred to herein, “gas permeable” means that the material or structure is permeable to at least one of oxygen, carbon dioxide, or nitrogen. In some embodiments, the gas permeable material or structure is permeable to more than one of oxygen, carbon dioxide and nitrogen and may further be permeable to all three of these gases.

A “component” of a fluidic medium is any chemical or biochemical molecule present in the medium, including solvent molecules, ions, small molecules, antibiotics, nucleotides and nucleosides, nucleic acids, amino acids, peptides, proteins, sugars, carbohydrates, lipids, fatty acids, cholesterol, metabolites, or the like.

As used herein in reference to a fluidic medium, “diffuse” and “diffusion” refer to thermodynamic movement of a component of the fluidic medium down a concentration gradient.

The phrase “flow of a medium” means bulk movement of a fluidic medium primarily due to any mechanism other than diffusion and may encompass perfusion. For example, flow of a medium can involve movement of the fluidic medium from one point to another point due to a pressure differential between the points. Such flow can include a continuous, pulsed, periodic, random, intermittent, or reciprocating flow of the liquid, or any combination thereof. When one fluidic medium flows into another fluidic medium, turbulence and mixing of the media can result. Flowing can comprise pulling solution through and out of the microfluidic channel (e.g., aspirating) or pushing fluid into and through a microfluidic channel (e.g., perfusing).

The phrase “substantially no flow” refers to a rate of flow of a fluidic medium that, when averaged over time, is less than the rate of diffusion of components of a material (e.g., an analyte of interest) into or within the fluidic medium. The rate of diffusion of components of such a material can depend on, for example, temperature, the size of the components, and the strength of interactions between the components and the fluidic medium.

As used herein in reference to different regions within a microfluidic device, the phrase “fluidically connected” means that, when the different regions are substantially filled with fluid, such as fluidic media, the fluid in each of the regions is connected so as to form a single body of fluid. This does not mean that the fluids (or fluidic media) in the different regions are necessarily identical in composition. Rather, the fluids in different fluidically connected regions of a microfluidic device can have different compositions (e.g., different concentrations of solutes, such as proteins, carbohydrates, ions, or other molecules) which are in flux as solutes move down their respective concentration gradients and/or fluids flow through the device.

As used herein, a “flow path” refers to one or more fluidically connected circuit elements (e.g., channel(s), region(s), chamber(s) and the like) that define, and are subject to, the trajectory of a flow of medium. A flow path is thus an example of a swept region of a microfluidic device. Other circuit elements (e.g., unswept regions) may be fluidically connected with the circuit elements that comprise the flow path without being subject to the flow of medium in the flow path.

As used herein, “isolating a micro-object” confines a micro-object to a defined area within the microfluidic device.

As used herein, “pen” or “penning” refers to disposing micro-objects within a chamber (e.g., a sequestration pen) within the microfluidic device. Forces used to pen a micro-object may be any suitable force as described herein such as dielectrophoresis (DEP), e.g., an optically actuated dielectrophoretic force (OEP); gravity; magnetic forces; or tilting. In some embodiments, penning a plurality of micro-objects may reposition substantially all the micro-objects. In some other embodiments, a selected number of the plurality of micro-objects may be penned, and the remainder of the plurality may not be penned. In some embodiments, when selected micro-objects are penned, a DEP force, e.g., an optically actuated DEP force or a magnetic force may be used to reposition the selected micro-objects. Typically, micro-objects may be introduced to a flow region, e.g., a microfluidic channel, of the microfluidic device and introduced into a chamber by penning.

As used herein, “unpen” or “unpenning” refers to repositioning micro-objects from within a chamber, e.g., a sequestration pen, to a new location within a flow region, e.g., a microfluidic channel, of the microfluidic device. Forces used to unpen a micro-object may be any suitable force as described herein such as but not limited to, dielectrophoresis, e.g., an optically actuated dielectrophoretic force; gravity; optically driven bubble(s); displacing fluid flow; magnetic forces; or tilting. In some embodiments, unpenning a plurality of micro-objects may reposition substantially all the micro-objects. In some other embodiments, a selected number of the plurality of micro-objects may be unpenned, and the remainder of the plurality may not be unpenned. In some embodiments, when selected micro-objects are unpenned, a DEP force, e.g., an optically actuated DEP force or a magnetic force may be used to reposition the selected micro-objects.

As used herein, “displace,” “displacing,” or “displacement” refers to repositioning micro-objects from a first location to a second location within a microfluidic device. The first location and the second location are independently within a chamber (e.g., a sequestration pen) or a flow region (e.g., a microfluidic channel) of the microfluidic device. Forces used to unpen a micro-object may be any suitable force as described herein such as but not limited to, dielectrophoresis, e.g., an optically actuated dielectrophoretic force; gravity; optically driven bubble(s); displacing fluid flow; magnetic forces; or tilting. In some embodiments, displacing a plurality of micro-objects may reposition substantially all the micro-objects. In some other embodiments, a selected number of the plurality of micro-objects may be displaced, and the remainder of the plurality may not be displaced. In some embodiments, when selected micro-objects are displaced, a DEP force, e.g., an optically actuated DEP force or a magnetic force may be used to reposition the selected micro-objects.

As used herein, “export” or “exporting” refers to repositioning micro-objects from a location within a flow region, e.g., a microfluidic channel, of a microfluidic device to a location outside of the microfluidic device, such as a 96 well plate or other receiving vessel. The orientation of the chamber(s) having an opening to the microfluidic channel permits easy export of micro-objects that have been positioned or repositioned (e.g., unpenned from a chamber) to be disposed within the microfluidic channel. Micro-objects within the microfluidic channel may be exported without requiring disassembly (e.g., removal of the cover of the device) or insertion of a tool into the chamber(s) or microfluidic channel to remove micro-objects for further processing.

A microfluidic (or nanofluidic) device can comprise “swept” regions and “unswept” regions. As used herein, a “swept” region is comprised of one or more fluidically interconnected circuit elements of a microfluidic circuit, each of which experiences a flow of medium when fluid is flowing through the microfluidic circuit. The circuit elements of a swept region can include, for example, regions, channels, and all or parts of chambers. As used herein, an “unswept” region is comprised of one or more fluidically interconnected circuit element of a microfluidic circuit, each of which experiences substantially no flux of fluid when fluid is flowing through the microfluidic circuit. An unswept region can be fluidically connected to a swept region, provided the fluidic connections are structured to enable diffusion but substantially no flow of media between the swept region and the unswept region. The microfluidic device can thus be structured to substantially isolate an unswept region from a flow of medium in a swept region, while enabling substantially only diffusive fluidic communication between the swept region and the unswept region. For example, a flow channel of a micro-fluidic device is an example of a swept region while an isolation region (described in further detail below) of a microfluidic device is an example of an unswept region.

As used herein, a “non-sweeping” rate of fluidic medium flow means a rate of flow sufficient to permit components of a second fluidic medium in an isolation region of the sequestration pen to diffuse into the first fluidic medium in the flow region and/or components of the first fluidic medium to diffuse into the second fluidic medium in the isolation region; and further wherein the first medium does not substantially flow into the isolation region.

### Methods for Facilitated Cell Displacement

Micro-objects may be moved in their local environment, such as within a microfluidic device by a number of forces, including, but not limited to gravity, fluidic flow induced by a mechanical pump, electrowetting and/or dielectrophoresis (DEP). In some circumstances, micro-objects may have attached to a surface of the microfluidic device, for example, a cell may be attached to a surface via interaction with surface fouling proteins produced by itself or other cells present within the microfluidic device. Such attachment may decrease the portability of the micro-objects. Optical illumination of discrete selected regions on or within a microfluidic device can heat a portion of a fluidic medium within the microfluidic circuit of the microfluidic device to provide a variety of displacement forces within the microfluidic device thereby facilitating the displacement of the micro-objects. However, optical illumination and the created displacement force can be harmful to the micro-objects if applied directly on them.

Methods and compositions for microfluidic workflows and processes are provided herein for which improvements to micro-object displacement process are obtained. Provided herein are piston structures having in situ-generated hydrogels, the use of which can result in increased cell displacement speeds and rates and reducing (or minimizing) of contact between micro-objects of interest and displacement forces. In some embodiments, the piston defines a target region within the chamber configured for displacement force generation. In some embodiments, the piston divides a chamber, e.g., a sequestration pen, into two areas, one more proximal to the pen opening which opens to a flow region and a second area distal to the pen opening and to the micro-objects of interest. The distal area, in some embodiments, is used exclusively as a target region (displacement force generation region), and the proximal area can be used for accommodating or culturing cells prior to unpenning.

In some embodiments, prior to, during, and/or after the formation of the in situ-generated barriers, an assay can be performed within an area of interest suitable for the selected assay. In some embodiments, an assay is performed to facilitate identification and selection of pens comprising cells of interest. An assay may be of any type, for example but not limited to, an assay for determining the bioproductivity of the cell, viability of the cell, expansion rate of the cell, and the like. The present disclosure is not limited by the presence or absence of performance of an assay, type of assay performed, or selection of cells of interest as a result of said assay. An assay may be as described in US Publication No. US2020/0408751 filed on Apr. 15, 2020, and U.S. Pat. No. 11,203,018 filed on Oct. 15, 2018 and issued on Dec. 21, 2021, each of which are incorporated herein by reference for the purposes described herein. In some embodiments, a hydrogel barrier (the in situ-generated piston or the in situ-generated guide element described herein) may have a porosity allowing selective permeability permitting one, some, or all of the one or more reagents associated with an assay and/or with cell culture to pass through the piston to the distal area of the chamber and vice versa.

Penning cells. In some embodiments, disposing a cell into a chamber of a microfluidic device comprises: obtaining a microfluidic device comprising a microfluidic circuit comprising a flow region and a chamber fluidically connected to the flow region; introducing a fluidic medium comprising the cell into the flow region; and disposing the cell into the chamber. In some embodiments, the cell is disposed into a chamber of a microfluidic device by gravity or by OEP as described herein. In some embodiments, positive OEP or negative OEP can be selected depending on the surface charge of the cell to be moved.

In some embodiments, when the cell to be deposited within the microfluidic device (e.g., the cell to be penned) is small (e.g., nonmammalian cells, which tend to be smaller than mammalian cells), for example, smaller than 10 microns in diameter, the OEP is performed at a higher voltage. In some embodiments, the voltage is higher than 5, 6, 7, 8, 9, 10, 11, 12, 13, 14, or 15V. In some embodiments, the diameter of the cell is about 0.5, 1, 1.5, 2, 2.5, 3, 3.5, 4, 4.5, 5, 5.5, 6, 6.5, 7, 7.5, 8, 8.5, 9, 9.5, 10 microns.

The process of penning cells may be automated using image recognition software as described in U.S. Patent Publication No. 11,170,200 (Kim et al.), filed on May 31, 2019 and granted Nov. 9, 2021, and U.S. Application Publication No. US2021/0209752 (Tenney et al.), filed on Nov. 24, 2020, each of which disclosures is herein incorporated in its entirety by reference. In various embodiments, the cells are moved into separate chambers, e.g., NanoPen® chambers, to be cultured as individual colonies. By penning a single cell to an individual chamber, expansion to a population of cells provides a clonal population. The ability to observe, test, and export selectively a specific clonal population of cells that exhibit desired characteristics, provided by the methods described herein, is an important improvement over the macroscale techniques currently used for developing engineered cell lines that can produce a desired bioproduct. To more effectively harness the potential for assay processes carried out within each of many individual chambers within the microfluidic device, barriers can be introduced into the chamber for a number of purposes that may improve the assay by enhancing the accuracy, sensitivity, or reproducibility, amongst other properties, of the assay. For example, a barrier may be introduced, e.g., generated in situ, in order to sequester cells away from or within an area, such as but not limited to, sequestering cells outside of a target area, sequestering cells within or outside of a pen, sequestering cells within a culture area, and/or sequestering cells within or outside of an assay observation area (e.g., area of interest, e.g., so a rapidly secreting cell does not artificially enhance the detected signal for the entire chamber by being present within an area of interest as a point source). Barriers may also be introduced to prevent a secreted analyte that is bound to a reporter molecule (RMSA complex) from diffusing away from an area of interest. Barriers also can prevent molecules having a size (molecular weight) too large to pass through the barrier to reach an area of interest of an assay which might interfere with the assay mechanism.

In situ-generated barrier. As disclosed herein, an in situ-generated barrier is formed within a chamber of a microfluidic device. Generally, one of the functions of the in situ-generated barrier in the methods of the present disclosure is to contain a cell within a certain chamber, region, or area of the microfluidic device. The term “in situ-generated barrier” refers to a barrier that is formed in a selected area while the microfluidic device is in operation. The barrier is generally not formed while manufacturing the microfluidic device or does not exist before the microfluidic device is used for experiments or research. The term “barrier” refers to a physical structure that is formed and fixed, at least for a certain period of time, in a selected area and is capable of impeding or blocking a micro-object (such as but not limited to, a cell) from crossing through the barrier. As a result, the barrier formed in situ within the chamber can separate the inner space thereof into two areas on each side of the barrier. In some embodiments, the barrier defines a culture area and an excluded culture area within the chamber (e.g., a target region).

In some embodiments, an in situ-generated barrier is a piston. In some embodiments, a piston is formed in situ within a chamber comprised in a microfluidic device. In some embodiments, following piston formation, two areas on either side of the piston are defined, and are distinct regions in the chamber. A first region is configured to comprise a plurality of micro-objects as an enclosed culture area (such as but not limited to cells), and in some embodiment, the first region is proximal to the opening of the chamber to the flow. A second region is configured to be substantially absent of micro-objects (such as but not limited to cells) and configured to receive a displacement force generation as a target region. In some embodiments, the second region is distal to the opening of the chamber.

In some embodiments, an in situ-generated barrier is a guide element. In some embodiments, a guide element is formed at a region of a chamber proximal to a microfluidic channel (i.e., proximal to the opening of the chamber) to impede or block micro-objects (such as but not limited to cells) from readily crossing the guide element without application of a sufficient force, such as a displacement force. In some embodiments, such a barrier can be utilized to create an enclosed culture area. For example, in some embodiments, an in situ-generated guide element can be formed within a connection region between a chamber and a microfluidic channel. In some embodiments, such a guide element in the connection region can be configured to allow a micro-object to be unpenned when the micro-object is subject to a sufficient displacement force, and impede movement of the unpenned micro object from re-entering into the chamber. In some embodiments, an in situ-generated barrier can be of any suitable shape, such as but not limited to, a circle, an oval, a square, a teardrop, and the like.

As used herein, “culture area” refers to an area pre-determined for maintaining or culturing a cell, for any desired period of time, but the methods of the present disclosure are not limited to maintain or culture the cell in the culture area.

In some embodiments, the impediment or block produced by introduction of the barrier is size-dependent. A particle can be impeded, blocked, or allowed to cross through the barrier depending upon its size. In some embodiments, the in situ-generated barrier has a porosity that substantially prevents a micro-object (e.g., a cell) from crossing through the in situ-generated barrier. In some embodiments, the in situ-generated barrier can comprise a gap having a width or diameter that allows a micro-object (e.g., a cell) to pass through the barrier. Nevertheless, the movement of micro-objects (e.g., cells) through the barrier via the gap can still be impeded.

Hydrogel in situ-generated barrier. In certain embodiments, the in situ-generated barrier is a hydrogel. In certain embodiments, the in situ-generated barrier comprises a solidified polymer network. In some embodiments, the solidified polymer network comprises a synthetic polymer, a modified synthetic polymer, or a biological polymer. In certain embodiments, the solidified polymer network comprises at least one of a polyethylene glycol, modified polyethylene glycol, polyglycolic acid (PGA), modified polyglycolic acid, polyacrylamide (PAM), modified polyacrylamide, poly-N-isopropylacrylamide (PNIPAm), modified poly-N-isopropylacrylamide, polyvinyl alcohol (PVA), modified polyvinyl alcohol, polyacrylic acid (PAA), modified polyacrylic acid, fibronectin, modified fibronectin, collagen, modified collagen, laminin, modified laminin, polysaccharide, modified polysaccharide, or a co-polymer in any combination. In some embodiments, the solidified polymer network does not include a silicone polymer.

Physical and chemical characteristics determining suitability of a polymer for use in the solidified polymer network may include molecular weight, hydrophobicity, solubility, rate of diffusion, viscosity (e.g., of the medium), excitation and/or emission range (e.g., of fluorescent reagents immobilized therein), known background fluorescence, characteristics influencing polymerization, and pore size of a solidified polymer network. The solidified polymer network is formed upon polymerization or thermal gelling of a flowable polymer solution containing at least one of a polyethylene glycol, modified polyethylene glycol, polyglycolic acid (PGA), modified polyglycolic acid, polyacrylamide (PAM), modified polyacrylamide, poly-N-isopropylacrylamide (PNIPAm), modified poly-N-isopropylacrylamide, polyvinyl alcohol (PVA), modified polyvinyl alcohol, polyacrylic acid (PAA), modified polyacrylic acid, fibronectin, modified fibronectin, collagen, modified collagen, laminin, modified laminin, polysaccharide, modified polysaccharide, or a co-polymer in any combination. Various co-polymer classes may be used, including but not limited to any of the above listed polymers, or biological polymers such as fibronectin, collagen or laminin. Polysaccharides such as dextran or modified collagens may be used. The flowable polymer may be referred alternatively here as a pre-polymer, in the sense that the flowable polymer is crosslinked in-situ. Biological polymers having photoactivatable functionalities for polymerization may also be used.

In some instances, a polymer may include a cleavage motif. A cleavage motif may include a peptide sequence inserted into the polymer that is a substrate for one or more proteases, including but not limited to a matrix metalloproteinase, a collagenase, or a serine proteinase such as Proteinase K. Another category of cleavage motif may include a photocleavable motif such as a nitrobenzyl photocleavable linker which may be inserted into selected locations of the prepolymer. In some embodiments, a nitrobenzyl photocleavable linker may include a 1-methinyl, 2-nitrobenzyl moiety configured to be photocleavable. In other embodiments, the photocleavable linker may include a benzoin moiety, a 1, 3 nitrophenolyl moiety, a coumarin-4-ylmethyl moiety or a 1-hydroxy 2-cinnamoyl moiety. A cleavage motif may be utilized to remove the solidified polymer network of an isolation structure. In other embodiments, the polymer may include cell recognition motifs including but not limited to a RGD peptide motif, which is recognized by integrins.

One type of polymer, amongst the many polymers that may be used, is polyethylene glycol diacrylate (PEGDA) or polyethylene glycol acrylamide (diacrylamide, multi-armed acrylamide or substituted versions as described herein).

Photoactivated polymerization may be accomplished using a free radical initiator Igracure® 2959 (BASF), a highly efficient, non-yellowing radical, alpha hydroxy ketone photoinitiator, is typically used for initiation at wavelengths in the UV region (e.g., 365 nm), but other initiators may be used. An example of another useful photoinitiator class for polymerization reactions is the group of lithium acyl phosphinate salts, of which lithium phenyl 2, 4, 6,-trimethylbenzolylphosphinate has particular utility due to its more efficient absorption at longer wavelengths (e.g., 405 nm) than that of the alpha hydroxy ketone class. Another initiator that may be used are water soluble azo initiators, such as 2, 2-Azobis [2-methyl-N-(2-hydroxyethyl) propionamide]. The initiator may be present within the flowable polymer solution at a concentration of about 5 millimolar, about 8 millimolar, about 10 millimolar, about 12 millimolar, about 15 millimolar, about 18 millimolar, about 20 millimolar, about 22 millimolar, about 25 millimolar, about 28 millimolar, about 30 millimolar, about 35 millimolar, or about 40 millimolar.

Crosslinking may be performed by photopatterning of linear or branched PEG polymers, free radical polymerization of PEG acrylates or PEG acrylamides, and specifically tailored chemical reactions such as Michael addition, condensation, Click chemistry, native chemical ligation and/or enzymatic reactions. In particular, photopatterning of crosslinking may be used to gain precise control of extent of the physical extent of the hydrogel barrier as well as the degree of crosslinking, as described in the following section and in the Examples.

Inhibitors may be included within the flowable polymer solution to ensure precise control of photopatterning and to prevent extraneous or undesired polymerization. One useful inhibitor is hydroquinone monomethyl ether, MEHQ, but other suitable inhibitors may be used. The inhibitor may be present in the flowable polymer solution at a concentration of about 1 millimolar, about 2 millimolar, about 5 millimolar, about 10 millimolar, about 15 millimolar, about 20 millimolar, about 25 millimolar, about 30 millimolar, about 35 millimolar, about 40 millimolar or more, as needed to provide the photopatterning control desired.

Tuneable permeability. One aspect of performing assays using a hydrogel in a situ-generated barrier is to determine what species is desired to gain access to the area of interest. Selection of the chemical nature of the polymer (for example, molecular weight range, number of cross linkable moieties per polymer unit (linear, 2 arm, 4 arm, 8 arm, star or comb polymer), mixtures of polymers), the amount of initiator, and mode of polymerization are variables that may be modified to tune the hydrogel barrier formed. Generally, the initiator is a photoinitiator. Photopatterning provides precise control of the geometry of the polymerization as well as the extent of polymerization, and changes in exposure time and power of the illumination also can provide more control to arrive at a desired type of porosity and degree of robustness of the polymerized feature.

In many variations, polymer selection may depend upon the biocompatibility of the polymer species, and may be related to the specific application to which a hydrogel in situ-generated barrier may be used.

In some variations, the hydrogel may be a polyethylene glycol polymer or a modified polyethylene glycol polymer.

A wide range of molecular weights of the flowable polymer may be suitable, depending upon the structure of the polymer. In some embodiments, the flowable polymer may have a molecular weight of about 500 Da to about 20 kDA, or about 500 Da, about 1 kDa, about 3 kDa, about 5 kDa, about 10 kDa, about 12 kDa, about 15 kDa, about 20 kDa or any value therebetween. A useful star type polymer may have Mw (weight average molecular weight) in a range from about 500 Da to about 20 kDa (e.g., four arm polymer), or up to about 5 kDa for each arm, or any value therebetween. In some embodiments, a polymer having a higher molecular weight range, may be used at lower concentrations in the flowable polymer, and still provide an in situ-generated piston or other types of in situ-generated barriers that may be used in the methods described herein.

Cell exportation and geometry of the in situ-generated barriers. The in situ-generated barriers, such as pistons, of the methods of the present disclosure provides advantages of separating a cell from a displacement force generation region within the chamber in which the cell is disposed or has been cultured. In some embodiments, the methods of the present disclosure comprise unpenning a cell from the chamber. In some embodiments, the cell is further exported out of the microfluidic device. In some embodiments, various mechanisms may be used to remove, reduce, or bypass an in situ-generated hydrogel barrier, such as a piston and/or connection region barrier, such that a cell can be unpenned. In some embodiments, displacing the cell comprises directing a laser illumination upon a selected area of the chamber (e.g., a displacement force generation region), to create a bubble pushing towards the piston, the piston can then be deformed, displaced, flipped, slipped, and/or otherwise pushed toward the cell by the displacement force associated with the bubble, and subsequently the piston, expanding fluid, and/or bubble can push the cell toward the opening of the chamber. In other embodiments, unpenning the cell comprises directing laser illumination upon a selected area of the chamber (e.g., a displacement force generation region) to create a bubble pushing towards the piston, the piston can then be deformed, displaced, flipped, slipped, and/or otherwise pushed toward the cell by the displacement force associated with the bubble, and subsequently the piston, expanding fluid, and/or bubble push the cell towards the opening of the chamber, and dielectrophoretic forces may then be used to unpen the cell. In some embodiments, the displacement of the piston is proportional to the power and/or duration of the illumination.

Without intending to be limited by theory, the laser illumination is set to project on a selected site on a surface of the chamber, such as a site within a displacement force generation region, to generate heating in the fluidic medium surrounding the selected site on a surface of the chamber, which may nucleate and propagate bubbles on the displacement force generation region side of a piston. In some embodiments, a selected site on a surface of the chamber is within a displacement force generation region defined by a piston, e.g., a region distal to the chamber (e.g., pen) opening. In some embodiments, the bubble may be grown by continued illumination to create a shear flow of fluid directed towards nearby substances, such as towards the piston. The force and/or the flow can push the in situ-generated piston, fluidic medium, and micro-objects in a cell culture area away, typically in a direction towards the opening of the chamber. As a result, once the in situ-generated piston no longer holds its original position the micro-objects can be moved out of the chamber. In some embodiments, the micro-objects (e.g., cells) are moved by the piston, expanding fluid (e.g., through heat), the bubble, and/or forces generated by the bubble, to a location close to the opening of the chamber to the microfluidic channel, and/or into the microfluidic channel. In some embodiments, the cell is moved by the bubble to a location close to an opening of the chamber to the microfluidic channel, and an OEP force is applied to further move the cell into the microfluidic channel where later the cell is flushed out of the microfluidic device by a flow introduced therein. In some other embodiments, once the laser illumination pulse has been stopped, the induced bubble collapses, creating a cavitating force, resultingly drawing fluid back towards the distal end of the chamber. In some embodiments, an in situ-generated guide element can be created and utilized to impede backflow of micro-objects into the chamber following bubble collapse and/or fluid cooling.

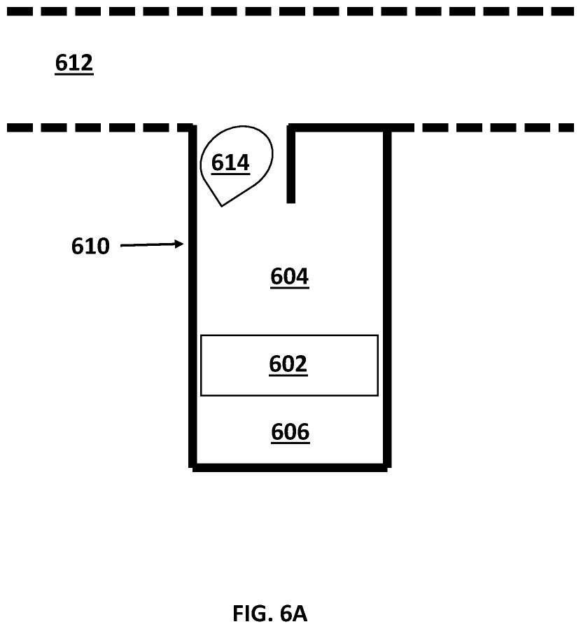

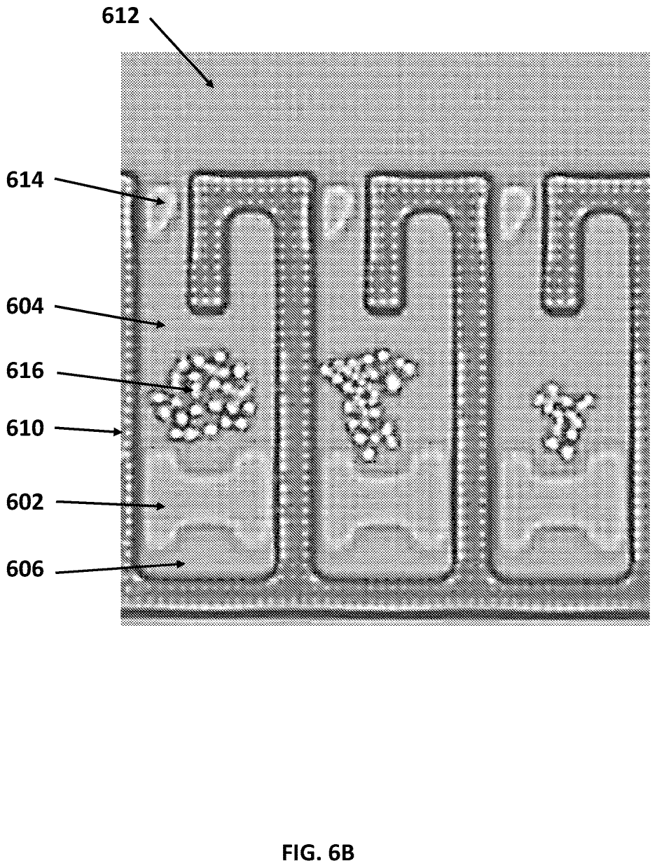

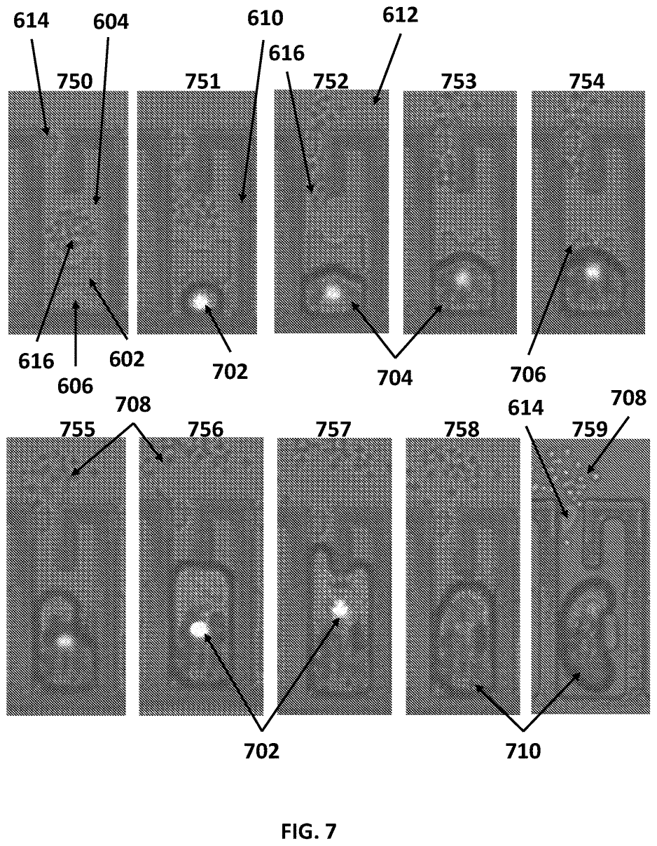

In some embodiments, the site of illumination may be selected to be any discrete selected site on the surface of the chamber within a target region. In some embodiments, the discrete selected region of illumination may be a location within a chamber (e.g., a sequestration pen) of a microfluidic device. In various embodiments, the discrete selected region of illumination is located within an isolation region of a sequestration pen, which may be configured like any sequestration pen described herein. In some embodiments, the laser illumination is projected at a cell-free area of the chamber, such as an area defined by the piston as a target region. In some embodiments, the laser illumination is projected at the target region area near the distal end of the chamber. In certain embodiments, an OEP force is applied first to move the cell cultured in the chamber away from the distal end thereof to create a cell-free area, an in situ-generated piston is then formed, and then a laser illumination can be applied at the substantially cell-free or cell-free target region to create a bubble. One method of exporting cells using laser illumination in the presence of a hydrogel piston is further discussed below and is shown in FIGS. 6A-B, and FIG. 7.

The geometry of the in situ-generated piston can be selected to facilitate the cell unpenning. Without intending to be bound by any theory, in some embodiments, the in situ-generated piston is designed to have a structurally vulnerable portion so that, upon application of a threshold pressure (for instance, a force generated by the bubble created by laser illumination), the in situ-generated barrier can be deformed, displaced, flipped, slipped, or otherwise pushed from its original position, or changed of position, resulting in transfer of a force to the cells, thus moving them. In other words, in some embodiments, the in situ-generated piston will facilitate movement of the cell into the microfluidic channel after the laser illumination.

Accordingly, various exemplary shapes of in situ-generated piston can be formed within the chamber. These include, but are not limited to, a bar (extended from both sides of the wall, or with one or more gaps between the bar and the walls), a rectangular half or three-quarters bar, a simple or convex polygon, a dart polygon, a complex polygon, an irregular polygon, a rectangular polygon comprising an additional thickness at the ends, a side bars (two discrete side rectangular bars separated by a gap and extended from both sides of the wall, a single triangular bar, a V-shaped bar, and a V-cap (a rectangular bar having a v shaped depression on the side of the piston facing the opening to the channel. In some embodiments, an in situ-generated piston can have one or more gaps,

Further the size of the components of the hydrogel pistons may vary considerably. In some embodiments, nonuniform pistons may have a variety of dimensions that are nonuniform. Non-uniformity may be located in the width (pen wall to pen wall dimension), in the “thickness” dimension (from distal-to-pen opening to proximal-to-pen opening dimension), and/or the “height” dimension (from inner surface of the substrate to the inner surface of the cover of the microfluidic device, e.g., z-dimension).

In some embodiments, an in situ-generated guide element can be of various shapes, but includes a gap suitably sized to allow unpenning of micro-objects that are subject to an unpenning force, but sized suitably to impede entry of micro-objects into a pen following removal of an unpenning force. In some embodiments, the shape of an in situ-generated guide element can be, but is not limited to, a bar (extended from both sides of the walls of a connection region, but with one or more gaps between the bar and the walls), a rectangular half or three-quarters bar, a simple or convex polygon, a dart polygon, a concave polygon, a complex polygon, a trapezoid, an irregular polygon, a triangle, a wedge, and/or a tear drop.

As shown in the graphical example of FIG. 6A, a sequestration pen 610 of a microfluidic device prepared for unpenning of a micro-objects comprises at least one in situ-generated barrier, a piston 602. The generation of the piston defines two regions of the pen 610, a culture region 604 more proximal to the opening of the pen 610 to the microfluidic channel 612 and configured to comprise a plurality of micro-objects (such as but not limited to cells), and a target region 606 more distal to the opening of the pen 610 to the microfluidic channel 612 and configured to be substantially absent or absent of micro-objects. Also shown is an optional additional in situ-generated barrier, an in situ-generated guide element 614 established towards the proximal end of pen 610 with an opening to microfluidic channel 612. The generation of the in situ-generated guide element 614 can facilitate impedance of micro-objects re-entering a pen from a microfluidic channel 612 following unpenning. The piston 602 and the in situ-generated guide element 614 can be implemented alone or in combination. That is, piston 602 can be used with or without in situ-generated guide element 614. Likewise, in-situ generated guide element 614 can be used with or without piston 602.

As shown in the photographic example of FIG. 6B, a sequestration pen 610 of a microfluidic device prepared for unpenning of micro-objects 616 comprises at least one in situ-generated barrier, a piston 602. As depicted in FIG. 6B, piston 602 is indented in the middle on both sides such that the side facing the distal end of sequestration pen 610 and the side facing the proximal end of sequestration pen 610 provide a structurally vulnerable portion. That is, the thickness of piston 602 varies when observed from above, with the thickness larger along the walls of the sequestration pen 610 that extend from the proximal opening to the distal end, and thinner in the middle between the two thicker portions. This geometry may aid in displacing micro-objects 616 towards or within microfluidic channel 612, as described herein. However, in some implementations, the indentation may only be on the side of piston 602 that is facing or adjacent to target region 606, and the other side of piston 602 may have a different shape (e.g., no indentation or substantially planar, with the planar side being perpendicular to the axis defined by the proximal opening and the distal end of the pen or, alternatively, with the planar side being angled toward the proximal opening of the pen). The indentation on the side facing target region 606 may be useful in facilitating improved bubble formation and direction of the corresponding displacement force described herein.

Additionally, the generation of the piston defines two regions of the pen 610, a culture region 604 more proximal to the opening of the pen 610 to the microfluidic channel 612 and configured to comprise a plurality of micro-objects 616 (such as but not limited to cells, as shown in the picture), and a target region 606 more distal to the opening of the pen 610 to the microfluidic channel 612 and configured to be substantially absent or absent of micro-objects 616. Also shown is an optional additional in situ-generated barrier, an in situ-generated guide element 614. In situ-generated guide element 614 is “teardrop” shaped with a thicker portion closer to the opening of sequestration pen 610 (or closer to the microfluidic channel) and tapers to a thinner end as it is closer to the distal end of sequestration pen 610 (or farther away from the microfluidic channel). This geometry allows for a funnel-like effect aiding in displacement of micro-objects 616 into the microfluidic channel. Moreover, the generation of the in situ-generated guide element 614 serves as a connection region barrier that can facilitate impedance of micro-objects 616 re-entering a sequestration pen from a microfluidic channel 612 following unpenning or displacement.

As shown in the series of photographic representations of a time lapsed unpenning example of FIG. 7, a sequestration pen 610 of a microfluidic device prepared for unpenning of a micro-object 616 comprises at least one in situ-generated barrier, a piston 602. The time lapse photographs 750, 751, 752, 753, 754, 755, 756, 757, 758, and 759 were taken over a period of 10 seconds, and exemplify unpenning of micro-objects 616 (such as but not limited to cells, as shown here) from a pen 610 through creation of a laser 702 initiated displacement force generated in the displacement force generation region 606. As pictured in 750, an in situ-generated piston 602 separates the pen into two distinct regions, a culture region 604 comprising micro-objects 616, and a target region 606 absent of micro-objects.

As pictured in 751, 752, and 753, a displacement force is generated in the target region, in this example through laser illumination 702 and bubble 704 creation, which results in fluid heating and/or expansion of the bubble pushing towards the piston 602, facilitating unpenning of the micro-objects 616 into the microfluidic channel 612. As pictured in 754, as the bubble 704 expands, the piston 602 is pushed and deformed, displaced, and/or otherwise moved 706 (deformed, displaced, and/or otherwise moved piston 602) in response to the expanding bubble 704.

Operational parameters related to how the laser operates can also be adjusted while laser illumination 702 is applied to further facilitate movement of micro-objects 616 towards the microfluidic channel. For example, as pictured in 755, 756, and 757 in FIG. 7, the position of the laser illumination 702 can be moved closer to the opening of the pen 610 from a starting point within target region 606 over a time period to further expand the bubble throughout the sequestration pen 610 and culture region 604.

In other implementations, the intensity of the laser illumination 702 (e.g., the power per unit area delivered by the laser illumination 702 incident upon the microfluidic device) can also be adjusted. As another example, the speed and/or acceleration of moving laser illumination 702 from the distal end of the sequestration pen 610 towards the proximal end closer to the opening to microfluidic channel can be adjusted. Moreover, any of the operational parameters described can be adjusted alone or in combination with any of the others. For example, any combination of adjusting position, intensity, speed, or acceleration of the laser illumination can be adjusted together.

As pictured in 758 and 759, following cessation of laser illumination 702, the bubble 710 begins to contract, drawing unpenned micro-objects 708 back towards the sequestration pen 610 opening, optional additional in situ-generated barriers such as in situ-generated guide elements 614 can impede movement of unpenned micro-objects 708 into sequestration pens 610. Therefore, the micro-objects 616 which were within the sequestration pen are moved towards the opening to the microfluidic channel and pass the in situ-generated guide elements 614 over time. When laser illumination 702 ceases, some of the fluidic medium might rush back into the sequestration pen and pull some of the micro-objects 616 back towards the opening of the sequestration pen. In some cases, some micro-objects 616 might move from the microfluidic channel and back into the sequestration pen. However, because the in situ-generated guide element 614 is formed at the opening of the sequestration pen to the microfluidic channel, most of the micro-objects 616 remain within the microfluidic channel and do not enter back into the sequestration pen. The flow of fluidic media within the microfluidic channel can then be resumed, sweeping the micro-objects 616 away for collection, or export for further analysis or usage. Thus, in situ generated guide elements 614 can prevent or reduce the micro-objects 616 that might move back into the sequestration pen, thereby providing more micro-objects 616 for further analysis.

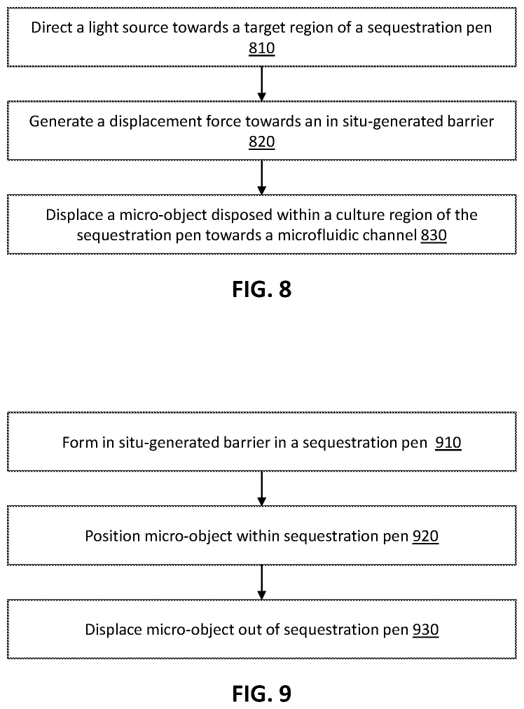

FIG. 8 is a block diagram for displacing a micro-object out of a sequestration pen of a microfluidic device. In FIG. 8, a light source is directed towards a target region of a sequestration pen (810). For example, in FIG. 6A, a light source (e.g., a laser) illuminates a surface of sequestration pen 610 that is within target region 606. Target region 606 is at the distal end of the sequestration pen from the opening to the microfluidic channel, separated from culture region 604 (in which a micro-object is disposed) by piston 602.

Next, the illumination of the light source in the target region generates a displacement force towards the in situ-generated barrier (820). For example, in FIG. 6A, the illumination using the light source heats the fluidic medium within target region 606. This results in the formation of a bubble which expands and pushes, deforms, displaces, and/or moves piston 602 in response to the expanding bubble. The micro-object within the culture region is then displaced towards the microfluidic channel (830). For example, in FIG. 6A, the micro-object is pushed out of sequestration pen 610 and into microfluidic channel 612.

FIG. 9 is another block diagram for displacing a micro-object out of a sequestration pen of a microfluidic device. In FIG. 9, an in situ-generated barrier is formed in a sequestration pen (910). For example, as discussed in the disclosure, a flowable hydrogel polymer and photoinitiator may be introduced into the microfluidic device and into sequestration pens. A light source (e.g., a DMD-based light source) is used to photopattern the flowable hydrogel polymer and photoinitiator, resulting in solidified hydrogel barriers with shapes based on the photopatterning. One example is depicted in FIG. 6B, in which piston 602 is the resulting hydrogel barrier. Next, a micro-object can be positioned within the sequestration pen (920). For example, a micro-object 616 can be positioned within the sequestration pen in between piston 602 and the opening of the sequestration pen to the microfluidic channel. In FIG. 6B, this is culture region 604. Thus, piston 602 is positioned within sequestration pen 610 such that it separates a target region 606 on the side of piston 602 closer to or facing the distal end of sequestration pen 610 from a culture region 604 on the side of piston 602 closer to or facing the proximal end of sequestration pen 610 to the opening to the microfluidic channel 612. Next, the micro-object is displaced out of the sequestration pen (930). For example, in FIG. 6B, micro-objects 616 can be displaced as discussed with respect to FIG. 8 and the other parts of the disclosure.

In some embodiments, piston 602 in FIG. 6B is formed and, therefore, defines target region 606 and culture region 604 on opposite sides of piston 602. Subsequently, micro-object 616 is disposed within culture region 604. Next, the micro-objects 616 can be displaced from sequestration pen 610 using the techniques described herein regarding bubble formation.

In another embodiment, piston 602 in FIG. 6B is formed and, therefore, defines target region 606 and culture region 604 on opposites sides of piston 602. Subsequently, micro-object 616 is disposed within culture region 604. Next, in situ-generated guide element 614 is formed at the opening of sequestration pen 610 to microfluidic channel 612. Finally, micro-objects 616 can be displaced from sequestration pen 610 using the techniques described herein regarding bubble formation with the aid of in situ-generated guide elements 614.

Devices and Method for displacing and/or unpenning one or more micro-objects using optically driven convection and micro-object displacement.

The optical illumination, power of the laser, and other information regarding bubble dislodgement have been described, for example, in U.S. Pat. No. 10,829,728 (Kurz et al., issued on Nov. 10, 2020) and U.S. Publication No. 2022/0033758 (Sackmann et al., published on Feb. 3, 2022), the contents of which is incorporated herein by reference in its entirety.

Micro-objects such as biological cells or embryos, may be moved in their local environment, such as within a microfluidic device by a number of forces, including, but not limited to gravity, fluidic flow induced by a mechanical pump, electrowetting and/or dielectrophoresis (DEP). In order to more effectively move micro-objects from one location (e.g. a specific location where the micro-objects may have been cultured within a microfluidic device, e.g., a pen) to another location (e.g. another area of the same microfluidic device, e.g., a microfluidic channel), varying force vectors may be applied to achieve cell translocation. While dielectrophoresis (DEP), fluid displacement, and the like may be sufficient to move cells in the desired manner, forces applied at different scale (e.g., a more powerful force or a more localized force), in different ways (convective forces, shear flow forces, impacting forces such as cavitation or contact with a meniscus of a bubble, or any combination thereof) and/or on different timescales (e.g., from milliseconds to minutes in duration) may also be employed to assist in moving cells from a present location and/or to a selected location. In one non-limiting example, application of forces in addition to, and/or other than DEP may be useful to move biological cells that have been cultured within a microfluidic device for a period of time. In some embodiments, the cells may have attached to a surface of the microfluidic device, and DEP forces may be sufficient to move the cells from the attached position. In other embodiments, DEP forces or gravity may not be sufficient to move the cells from an attached position.

Optical illumination of discrete selected regions on or within a microfluidic device can heat a portion of a fluidic medium within the microfluidic circuit of the microfluidic device to provide a variety of displacement forces differing in scale, physical type and/or timescale which are capable of displacing micro-objects (including but not limited to biological cells) and/or mixing fluidic media (which may contain micro-objects including biological cells) within the microfluidic device. As described herein, in situ-generated barriers such as pistons can be generated to facilitate segregation of culture regions from force generation regions, facilitating displacement of micro-objects while retaining high levels of viability. The generation of such displacing forces may be applied more than once at the same discrete selected region or adjacent thereto (e.g., a selected site within a force generation region), such that repeated force can be applied to unpen micro-objects and/or mix media (which may include micro-objects), while being sufficiently non-destructive towards micro-objects. Translocating cells from one area, which in some embodiments may be a chamber, sequestration pen, or other microfluidic circuit element of a microfluidic device, to another area and/or location within the microfluidic device, may be accomplished by applying a pulse of optical illumination to selected discrete regions within the microfluidic device (e.g., a selected site within a force generation region). The force vector applied is a function of the energy, duration, and location of the pulse of optical illumination. In some embodiments, the pulse of optical illumination can be used to locally heat the surrounding cell media (i.e. fluidic medium), thereby increasing the local vapor pressure to create a vapor-fluid interface that produces a bubble. The effect upon the surrounding fluidic media and/or cell(s) of the heat-induced bubble generation may vary, depending on the duration and configuration of the microfluidic device and/or the thermal target. Some variety of effects may include:

Cavitation. A short pulse of light may be used to heat the thermal target to generate a short-lived bubble. The bubble creates a cavitating force acting to propel the piston that may dislodge cells disposed in the proximal region of the chamber.

Shear Flow. In other embodiments, the bubble may be grown by continued illumination to create a shear flow of fluid directed towards the piston and thereby dislodge and/or unpen the micro-object(s).

Meniscus Contact. Alternatively, the bubble(s) created by heating the fluidic medium at the site of the thermal target may be directed towards the piston. As the bubble moves, the meniscus of the bubble may propel the piston to displace cells disposed in the proximal region of the chamber.

In other embodiments, the bubble can be grown until it is thermodynamically favorable to stabilize and persist in the fluidic medium. The bubble may then displace the surrounding liquid phase and propel the piston to displace cells disposed in the proximal region of the chamber.

Optical Illumination. The optical illumination can be a coherent light source (e.g., a laser) or a non-coherent light source. The coherent light source may be a laser characterized by a wavelength in the visible light spectrum (e.g., a red wavelength, such as 662 nm), or may be a laser characterized by a wavelength in the infrared part of the spectrum (e.g., a near infrared wavelength, such as 785 nm), or may be a laser having any other suitable wavelength. The non-coherent light source may contain light having wavelengths in the visible range, and/or may include light having wavelengths in the ultraviolet (uv) or infrared range. The light source may provide structured or unstructured light. Temperature gradients introduced by illumination with a light source, may be modulated by increasing or decreasing the intensity of the light source. A structured light source may be modulated in a number of ways to control the properties of the structured light source (e.g. using a DMD to spatially modulate the light source, or using an aperture and objective to modulate the lights source.

Without being bound by theory, the incident optical illumination may be transmitted through a transparent, substantially transparent, and/or translucent cover or base of the enclosure microfluidic device. After being transmitted through the cover or base of the enclosure, the incident illumination can be transmitted to a thermal target, as described below, which is configured to convert the optical illumination to thermal energy.

Power. Non-coherent light may be projected in a range from about 1 milliwatts (mW) to about 1000 milliwatts (mW), but is not limited to this range. In some embodiments, the power of the non-coherent light, structured or non-structured may be in a range of about 1 milliwatt to about 500 milliwatts; about 1 milliwatt to about 100 milliwatts; about 1 milliwatt to about 50 milliwatts; about 1 milliwatt to about 20 milliwatts; about 10 milliwatts to about 500 milliwatts; about 10 milliwatts to about 200 milliwatts, about 10 milliwatts to about 100 milliwatts; about 50 milliwatts to about 800 milliwatts; about 50 milliwatts to about 500 milliwatts; about 50 milliwatts to about 200 milliwatts; about 75 milliwatts to about 700 milliwatts; about 75 milliwatts to about 400 milliwatts; about 75 milliwatts to about 175 milliwatts, or any value therebetween. Depending on the area to which the light is focused, and on the duration of illumination, the power of the non-coherent light may be less or more than any of the power levels described above.

Coherent light may be projected in a range from about 1 milliwatts to about 1000 milliwatts, but is not limited to this range. Depending on the area to which the light is focused, and on the duration of illumination, the power of the coherent light may be less or more than any of the power levels described above. In some embodiments, the power of the coherent light may be in a range of 1 milliwatt to about 500 milliwatts; about 1 milliwatt to about 100 milliwatts; about 1 milliwatt to about 50 milliwatts; about 1 milliwatt to about 20 milliwatts; about 10 milliwatts to about 500 milliwatts; about 10 milliwatts to about 200 milliwatts, about 10 milliwatts to about 100 milliwatts; about 50 milliwatts to about 800 milliwatts; about 50 milliwatts to about 500 milliwatts; about 50 milliwatts to about 200 milliwatts; about 75 milliwatts to about 700 milliwatts; about 75 milliwatts to about 400 milliwatts; about 75 milliwatts to about 175 milliwatts, or any value therebetween.

The power of the incident light may be chosen to be different based on the type of dislodging and/or unpenning force desired. For example, if a cyclized flow which may incorporate a Marangoni-effect flow is desired, the power of the incident light may be selected to be as low as 1 milliwatt and modulated variously as the cyclized flow is established and/or maintained. When dislodging micro-objects by use of a cavitating force, shear flow force, or bubble contact force the power may be selected to be in a higher range, for example from about 10 milliwatts to about 100 milliwatts. The power may also be adjusted based on the duration of the illumination desired as well.

Site of illumination. A site of illumination may be selected to be any discrete selected region of the microfluidic device, as may be useful. In most embodiments, at least one site of illumination is within a site for displacement force generation. In some embodiments, a site of illumination may move over time. In some embodiments, the discrete selected region of illumination may be a location within a sequestration pen of a microfluidic device, particularly within a target region described herein. In various embodiments, the discrete selected region of illumination is located within an isolation region of a sequestration pen. In various embodiments, the discrete selected region of illumination is comprised within a target region of the sequestration pen.

Thermal targets. In some embodiments, a thermal target is a microfluidic feature of the microfluidic device which may be a separate feature designed for this purpose. Alternatively, in some embodiments, a thermal target may be a location within the microfluidic circuit to which optical illumination is applied. The thermal target is a passive microfluidic feature and does not include any self-activating resistors or electrical heaters. The passive nature of the thermal targets simplifies fabrication of the microfluidic device. For thermal targets including metal or microstructures, fabrication is much less complex than an active thermal target such as a resistor, as is described below. Active thermal targets such as resistors and the like must have fixed electrical connections and are fabricated in fixed positions, unlike the passive thermal targets of the present disclosure. When the thermal target is a selected location of the microfluidic circuit material or base, with no additional structural feature, the flexibility to create forces specifically and selectively where needed is particularly advantageous compared to fixed active thermal targets. In some embodiments, a thermal target is located within a target region defined by an in situ-generated piston.

Period of illumination. The step of illumination may be performed using any light source as described herein, and may be a coherent or a non-coherent light. The light may be structured or unstructured light. For simplicity, the following description refers to laser illumination, but the invention is not so limited.

In various embodiments, the step of illuminating the selected discrete site (e.g., a site within the displacement force generation region) may include illuminating the selected discrete site with a laser. The laser may irradiate with light having a wavelength in the region of about 450 nm to about 800 nm. The laser may have a current of about 0.5 amps, 0.7 amps, 0.9 amps, 1.1 amps, 1.4 amps, 1.6 amps, 1.6 amps, 2.0 amps, 2.2 amps, 2.5 amps, 2.7 amps, 3.0 amps, or any value therebetween.

The laser illumination may have incident power in the range of about 1 mW to about 1000 mW, about 100 mW to about 1000 mW, about 100 mW to about 800 mW, about 100 mW to about 600 mW, about 100 mW to about 500 mW, or any range or individual value therebetween.

In various embodiments, the step of illuminating the selected discrete site (e.g., a site within the displacement force generation region) with laser illumination may be performed for a period of time in a range of about 10 microsec to about 8000 millisec, and may be any value therebetween. In some other embodiments, the step of illuminating the selected discrete site (e.g., a site within the displacement force generation region) may be performed for a period of time in the range of about 100 millisec to about 3 minutes.

In various embodiments, the laser illumination may be directed to the selected discrete site (e.g., a site within the displacement force generation region) for about 50 millisec, 75 millisec, 100 millisec, 150 millisec, 250 millisec, 500 millisec, 750 millisec, or about 1000 millisec. In various embodiments, the laser illumination may be directed to the selected discrete site (e.g., a site within the displacement force generation region) for a period of time in a range of about 50 millisec to about 2000 millisec; about 50 millisec to about 1000 millisec; about 50 millisec to about 500 millisec; about 50 millisec to about 300 millisec; 100 millisec to about 1000 millisec; about 200 millisec to about 1000 millisec; about 200 millisec to about 700 millisec; about 300 millisec to about 600 millisec; or any value therebetween of any of the ranges. In other embodiments, the laser illumination may be directed to the selected discrete site (e.g., a site within the displacement force generation region) for a period of time in a range of about 1 millisec to about 200 millisec; about 1 millisec to about 150 millisec; about 1 millisec to about 100 millisec; about 1 millisec to about 50 millisec, about 1 millisec to about 30 millisec; about 25 millisec to about 200 millisec; about 25 millisec to about 100 millisec; about 25 millisec to about 75 millisec; about 50 millisec to about 200 millisec, about 50 millisec to about 125 millisec; about 50 millisec to about 90 millisec; or may be any value therebetween of any of the ranges. A period of illumination selected in one of these ranges may be sufficient to optically drive generation of a bubble which can contact a piston and/or a micro-object and thereby move and/or facilitate unpenning of the micro object.

In various other embodiments, the laser illumination may be directed to the selected discrete site (e.g., a site within the displacement force generation region) for a period of time in a range of about 500 millisec to about 3000 millisec; about 1000 millisec to about 2700 millisec; about 1000 millisec to about 2500 millisec; about 1000 millisec to about 2000 millisec; about 1000 millisec to about 1500 millisec; about 1300 millisec to about 3000 millisec; about 1300 millisec to about 2700 millisec; about 1300 millisec to about 2300 millisec; about 1300 millisec to about 2000 millisec; about 1300 millisec to about 1700 millisec; about 1500 millisec to about 3000 millisec; about 1500 millisec to about 2600 millisec; about 1500 millisec to about 2300 millisec; about 1500 millisec to about 2000 millisec; about 1700 millisec to about 3000 millisec; about 1700 millisec to about 2600 millisec; about 1700 millisec to about 2000 millisec; or any value therebetween. A period of illumination chosen in one of these ranges may be suitable for generating optically-driven shear flow or bubble flow contact force.

In yet other embodiments, the step of illuminating the selected discrete site (e.g., a site within the displacement force generation region) may be performed for about 10 microsec to about 200 millisec; about 10 microsec to about 100 millisec; about 10 microsec to about 1 millisec; about 10 microsec to about 1 millisec; about 10 microsec to about 500 microsec; about 50 microsec to about 1 millisec; about 50 microsec to about 500 microsec; about 50 microsec to about 300 microsec; about 1 millisec to about 200 millisec; about 1 millisec to about 150 millisec; about 1 millisec to about 100 millisec; about 1 millisec to about 50 millisec; about 1 millisec to about 30 millisec; about 25 millisec to about 200 millisec; about 25 millisec to about 100 millisec; about 25 millisec to about 75 millisec; about 50 millisec to about 200 millisec; about 50 millisec to about 125 millisec; about 50 millisec to about 90 millisec; or may be any value therebetween of any of the ranges. A period of illumination in one such range of illumination may be sufficient to create a cavitating force within a discrete select site (e.g., a site within the displacement force generation region) adjacent to micro-objects, thereby dislodging one or more of the micro-objects, thus facilitating future unpenning. In some embodiments, the period of time of illumination may be in a range from about 10 microsec to about 500 microsec or from about 10 microsec to about 100 millisec.

In some other embodiments, the step of illuminating the selected discrete site (e.g., a site within the displacement force generation region) may be performed for about 100 millisec to about 3 minutes; about 100 millisec to about 2 minutes; about 100 millisec to about 1 minute; about 100 millisec to about 10,000 millisec; about 100 millisec to about 5,000 millisec; about 100 millisec to about 1000 millisec; about 500 millisec to about 3 minutes; about 500 millisec to about 1 minute; about 500 millisec to about 10,000 millisec, about 500 millisec to about 3,000 millisec; or any value therebetween.

These ranges are exemplary only and are not intended to limit the disclosure. Periods of illumination outside of the ranges described for each type of convective flow or displacement force may be identified and used while still remaining within the scope of the disclosure.

Kit. In light of the foregoing, a kit for performing methods of the present disclosure can be provided. In some embodiments, the kit comprises a flowable polymer, e.g., a prepolymer configured to be controllably activated to form at least one in situ-generated barrier that is a piston comprising a solidified polymer network, and optionally additional prepolymer configured to be controllably activated to form one or more additional in situ-generated barrier(s) comprising a solidified polymer network, wherein the in situ-generated barriers have a porosity that substantially prevents the cell from crossing through the in situ-generated barriers. The kit may further include instructions regarding methods of creating shapes suitable for in situ-generated barrier formation, and chamber locations appropriate for generation of said shapes. The kit may further include an inhibitor. The inhibitor may be packaged integrally with the flowable polymer, or may be packaged separately. In some other embodiments, the kit may include a first instance of the inhibitor packaged integrally with the flowable polymer and a second instance of the inhibitor packaged separately. In some other embodiments, the kit may further include an initiator, such as a photoinitiator suitable for inducing and/or catalyzing polymerization of a prepolymer.

In some embodiments, the kit further comprises a microfluidic device comprising a microfluidic circuit comprising a flow region and a chamber, wherein the chamber comprises an opening to the flow region. The microfluidic device can be any microfluidic device as described herein.

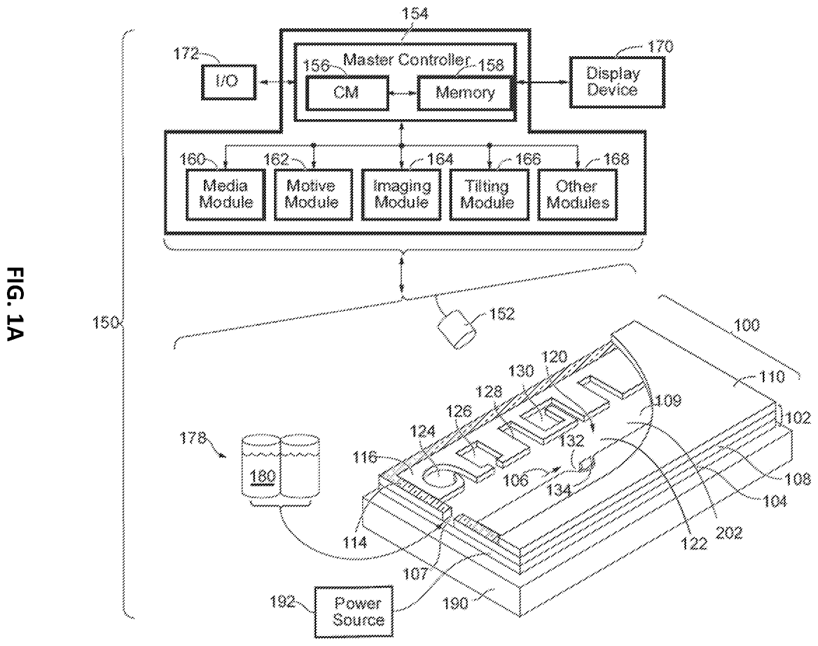

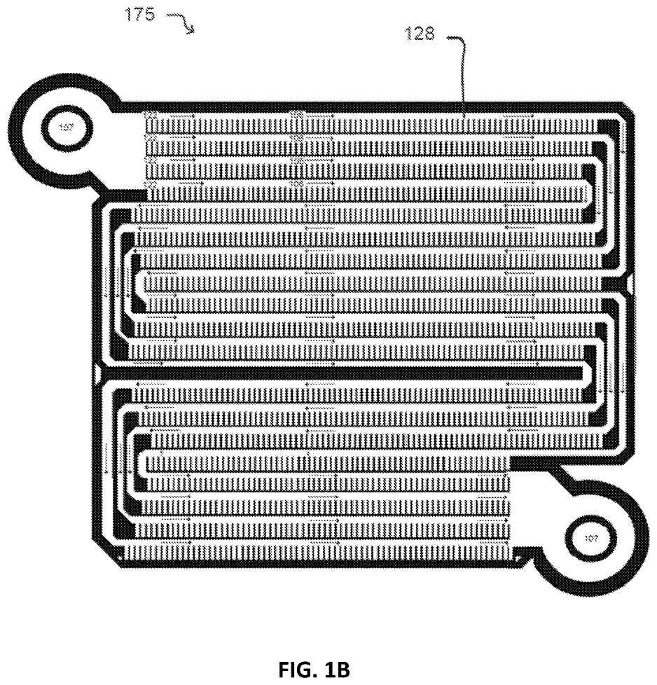

Microfluidic device/system feature cross-applicability. It should be appreciated that various features of microfluidic devices, systems, and motive technologies described herein may be combinable or interchangeable. For example, features described herein with reference to the microfluidic device 100, 175, 200, 300, 320, 400, 450, 520 and system attributes as described in FIGS. 1A-5B may be combinable or interchangeable.

Microfluidic devices. FIG. 1A illustrates an example of a microfluidic device 100. A perspective view of the microfluidic device 100 is shown having a partial cut-away of its cover 110 to provide a partial view into the microfluidic device 100. The microfluidic device 100 generally comprises a microfluidic circuit 120 comprising a flow path 106 through which a fluidic medium 180 can flow, optionally carrying one or more micro-objects (not shown) into and/or through the microfluidic circuit 120.

As generally illustrated in FIG. 1A, the microfluidic circuit 120 is defined by an enclosure 102. Although the enclosure 102 can be physically structured in different configurations, in the example shown in FIG. 1A the enclosure 102 is depicted as comprising a support structure 104 (e.g., a base), a microfluidic circuit structure 108, and a cover 110. The support structure 104, microfluidic circuit structure 108, and cover 110 can be attached to each other. For example, the microfluidic circuit structure 108 can be disposed on an inner surface 109 of the support structure 104, and the cover 110 can be disposed over the microfluidic circuit structure 108. Together with the support structure 104 and cover 110, the microfluidic circuit structure 108 can define the elements of the microfluidic circuit 120, forming a three-layer structure.

The support structure 104 can be at the bottom and the cover 110 at the top of the microfluidic circuit 120 as illustrated in FIG. 1A. Alternatively, the support structure 104 and the cover 110 can be configured in other orientations. For example, the support structure 104 can be at the top and the cover 110 at the bottom of the microfluidic circuit 120. Regardless, there can be one or more ports 107 each comprising a passage into or out of the enclosure 102. Examples of a passage include a valve, a gate, a pass-through hole, or the like. As illustrated, port 107 is a pass-through hole created by a gap in the microfluidic circuit structure 108. However, the port 107 can be situated in other components of the enclosure 102, such as the cover 110. Only one port 107 is illustrated in FIG. 1A but the microfluidic circuit 120 can have two or more ports 107. For example, there can be a first port 107 that functions as an inlet for fluid entering the microfluidic circuit 120, and there can be a second port 107 that functions as an outlet for fluid exiting the microfluidic circuit 120. Whether a port 107 function as an inlet or an outlet can depend upon the direction that fluid flows through flow path 106.

The support structure 104 can comprise one or more electrodes (not shown) and a substrate or a plurality of interconnected substrates. For example, the support structure 104 can comprise one or more semiconductor substrates, each of which is electrically connected to an electrode (e.g., all or a subset of the semiconductor substrates can be electrically connected to a single electrode). The support structure 104 can further comprise a printed circuit board assembly (“PCBA”). For example, the semiconductor substrate(s) can be mounted on a PCBA.

The microfluidic circuit structure 108 can define circuit elements of the microfluidic circuit 120. Such circuit elements can comprise spaces or regions that can be fluidly interconnected when microfluidic circuit 120 is filled with fluid, such as flow regions (which may include or be one or more flow channels), chambers (which class of circuit elements may also include sub-classes including sequestration pens), traps, and the like. Circuit elements can also include barriers, and the like. In the microfluidic circuit 120 illustrated in FIG. 1A, the microfluidic circuit structure 108 comprises a frame 114 and a microfluidic circuit material 116. The frame 114 can partially or completely enclose the microfluidic circuit material 116. The frame 114 can be, for example, a relatively rigid structure substantially surrounding the microfluidic circuit material 116. For example, the frame 114 can comprise a metal material. However, the microfluidic circuit structure need not include a frame 114. For example, the microfluidic circuit structure can consist of (or consist essentially of) the microfluidic circuit material 116.

The microfluidic circuit material 116 can be patterned with cavities or the like to define the circuit elements and interconnections of the microfluidic circuit 120, such as chambers, pens and microfluidic channels. The microfluidic circuit material 116 can comprise a flexible material, such as a flexible polymer (e.g., rubber, plastic, elastomer, silicone, polydimethylsiloxane (“PDMS”), or the like), which can be gas permeable. Other examples of materials that can form the microfluidic circuit material 116 include molded glass, an etchable material such as silicone (e.g., photo-patternable silicone or “PPS”), photoresist (e.g., SU8), or the like. In some embodiments, such materials—and thus the microfluidic circuit material 116—can be rigid and/or substantially impermeable to gas. Regardless, microfluidic circuit material 116 can be disposed on the support structure 104 and inside the frame 114.

The microfluidic circuit 120 can include a flow region in which one or more chambers can be disposed and/or fluidically connected thereto. A chamber can have one or more openings fluidically connecting the chamber with one or more flow regions. In some embodiments, a flow region comprises or corresponds to a microfluidic channel 122. Although a single microfluidic circuit 120 is illustrated in FIG. 1A, suitable microfluidic devices can include a plurality (e.g., 2 or 3) of such microfluidic circuits. In some embodiments, the microfluidic device 100 can be configured to be a nanofluidic device. As illustrated in FIG. 1A, the microfluidic circuit 120 may include a plurality of microfluidic sequestration pens 124, 126, 128, and 130, where each sequestration pens may have one or more openings. In some embodiments of sequestration pens, a sequestration pen may have only a single opening in fluidic communication with the flow path 106. In some other embodiments, a sequestration pen may have more than one opening in fluidic communication with the flow path 106, e.g., n number of openings, but with n−a1 openings that are valved, such that all but one opening is closable. When all the valved openings are closed, the sequestration pen limits exchange of materials from the flow region into the sequestration pen to occur only by diffusion. In some embodiments, the sequestration pens comprise various features and structures (e.g., isolation regions) that have been optimized for retaining micro-objects within the sequestration pen (and therefore within a microfluidic device such as microfluidic device 100) even when a medium 180 is flowing through the flow path 106.

The cover 110 can be an integral part of the frame 114 and/or the microfluidic circuit material 116. Alternatively, the cover 110 can be a structurally distinct element, as illustrated in FIG. 1A. The cover 110 can comprise the same or different materials than the frame 114 and/or the microfluidic circuit material 116. In some embodiments, the cover 110 can be an integral part of the microfluidic circuit material 116. Similarly, the support structure 104 can be a separate structure from the frame 114 or microfluidic circuit material 116 as illustrated, or an integral part of the frame 114 or microfluidic circuit material 116. Likewise, the frame 114 and microfluidic circuit material 116 can be separate structures as shown in FIG. 1A or integral portions of the same structure. Regardless of the various possible integrations, the microfluidic device can retain a three-layer structure that includes a base layer and a cover layer that sandwich a middle layer in which the microfluidic circuit 120 is located.

In some embodiments, the cover 110 can comprise a rigid material. The rigid material may be glass or a material with similar properties. In some embodiments, the cover 110 can comprise a deformable material. The deformable material can be a polymer, such as PDMS. In some embodiments, the cover 110 can comprise both rigid and deformable materials. For example, one or more portions of cover 110 (e.g., one or more portions positioned over sequestration pens 124, 126, 128, 130) can comprise a deformable material that interfaces with rigid materials of the cover 110. Microfluidic devices having covers that include both rigid and deformable materials have been described, for example, in U.S. Pat. No. 10,058,865 (Breinlinger et al.), the contents of which are incorporated herein by reference. In some embodiments, the cover 110 can further include one or more electrodes. The one or more electrodes can comprise a conductive oxide, such as indium-tin-oxide (ITO), which may be coated on glass or a similarly insulating material. Alternatively, the one or more electrodes can be flexible electrodes, such as single-walled nanotubes, multi-walled nanotubes, nanowires, clusters of electrically conductive nanoparticles, or combinations thereof, embedded in a deformable material, such as a polymer (e.g., PDMS). Flexible electrodes that can be used in microfluidic devices have been described, for example, in U.S. Pat. No. 9,227,200 (Chiou et al.), the contents of which are incorporated herein by reference. In some embodiments, the cover 110 and/or the support structure 104 can be transparent to light. The cover 110 may also include at least one material that is gas permeable (e.g., PDMS or PPS).

In the example shown in FIG. 1A, the microfluidic circuit 120 is illustrated as comprising a microfluidic channel 122 and sequestration pens 124, 126, 128, 130. Each pen comprises an opening to channel 122, but otherwise is enclosed such that the pens can substantially isolate micro-objects inside the pen from fluidic medium 180 and/or micro-objects in the flow path 106 of channel 122 or in other pens. The walls of the sequestration pen extend from the inner surface 109 of the base to the inside surface of the cover 110 to provide enclosure. The opening of the sequestration pen to the microfluidic channel 122 is oriented at an angle to the flow 106 of fluidic medium 180 such that flow 106 is not directed into the pens. The vector of bulk fluid flow in channel 122 may be tangential or parallel to the plane of the opening of the sequestration pen, and is not directed into the opening of the pen. In some instances, pens 124, 126, 128, 130 are configured to physically isolate one or more micro-objects within the microfluidic circuit 120. Sequestration pens in accordance with the present disclosure can comprise various shapes, surfaces and features that are optimized for use with DEP, OET, OEW, fluid flow, magnetic forces, centripetal, and/or gravitational forces, as will be discussed and shown in detail below.

The microfluidic circuit 120 may comprise any number of microfluidic sequestration pens. Although five sequestration pens are shown, microfluidic circuit 120 may have fewer or more sequestration pens. As shown, microfluidic sequestration pens 124, 126, 128, and 130 of microfluidic circuit 120 each comprise differing features and shapes which may provide one or more benefits useful for maintaining, isolating, assaying or culturing biological micro-objects. In some embodiments, the microfluidic circuit 120 comprises a plurality of identical microfluidic sequestration pens.

In the embodiment illustrated in FIG. 1A, a single flow path 106 containing a single channel 122 is shown. However, other embodiments may contain multiple channels 122 within a single flow path 106, as shown in FIG. 1B. The microfluidic circuit 120 further comprises an inlet valve or port 107 in fluid communication with the flow path 106, whereby fluidic medium 180 can access the flow path 106 (and channel 122). In some instances, the flow path 106 comprises a substantially straight path. In other instances, the flow path 106 is arranged in a non-linear or winding manner, such as a zigzag pattern, whereby the flow path 106 travels across the microfluidic device 100 two or more times, e.g., in alternating directions. The flow in the flow path 106 may proceed from inlet to outlet or may be reversed and proceed from outlet to inlet.

One example of a multi-channel device, microfluidic device 175, is shown in FIG. 1B, which may be like microfluidic device 100 in other respects. Microfluidic device 175 and its constituent circuit elements (e.g., channels 122 and sequestration pens 128) may have any of the dimensions discussed herein. The microfluidic circuit illustrated in FIG. 1B has two inlet/outlet ports 107 and a flow path 106 containing four distinct channels 122. The number of channels into which the microfluidic circuit is sub-divided may be chosen to reduce fluidic resistance. For example, the microfluidic circuit may include 1, 2, 3, 4, 5, 6, 7, 8, 9, 10, or more channels to provide a selected range of fluidic resistance. Microfluidic device 175 further comprises a plurality of sequestration pens opening off of each channel 122, where each of the sequestration pens is similar to sequestration pen 128 of FIG. 1A, and may have any of the dimensions or functions of any sequestration pen as described herein. However, the sequestration pens of microfluidic device 175 can have different shapes, such as any of the shapes of sequestration pens 124, 126, or 130 of FIG. 1A or as described anywhere else herein. Moreover, microfluidic device 175 can include sequestration pens having a mixture of different shapes. In some instances, a plurality of sequestration pens is configured (e.g., relative to a channel 122) such that the sequestration pens can be loaded with target micro-objects in parallel.

Returning to FIG. 1A, microfluidic circuit 120 further may include one or more optional micro-object traps 132. The optional traps 132 may be formed in a wall forming the boundary of a channel 122, and may be positioned opposite an opening of one or more of the microfluidic sequestration pens 124, 126, 128, 130. The optional traps 132 may be configured to receive or capture a single micro-object from the flow path 106, or may be configured to receive or capture a plurality of micro-objects from the flow path 106. In some instances, the optional traps 132 comprise a volume approximately equal to the volume of a single target micro-object. In some instances, the trap 132 comprises a side passage 134 that is smaller than the target micro-object in order to facilitate flow through the trap 132.

Sequestration pens. The microfluidic devices described herein may include one or more sequestration pens, where each sequestration pen is suitable for holding one or more micro-objects (e.g., biological cells, or groups of cells that are associated together). The sequestration pens may be disposed within and open to a flow region, which in some embodiments is a microfluidic channel. Each of the sequestration pens can have one or more openings for fluidic communication to one or more microfluidic channels. In some embodiments, a sequestration pen may have only one opening to a microfluidic channel.

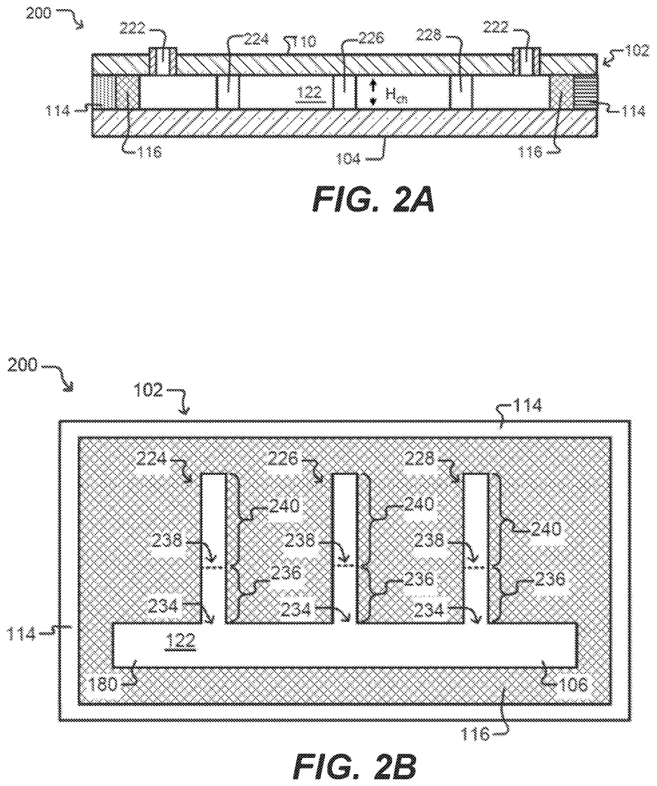

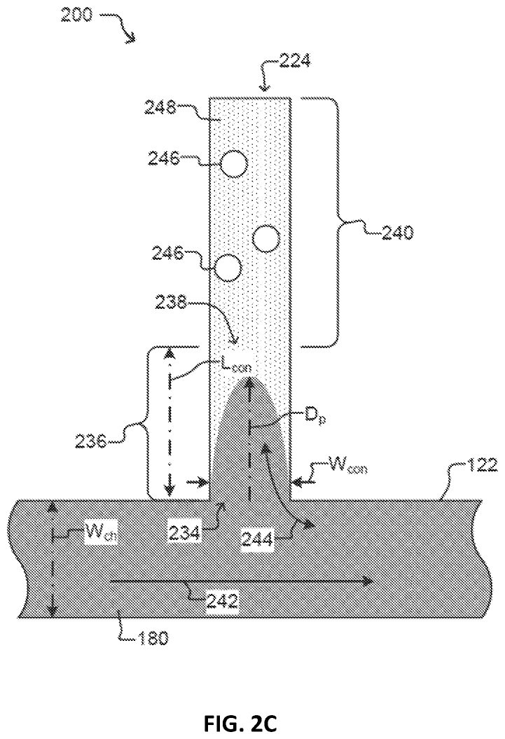

FIGS. 2A-2C show sequestration pens 224, 226, and 228 of a microfluidic device 200, which may be like sequestration pen 128 of FIG. 1A. Each sequestration pen 224, 226, and 228 can comprise an isolation region 240 and a connection region 236 fluidically connecting the isolation region 240 to a flow region, which may, in some embodiments include a microfluidic channel, such as channel 122. The connection region 236 can comprise a proximal opening 234 to the flow region (e.g., microfluidic channel 122) and a distal opening 238 to the isolation region 240. The connection region 236 can be configured so that the maximum penetration depth of a flow of a fluidic medium (not shown) flowing in the microfluidic channel 122 past the sequestration pen 224, 226, and 228 does not extend into the isolation region 240, as discussed below for FIG. 2C. In some embodiments, streamlines from the flow in the microfluidic channel do not enter the isolation region. Thus, due to the connection region 236, a micro-object (not shown) or other material (not shown) disposed in the isolation region 240 of a sequestration pen 224, 226, and 228 can be isolated from, and not substantially affected by, a flow of fluidic medium 180 in the microfluidic channel 122.

The sequestration pens 224, 226, and 228 of FIGS. 2A-2C each have a single opening which opens directly to the microfluidic channel 122. The opening of the sequestration pen may open laterally from the microfluidic channel 122, as shown in FIG. 2A, which depicts a vertical cross-section of microfluidic device 200. FIG. 2B shows a horizontal cross-section of microfluidic device 200. An electrode activation substrate 206 can underlie both the microfluidic channel 122 and the sequestration pens 224, 226, and 228. The upper surface of the electrode activation substrate 206 within an enclosure of a sequestration pen, forming the floor of the sequestration pen, can be disposed at the same level or substantially the same level of the upper surface the of electrode activation substrate 206 within the microfluidic channel 122 (or flow region if a channel is not present), forming the floor of the flow channel (or flow region, respectively) of the microfluidic device. The electrode activation substrate 206 may be featureless or may have an irregular or patterned surface that varies from its highest elevation to its lowest depression by less than about 3 micrometers (microns), 2.5 microns, 2 microns, 1.5 microns, 1 micron, 0.9 microns, 0.5 microns, 0.4 microns, 0.2 microns, 0.1 microns or less. The variation of elevation in the upper surface of the substrate across both the microfluidic channel 122 (or flow region) and sequestration pens may be equal to or less than about 10%, 7%, 5%, 3%, 2%, 1%, 0.9%, 0.8%, 0.5%, 0.3% or 0.1% of the height of the walls of the sequestration pen. Alternatively, the variation of elevation in the upper surface of the substrate across both the microfluidic channel 122 (or flow region) and sequestration pens may be equal to or less than about 2%, 1%, 0.9%, 0.8%, 0.5%, 0.3%, 0.2%, or 0.1% of the height of the substrate. While described in detail for the microfluidic device 200, this may also apply to any of the microfluidic devices described herein.

The microfluidic channel 122 and connection region 236 can be examples of swept regions, and the isolation regions 240 of the sequestration pens 224, 226, and 228 can be examples of unswept regions. Sequestration pens like 224, 226, 228 have isolation regions wherein each isolation region has only one opening, which opens to the connection region of the sequestration pen. Fluidic media exchange in and out of the isolation region so configured can be limited to occurring substantially only by diffusion. As noted, the microfluidic channel 122 and sequestration pens 224, 226, and 228 can be configured to contain one or more fluidic media 180. In the example shown in FIGS. 2A-2B, ports 222 are connected to the microfluidic channel 122 and allow the fluidic medium 180 to be introduced into or removed from the microfluidic device 200. Prior to introduction of the fluidic medium 180, the microfluidic device may be primed with a gas such as carbon dioxide gas. Once the microfluidic device 200 contains the fluidic medium 180, the flow 242 (see FIG. 2C) of fluidic medium 180 in the microfluidic channel 122 can be selectively generated and stopped. For example, as shown, the ports 222 can be disposed at different locations (e.g., opposite ends) of the flow region (microfluidic channel 122), and a flow 242 of the fluidic medium can be created from one port 222 functioning as an inlet to another port 222 functioning as an outlet.

FIG. 2C illustrates a detailed view of an example of a sequestration pen 224, which may contain one or more micro-objects 246, according to some embodiments. The flow 242 of fluidic medium 180 in the microfluidic channel 122 past the proximal opening 234 of the connection region 236 of sequestration pen 224 can cause a secondary flow 244 of the fluidic medium 180 into and out of the sequestration pen 224. To sequester the micro-objects 246 in the isolation region 240 of the sequestration pen 224 from the secondary flow 244, the length L_{con} of the connection region 236 of the sequestration pen 224 (i.e., from the proximal opening 234 to the distal opening 238) should be greater than the penetration depth Dₚ of the secondary flow 244 into the connection region 236. The penetration depth Dₚ depends upon a number of factors, including the shape of the microfluidic channel 122, which may be defined by a width W_{con} of the connection region 236 at the proximal opening 234; a width W_{ch} of the microfluidic channel 122 at the proximal opening 234; a height H_{ch} of the channel 122 at the proximal opening 234; and the width of the distal opening 238 of the connection region 236. Of these factors, the width W_{con} of the connection region 236 at the proximal opening 234 and the height H_{ch} of the channel 122 at the proximal opening 234 tend to be the most significant. In addition, the penetration depth Dₚ can be influenced by the velocity of the fluidic medium 180 in the channel 122 and the viscosity of fluidic medium 180. However, these factors (i.e., velocity and viscosity) can vary widely without dramatic changes in penetration depth Dₚ. For example, for a microfluidic chip 200 having a width W_{con} of the connection region 236 at the proximal opening 234 of about 50 microns, a height H_{ch} of the channel 122 at the proximal opening 122 of about 40 microns, and a width W_{ch} of the microfluidic channel 122 at the proximal opening 122 of about 100 microns to about 150 microns, the penetration depth Dₚ of the secondary flow 244 ranges from less than 1.0 times W_{con} (i.e., less than 50 microns) at a flow rate of 0.1 microliters/sec to about 2.0 times W_{con} (i.e., about 100 microns) at a flow rate of 20 microliters/sec, which represents an increase in Dₚ of only about 2.5-fold over a 200-fold increase in the velocity of the fluidic medium 180.

In some embodiments, the walls of the microfluidic channel 122 and sequestration pen 224, 226, or 228 can be oriented as follows with respect to the vector of the flow 242 of fluidic medium 180 in the microfluidic channel 122: the microfluidic channel width W_{ch} (or cross-sectional area of the microfluidic channel 122) can be substantially perpendicular to the flow 242 of medium 180; the width W_{con} (or cross-sectional area) of the connection region 236 at opening 234 can be substantially parallel to the flow 242 of medium 180 in the microfluidic channel 122; and/or the length L_{con} of the connection region can be substantially perpendicular to the flow 242 of medium 180 in the microfluidic channel 122. The foregoing are examples only, and the relative position of the microfluidic channel 122 and sequestration pens 224, 226 and 228 can be in other orientations with respect to each other.

In some embodiments, for a given microfluidic device, the configurations of the microfluidic channel 122 and the opening 234 may be fixed, whereas the rate of flow 242 of fluidic medium 180 in the microfluidic channel 122 may be variable. Accordingly, for each sequestration pen 224, a maximal velocity Vₘₐₓ for the flow 242 of fluidic medium 180 in channel 122 may be identified that ensures that the penetration depth Dₚ of the secondary flow 244 does not exceed the length L_{con} of the connection region 236. When Vₘₐₓ is not exceeded, the resulting secondary flow 244 can be wholly contained within the connection region 236 and does not enter the isolation region 240. Thus, the flow 242 of fluidic medium 180 in the microfluidic channel 122 (swept region) is prevented from drawing micro-objects 246 out of the isolation region 240, which is an unswept region of the microfluidic circuit, resulting in the micro-objects 246 being retained within the isolation region 240. Accordingly, selection of microfluidic circuit element dimensions and further selection of the operating parameters (e.g., velocity of fluidic medium 180) can prevent contamination of the isolation region 240 of sequestration pen 224 by materials from the microfluidic channel 122 or another sequestration pen 226 or 228. It should be noted, however, that for many microfluidic chip configurations, there is no need to worry about Vₘₐₓ per se, because the chip will break from the pressure associated with flowing fluidic medium 180 at high velocity through the chip before Vₘₐₓ can be achieved.

Components (not shown) in the first fluidic medium 180 in the microfluidic channel 122 can mix with the second fluidic medium 248 in the isolation region 240 substantially only by diffusion of components of the first medium 180 from the microfluidic channel 122 through the connection region 236 and into the second fluidic medium 248 in the isolation region 240. Similarly, components (not shown) of the second medium 248 in the isolation region 240 can mix with the first medium 180 in the microfluidic channel 122 substantially only by diffusion of components of the second medium 248 from the isolation region 240 through the connection region 236 and into the first medium 180 in the microfluidic channel 122. In some embodiments, the extent of fluidic medium exchange between the isolation region of a sequestration pen and the flow region by diffusion is greater than about 90%, 91%, 92%, 93%, 94% 95%, 96%, 97%, 98%, or greater than about 99% of fluidic exchange.

In some embodiments, the first medium 180 can be the same medium or a different medium than the second medium 248. In some embodiments, the first medium 180 and the second medium 248 can start out being the same, then become different (e.g., through conditioning of the second medium 248 by one or more cells in the isolation region 240, or by changing the medium 180 flowing through the microfluidic channel 122).

As illustrated in FIG. 2C, the width W_{con} of the connection region 236 can be uniform from the proximal opening 234 to the distal opening 238. The width W_{con} of the connection region 236 at the distal opening 238 can be any of the values identified herein for the width W_{con} of the connection region 236 at the proximal opening 234. In some embodiments, the width of the isolation region 240 at the distal opening 238 can be substantially the same as the width W_{con} of the connection region 236 at the proximal opening 234. Alternatively, the width W_{con} of the connection region 236 at the distal opening 238 can be different (e.g., larger or smaller) than the width W_{con} of the connection region 236 at the proximal opening 234. In some embodiments, the width W_{con} of the connection region 236 may be narrowed or widened between the proximal opening 234 and distal opening 238. For example, the connection region 236 may be narrowed or widened between the proximal opening and the distal opening, using a variety of different geometries (e.g., chamfering the connection region, beveling the connection region). Further, any part or subpart of the connection region 236 may be narrowed or widened (e.g., a portion of the connection region adjacent to the proximal opening 234).

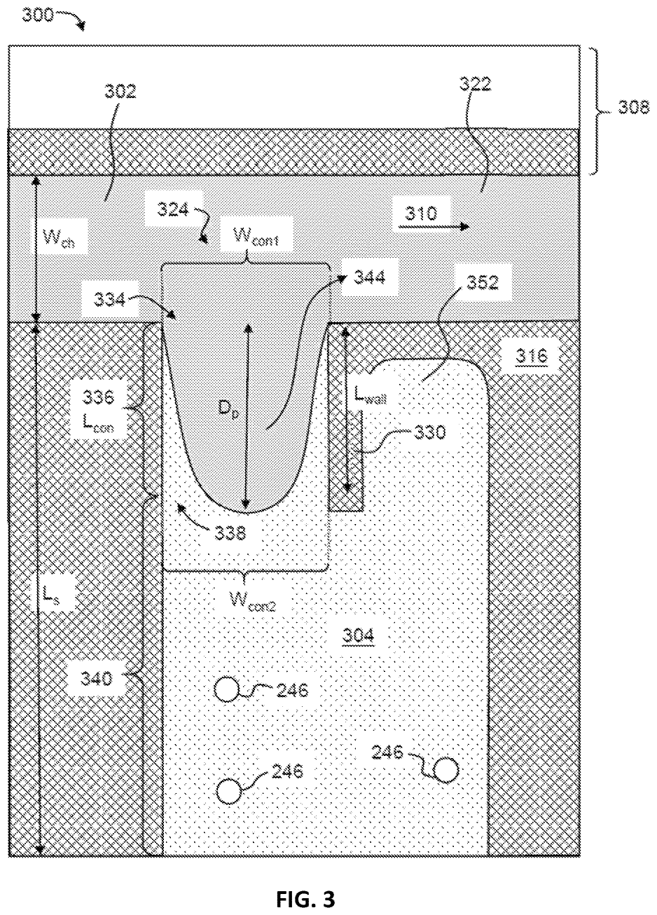

FIG. 3 depicts another exemplary embodiment of a microfluidic device 300 containing microfluidic circuit structure 308, which includes a channel 322 and sequestration pen 324, which has features and properties like any of the sequestration pens described herein for microfluidic devices 100, 175, 200, 400, 520 and any other microfluidic devices described herein.

The exemplary microfluidic devices of FIG. 3 include a microfluidic channel 322, having a width W_{ch}, as described herein, and containing a flow 310 of first fluidic medium 302 and one or more sequestration pens 324 (only one illustrated in FIG. 3). The sequestration pens 324 each have a length Lₛ, a connection region 336, and an isolation region 340, where the isolation region 340 contains a second fluidic medium 304. The connection region 336 has a proximal opening 334, having a width W_{con1}, which opens to the microfluidic channel 322, and a distal opening 338, having a width W_{con2}, which opens to the isolation region 340. The width W_{con1} may or may not be the same as W_{con2}, as described herein. The walls of each sequestration pen 324 may be formed of microfluidic circuit material 316, which may further form the connection region walls 330. A connection region wall 330 can correspond to a structure that is laterally positioned with respect to the proximal opening 334 and at least partially extends into the enclosed portion of the sequestration pen 324. In some embodiments, the length L_{con} of the connection region 336 is at least partially defined by length L_{wall} of the connection region wall 330. The connection region wall 330 may have a length L_{wall}, selected to be more than the penetration depth Dₚ of the secondary flow 344. Thus, the secondary flow 344 can be wholly contained within the connection region without extending into the isolation region 340.

The connection region wall 330 may define a hook region 352, which is a sub-region of the isolation region 340 of the sequestration pen 324. Since the connection region wall 330 extends into the inner cavity of the sequestration pen, the connection region wall 330 can act as a physical barrier to shield hook region 352 from secondary flow 344, with selection of the length of L_{wall}, contributing to the extent of the hook region. In some embodiments, the longer the length L_{wall} of the connection region wall 330, the more sheltered the hook region 352.

In sequestration pens configured like those of FIGS. 2A-2C and 3, the isolation region may have a shape and size of any type, and may be selected to regulate diffusion of nutrients, reagents, and/or media into the sequestration pen to reach to a far wall of the sequestration pen, e.g., opposite the proximal opening of the connection region to the flow region (or microfluidic channel). The size and shape of the isolation region may further be selected to regulate diffusion of waste products and/or secreted products of a biological micro-object out from the isolation region to the flow region via the proximal opening of the connection region of the sequestration pen. In general, the shape of the isolation region is not critical to the ability of the sequestration pen to isolate micro-objects from direct flow in the flow region.

In some other embodiments of sequestration pens, the isolation region may have more than one opening fluidically connecting the isolation region with the flow region of the microfluidic device. However, for an isolation region having a number of n openings fluidically connecting the isolation region to the flow region (or two or more flow regions), n−1 openings can be valved. When the n−1 valved openings are closed, the isolation region has only one effective opening, and exchange of materials into/out of the isolation region occurs only by diffusion.

Examples of microfluidic devices having pens in which biological micro-objects can be placed, cultured, and/or monitored have been described, for example, in U.S. Pat. No. 9,857,333 (Chapman, et al.), U.S. Pat. No. 10,010,882 (White, et al.), and U.S. Pat. No. 9,889,445 (Chapman, et al.), each of which is incorporated herein by reference in its entirety.

Microfluidic circuit element dimensions. Various dimensions and/or features of the sequestration pens and the microfluidic channels to which the sequestration pens open, as described herein, may be selected to limit introduction of contaminants or unwanted micro-objects into the isolation region of a sequestration pen from the flow region/microfluidic channel; limit the exchange of components in the fluidic medium from the channel or from the isolation region to substantially only diffusive exchange; facilitate the transfer of micro-objects into and/or out of the sequestration pens; and/or facilitate growth or expansion of the biological cells. Microfluidic channels and sequestration pens, for any of the embodiments described herein, may have any suitable combination of dimensions, may be selected by one of skill from the teachings of this disclosure.

For any of the microfluidic devices described herein, a microfluidic channel may have a uniform cross sectional height along its length that is a substantially uniform cross sectional height, and may be any cross sectional height as described herein. At any point along the microfluidic channel, the substantially uniform cross sectional height of the channel, the upper surface of which is defined by the inner surface of the cover and the lower surface of which is defined by the inner surface of the base, may be substantially the same as the cross sectional height at any other point along the channel, e.g., having a cross sectional height that is no more than about 10%, about 9%, about 8%, about 7%, about 6%, about 5%, about 4%, about 3%, about 2% or about 1% or less, different from the cross-sectional height of any other location within the channel.

Additionally, the chamber(s), e.g., sequestration pen(s), of the microfluidic devices described herein, may be disposed substantially in a coplanar orientation relative to the microfluidic channel into which the chamber(s) open. That is, the enclosed volume of the chamber(s) is formed by an upper surface that is defined by the inner surface of the cover, a lower surface defined by the inner surface of the base, and walls defined by the microfluidic circuit material. Therefore, the lower surface of the chamber(s) may be coplanar to the lower surface of the microfluidic channel, e.g., substantially coplanar. The upper surface of the chamber may be coplanar to the upper surface of the microfluidic channel, e.g., substantially coplanar. Accordingly, the chamber(s) may have a cross-sectional height, which may have any values as described herein, that is the same as the channel, e.g., substantially the same, and the chamber(s) and microfluidic channel(s) within the microfluidic device may have a substantially uniform cross sectional height throughout the flow region of the microfluidic device, and may be substantially coplanar throughout the microfluidic device.

Coplanarity of the lower surfaces of the chamber(s) and the microfluidic channel(s) can offer distinct advantage with repositioning micro-objects within the microfluidic device using DEP or magnetic force. Penning and unpenning of micro-objects, and in particular selective penning/selective unpenning, can be greatly facilitated when the lower surfaces of the chamber(s) and the microfluidic channel to which the chamber(s) open have a coplanar orientation.

The proximal opening of the connection region of a sequestration pen may have a width (e.g., W_{con} or W_{con1}) that is at least as large as the largest dimension of a micro-object (e.g., a biological cell, which may be a plant cell, such as a plant protoplast) for which the sequestration pen is intended. In some embodiments, the proximal opening has a width (e.g., W_{con} or W_{con1}) of about 20 microns, about 40 microns, about 50 microns, about 60 microns, about 75 microns, about 100 microns, about 150 microns, about 200 microns, or about 300 microns. The foregoing are examples only, and the width (e.g., W_{con} or W_{con1}) of a proximal opening can be selected to be a value between any of the values listed above (e.g., about 20-200 microns, about 20-150 microns, about 20-100 microns, about 20-75 microns, about 20-60 microns, about 50-300 microns, about 50-200 microns, about 50-150 microns, about 50-100 microns, about 50-75 microns, about 75-150 microns, about 75-100 microns, about 100-300 microns, about 100-200 microns, or about 200-300 microns).

In some embodiments, the connection region of the sequestration pen may have a length (e.g., L_{con}) from the proximal opening to the distal opening to the isolation region of the sequestration pen that is at least 0.5 times, at least 0.6 times, at least 0.7 times, at least 0.8 times, at least 0.9 times, at least 1.0 times, at least 1.1 times, at least 1.2 times, at least 1.3 times, at least 1.4 times, at least 1.5 times, at least 1.75 times, at least 2.0 times, at least 2.25 times, at least 2.5 times, at least 2.75 times, at least 3.0 times, at least 3.5 times, at least 4.0 times, at least 4.5 times, at least 5.0 times, at least 6.0 times, at least 7.0 times, at least 8.0 times, at least 9.0 times, or at least 10.0 times the width (e.g., W_{con} or W_{con1}) of the proximal opening. Thus, for example, the proximal opening of the connection region of a sequestration pen may have a width (e.g., W_{con} or W_{con1}) from about 20 microns to about 200 microns (e.g., about 50 microns to about 150 microns), and the connection region may have a length L_{con} that is at least 1.0 times (e.g., at least 1.5 times, or at least 2.0 times) the width of the proximal opening. As another example, the proximal opening of the connection region of a sequestration pen may have a width (e.g., W_{con} or W_{con1}) from about 20 microns to about 100 microns (e.g., about 20 microns to about 60 microns), and the connection region may have a length L_{con} that is at least 1.0 times (e.g., at least 1.5 times, or at least 2.0 times) the width of the proximal opening.

The microfluidic channel of a microfluidic device to which a sequestration pen opens may have specified size (e.g., width or height). In some embodiments, the height (e.g., H_{ch}) of the microfluidic channel at a proximal opening to the connection region of a sequestration pen can be within any of the following ranges: 20-100 microns, 20-90 microns, 20-80 microns, 20-70 microns, 20-60 microns, 20-50 microns, 30-100 microns, 30-90 microns, 30-80 microns, 30-70 microns, 30-60 microns, 30-50 microns, 40-100 microns, 40-90 microns, 40-80 microns, 40-70 microns, 40-60 microns, or 40-50 microns. The foregoing are examples only, and the height (e.g., H_{ch}) of the microfluidic channel (e.g., 122) can be selected to be between any of the values listed above. Moreover, the height (e.g., H_{ch}) of the microfluidic channel 122 can be selected to be any of these heights in regions of the microfluidic channel other than at a proximal opening of a sequestration pen.

The width (e.g., W_{ch}) of the microfluidic channel at the proximal opening to the connection region of a sequestration pen can be within any of the following ranges: about 20-500 microns, 20-400 microns, 20-300 microns, 20-200 microns, 20-150 microns, 20-100 microns, 20-80 microns, 20-60 microns, 30-400 microns, 30-300 microns, 30-200 microns, 30-150 microns, 30-100 microns, 30-80 microns, 30-60 microns, 40-300 microns, 40-200 microns, 40-150 microns, 40-100 microns, 40-80 microns, 40-60 microns, 50-1000 microns, 50-500 microns, 50-400 microns, 50-300 microns, 50-250 microns, 50-200 microns, 50-150 microns, 50-100 microns, 50-80 microns, 60-300 microns, 60-200 microns, 60-150 microns, 60-100 microns, 60-80 microns, 70-500 microns, 70-400 microns, 70-300 microns, 70-250 microns, 70-200 microns, 70-150 microns, 70-100 microns, 80-100 microns, 90-400 microns, 90-300 microns, 90-250 microns, 90-200 microns, 90-150 microns, 100-300 microns, 100-250 microns, 100-200 microns, 100-150 microns, 100-120 microns, 200-800 microns, 200-700 microns, or 200-600 microns. The foregoing are examples only, and the width (e.g., W_{ch}) of the microfluidic channel can be a value selected to be between any of the values listed above. Moreover, the width (e.g., W_{ch}) of the microfluidic channel can be selected to be in any of these widths in regions of the microfluidic channel other than at a proximal opening of a sequestration pen. In some embodiments, the width W_{ch} of the microfluidic channel at the proximal opening to the connection region of the sequestration pen (e.g., taken transverse to the direction of bulk flow of fluid through the channel) can be substantially perpendicular to a width (e.g., W_{con} or W_{con1}) of the proximal opening.

A cross-sectional area of the microfluidic channel at a proximal opening to the connection region of a sequestration pen can be about 500-50,000 square microns, 500-40,000 square microns, 500-30,000 square microns, 500-25,000 square microns, 500-20,000 square microns, 500-15,000 square microns, 500-10,000 square microns, 500-7,500 square microns, 500-5,000 square microns, 1,000-25,000 square microns, 1,000-20,000 square microns, 1,000-15,000 square microns, 1,000-10,000 square microns, 1,000-7,500 square microns, 1,000-5,000 square microns, 2,000-20,000 square microns, 2,000-15,000 square microns, 2,000-10,000 square microns, 2,000-7,500 square microns, 2,000-6,000 square microns, 3,000-20,000 square microns, 3,000-15,000 square microns, 3,000-10,000 square microns, 3,000-7,500 square microns, or 3,000 to 6,000 square microns. The foregoing are examples only, and the cross-sectional area of the microfluidic channel at the proximal opening can be selected to be between any of the values listed above. In various embodiments, and the cross-sectional area of the microfluidic channel at regions of the microfluidic channel other than at the proximal opening can also be selected to be between any of the values listed above. In some embodiments, the cross-sectional area is selected to be a substantially uniform value for the entire length of the microfluidic channel.

In some embodiments, the microfluidic chip is configured such that the proximal opening (e.g., 234 or 334) of the connection region of a sequestration pen may have a width (e.g., W_{con} or W_{con1}) from about 20 microns to about 200 microns (e.g., about 50 microns to about 150 microns), the connection region may have a length L_{con} (e.g., 236 or 336) that is at least 1.0 times (e.g., at least 1.5 times, or at least 2.0 times) the width of the proximal opening, and the microfluidic channel may have a height (e.g., H_{ch}) at the proximal opening of about 30 microns to about 60 microns. As another example, the proximal opening (e.g., 234 or 334) of the connection region of a sequestration pen may have a width (e.g., W_{con} or W_{con1}) from about 20 microns to about 100 microns (e.g., about 20 microns to about 60 microns), the connection region may have a length L_{con} (e.g., 236 or 336) that is at least 1.0 times (e.g., at least 1.5 times, or at least 2.0 times) the width of the proximal opening, and the microfluidic channel may have a height (e.g., H_{ch}) at the proximal opening of about 30 microns to about 60 microns. The foregoing are examples only, and the width (e.g., W_{con} or W_{con1}) of the proximal opening (e.g., 234 or 274), the length (e.g., L_{con}) of the connection region, and/or the width (e.g., W_{ch}) of the microfluidic channel (e.g., 122 or 322), can be a value selected to be between any of the values listed above. Generally, however, the width (W_{con} or W_{con1}) of the proximal opening of the connection region of a sequestration pen is less than the width (W_{ch}) of the microfluidic channel. In some embodiments, the width (W_{con} or W_{con1}) of the proximal opening is about 8%, 9%, 10%, 11%, 12%, 13%, 14%, 15%, 16%, 17%, 18%, 19%, 20%, 22%, 24%, 25%, or 30% of the width (W_{ch}) of the microfluidic channel. That is, the width (W_{ch}) of the microfluidic channel may be at least 2.5 times, 3.0 times, 3.5 times, 4.0 times, 4.5 times, 5.0 times, 6.0 times, 7.0 times, 8.0 times, 9.0 times or at least 10.0 times the width (W_{con} or W_{con1}) of the proximal opening of the connection region of the sequestration pen.

In some embodiments, the size Wc (e.g., cross-sectional width W_{ch}, diameter, area, or the like) of the channel 122, 322, 618, 718 can be about one and a quarter (1.25), about one and a half (1.5), about two, about two and a half (2.5), about three (3), or more times the size W_{O} (e.g., cross-sectional width W_{con}, diameter, area, or the like) of a chamber opening, e.g., sequestration pen opening 234, 334, and the like. This can reduce the extent of secondary flow and the rate of diffusion (or diffusion flux) through the opening 234, 334 for materials diffusing from a selected chamber (e.g., like sequestration pens 224, 226 of FIG. 2B) into channel 122, 322, 618, 718 and subsequently re-entering a downstream or adjacent chamber (e.g., like sequestration pen 228). The rate of diffusion of a molecule (e.g., an analyte of interest, such as an antibody) is dependent on a number of factors, including (without limitation) temperature, viscosity of the medium, and the coefficient of diffusion Do of the molecule. For example, the Do for an IgG antibody in aqueous solution at about 20 °C is about 4.4×10⁻⁷ cm²/sec, while the kinematic viscosity of cell culture medium is about 9×10⁻⁴ m²/sec. Thus, an antibody in cell culture medium at about 20 °C can have a rate of diffusion of about 0.5 microns/sec. Accordingly, in some embodiments, a time period for diffusion from a biological micro-object located within a sequestration pen such as 224, 226, 228, 324 into the channel 122, 322, 618, 718 can be about 10 minutes or less (e.g., about 9, 8, 7, 6, 5 minutes, or less). The time period for diffusion can be manipulated by changing parameters that influence the rate of diffusion. For example, the temperature of the media can be increased (e.g., to a physiological temperature such as about 37 °C) or decreased (e.g., to about 15 °C, 10 °C, or 4 °C) thereby increasing or decreasing the rate of diffusion, respectively. Alternatively, or in addition, the concentrations of solutes in the medium can be increased or decreased as discussed herein to isolate a selected pen from solutes from other upstream pens.

Accordingly, in some variations, the width (e.g., W_{ch}) of the microfluidic channel at the proximal opening to the connection region of a sequestration pen may be about 50 to 500 microns, about 50 to 300 microns, about 50 to 200 microns, about 70 to 500 microns, about to 70-300 microns, about 70 to 250 microns, about 70 to 200 microns, about 70 to 150 microns, about 70 to 100 microns, about 80 to 500 microns, about 80 to 300 microns, about 80 to 250 microns, about 80 to 200 microns, about 80 to 150 microns, about 90 to 500 microns, about 90 to 300 microns, about 90 to 250 microns, about 90 to 200 microns, about 90 to 150 microns, about 100 to 500 microns, about 100 to 300 microns, about 100 to 250 microns, about 100 to 200 microns, or about 100 to 150 microns. In some embodiments, the width W_{ch} of the microfluidic channel at the proximal opening to the connection region of a sequestration pen may be about 70 to 250 microns, about 80 to 200 microns, or about 90 to 150 microns. The width W_{con} of the opening of the chamber (e.g., sequestration pen) may be about 20 to 100 microns; about 30 to 90 microns; or about 20 to 60 microns. In some embodiments, W_{ch} is about 70-250 microns and W_{con} is about 20 to 100 microns; W_{ch} is about 80 to 200 microns and W_{con} is about 30 to 90 microns; W_{ch} is about 90 to 150 microns, and W_{con} is about 20 to 60 microns; or any combination of the widths of W_{ch} and W_{con} thereof.

In some embodiments, the proximal opening (e.g., 234 or 334) of the connection region of a sequestration pen has a width (e.g., W_{con} or W_{con1}) that is 2.0 times or less (e.g., 2.0, 1.9, 1.8, 1.5, 1.3, 1.0, 0.8, 0.5, or 0.1 times) the height (e.g., H_{ch}) of the flow region/microfluidic channel at the proximal opening, or has a value that lies within a range defined by any two of the foregoing values.

In some embodiments, the width W_{con1} of a proximal opening (e.g., 234 or 334) of a connection region of a sequestration pen may be the same as a width W_{con2} of the distal opening (e.g., 238 or 338) to the isolation region thereof. In some embodiments, the width W_{con1} of the proximal opening may be different than a width W_{con2} of the distal opening, and W_{con1} and/or W_{con2} may be selected from any of the values described for W_{con} or W_{con1}. In some embodiments, the walls (including a connection region wall) that define the proximal opening and distal opening may be substantially parallel with respect to each other. In some embodiments, the walls that define the proximal opening and distal opening may be selected to not be parallel with respect to each other.

The length (e.g., L_{con}) of the connection region can be about 1-600 microns, 5-550 microns, 10-500 microns, 15-400 microns, 20-300 microns, 20-500 microns, 40-400 microns, 60-300 microns, 80-200 microns, about 100-150 microns, about 20-300 microns, about 20-250 microns, about 20-200 microns, about 20-150 microns, about 20-100 microns, about 30-250 microns, about 30-200 microns, about 30-150 microns, about 30-100 microns, about 30-80 microns, about 30-50 microns, about 45-250 microns, about 45-200 microns, about 45-100 microns, about 45-80 microns, about 45-60 microns, about 60-200 microns, about 60-150 microns, about 60-100 microns or about 60-80 microns. The foregoing are examples only, and length (e.g., L_{con}) of a connection region can be selected to be a value that is between any of the values listed above.

The connection region wall of a sequestration pen may have a length (e.g., L_{wall}) that is at least 0.5 times, at least 0.6 times, at least 0.7 times, at least 0.8 times, at least 0.9 times, at least 1.0 times, at least 1.1 times, at least 1.2 times, at least 1.3 times, at least 1.4 times, at least 1.5 times, at least 1.75 times, at least 2.0 times, at least 2.25 times, at least 2.5 times, at least 2.75 times, at least 3.0 times, or at least 3.5 times the width (e.g., W_{con} or W_{con1}) of the proximal opening of the connection region of the sequestration pen. In some embodiments, the connection region wall may have a length L_{wall} of about 20-200 microns, about 20-150 microns, about 20-100 microns, about 20-80 microns, or about 20-50 microns. The foregoing are examples only, and a connection region wall may have a length L_{wall} selected to be between any of the values listed above.

A sequestration pen may have a length Lₛ of about 40-600 microns, about 40-500 microns, about 40-400 microns, about 40-300 microns, about 40-200 microns, about 40-100 microns or about 40-80 microns. The foregoing are examples only, and a sequestration pen may have a length Lₛ selected to be between any of the values listed above.

According to some embodiments, a sequestration pen may have a specified height (e.g., Hₛ). In some embodiments, a sequestration pen has a height Hₛ of about 20 microns to about 200 microns (e.g., about 20 microns to about 150 microns, about 20 microns to about 100 microns, about 20 microns to about 60 microns, about 30 microns to about 150 microns, about 30 microns to about 100 microns, about 30 microns to about 60 microns, about 40 microns to about 150 microns, about 40 microns to about 100 microns, or about 40 microns to about 60 microns). The foregoing are examples only, and a sequestration pen can have a height Hₛ selected to be between any of the values listed above.

The height H_{con} of a connection region at a proximal opening of a sequestration pen can be a height within any of the following heights: 20-100 microns, 20-90 microns, 20-80 microns, 20-70 microns, 20-60 microns, 20-50 microns, 30-100 microns, 30-90 microns, 30-80 microns, 30-70 microns, 30-60 microns, 30-50 microns, 40-100 microns, 40-90 microns, 40-80 microns, 40-70 microns, 40-60 microns, or 40-50 microns. The foregoing are examples only, and the height H_{con} of the connection region can be selected to be between any of the values listed above. Typically, the height H_{con} of the connection region is selected to be the same as the height H_{ch} of the microfluidic channel at the proximal opening of the connection region. Additionally, the height Hₛ of the sequestration pen is typically selected to be the same as the height H_{con} of a connection region and/or the height H_{ch} of the microfluidic channel. In some embodiments, Hₛ, H_{con}, and H_{ch} may be selected to be the same value of any of the values listed above for a selected microfluidic device.

The isolation region can be configured to contain only one, two, three, four, five, or a similar relatively small number of micro-objects. In other embodiments, the isolation region may contain more than 10, more than 50 or more than 100 micro-objects. Accordingly, the volume of an isolation region can be, for example, at least 1×10⁴, 1×10⁵, 5×10⁵, 8×10⁵, 1×10⁶, 2×10⁶, 4×10⁶, 6×10⁶, 1×10⁷, 3×10⁷, 5×10⁷ 1×10⁸, 5×10⁸, or 8×10⁸ cubic microns, or more. The foregoing are examples only, and the isolation region can be configured to contain numbers of micro-objects and volumes selected to be between any of the values listed above (e.g., a volume between 1×10⁵ cubic microns and 5×10⁵ cubic microns, between 5×10⁵ cubic microns and 1×10⁶ cubic microns, between 1×10⁶ cubic microns and 2×10⁶ cubic microns, or between 2×10⁶ cubic microns and 1×10⁷ cubic microns).

According to some embodiments, a sequestration pen of a microfluidic device may have a specified volume. The specified volume of the sequestration pen (or the isolation region of the sequestration pen) may be selected such that a single cell or a small number of cells (e.g., 2-10 or 2-5) can rapidly condition the medium and thereby attain favorable (or optimal) growth conditions. In some embodiments, the sequestration pen has a volume of about 5×10⁵, 6×10⁵, 8×10⁵, 1×10⁶, 2×10⁶, 4×10⁶, 8×10⁶, 1×10⁷, 3×10⁷, 5×10⁷, or about 8×10⁷ cubic microns, or more. In some embodiments, the sequestration pen has a volume of about 1 nanoliter to about 50 nanoliters, 2 nanoliters to about 25 nanoliters, 2 nanoliters to about 20 nanoliters, about 2 nanoliters to about 15 nanoliters, or about 2 nanoliters to about 10 nanoliters. The foregoing are examples only, and a sequestration pen can have a volume selected to be any value that is between any of the values listed above.

According to some embodiments, the flow of fluidic medium within the microfluidic channel (e.g., 122 or 322) may have a specified maximum velocity (e.g., Vₘₐₓ). In some embodiments, the maximum velocity (e.g., Vₘₐₓ) may be set at around 0.2, 0.5, 0.7, 1.0, 1.3, 1.5, 2.0, 2.5, 3.0, 3.5, 4.0, 4.5, 5.0, 5.5, 6.0, 6.7, 7.0, 7.5, 8.0, 8.5, 9.0, 10, 11, 12, 13, 14, 15, 16, 17, 18, 19, 20, 22, 24, or 25 microliters/sec. The foregoing are examples only, and the flow of fluidic medium within the microfluidic channel can have a maximum velocity (e.g., Vₘₐₓ) selected to be a value between any of the values listed above. The flow of fluidic medium within the microfluidic channel typically may be flowed at a rate less than the Vₘₐₓ. While the Vₘₐₓ may vary depending on the specific size and numbers of channel and sequestration pens opening thereto, a fluidic medium may be flowed at about 0.1 microliters/sec to about 20 microliters/sec; about 0.1 microliters/sec to about 15 microliters/sec; about 0.1 microliters/sec to about 12 microliters/sec, about 0.1 microliters/sec to about 10 microliters/sec; about 0.1 microliter/sec to about 7 microliters/sec without exceeding the Vₘₐₓ. In some portions of a typical workflow, a flow rate of a fluidic medium may be about 0.1 microliters/sec; about 0.5 microliters/sec; about 1.0 microliters/sec; about 2.0 microliters/sec; about 3.0 microliters/sec; about 4.0 microliters/sec; about 5.0 microliters/sec; about 6.0 microliters/sec; about 7.0 microliters/sec; about 8.0 microliters/sec; about 9.0 microliters/sec; about 10.0 microliters/sec; about 11.0 microliters/sec; or any range defined by two of the foregoing values, e.g., 1-5 microliters/sec or 5-10 microliters/sec. The flow rate of a fluidic medium in the microfluidic channel may be equal to or less than about 12 microliters/sec; about 10 microliters/sec; about 8 microliters/sec, or about 6 microliters/sec.

In various embodiment, the microfluidic device has sequestration pens configured as in any of the embodiments discussed herein where the microfluidic device has about 5 to about 10 sequestration pens, about 10 to about 50 sequestration pens, about 25 to about 200 sequestration pens, about 100 to about 500 sequestration pens, about 200 to about 1000 sequestration pens, about 500 to about 1500 sequestration pens, about 1000 to about 2500 sequestration pens, about 2000 to about 5000 sequestration pens, about 3500 to about 7000 sequestration pens, about 5000 to about 10,000 sequestration pens, about 7,500 to about 15,000 sequestration pens, about 12,500 to about 20,000 sequestration pens, about 15,000 to about 25,000 sequestration pens, about 20,000 to about 30,000 sequestration pens, about 25,000 to about 35,000 sequestration pens, about 30,000 to about 40,000 sequestration pens, about 35,000 to about 45,000 sequestration pens, or about 40,000 to about 50,000 sequestration pens. The sequestration pens need not all be the same size and may include a variety of configurations (e.g., different widths, different features within the sequestration pen).

Coating solutions and coating agents. In some embodiments, at least one inner surface of the microfluidic device includes a coating material that provides a layer of organic and/or hydrophilic molecules suitable for maintenance, expansion and/or movement of biological micro-object(s) (i.e., the biological micro-object exhibits increased viability, greater expansion and/or greater portability within the microfluidic device). The conditioned surface may reduce surface fouling, participate in providing a layer of hydration, and/or otherwise shield the biological micro-objects from contact with the non-organic materials of the microfluidic device interior.

In some embodiments, substantially all the inner surfaces of the microfluidic device include the coating material. The coated inner surface(s) may include the surface of a flow region (e.g., channel), chamber, or sequestration pen, or a combination thereof. In some embodiments, each of a plurality of sequestration pens has at least one inner surface coated with coating materials. In other embodiments, each of a plurality of flow regions or channels has at least one inner surface coated with coating materials. In some embodiments, at least one inner surface of each of a plurality of sequestration pens and each of a plurality of channels is coated with coating materials. The coating may be applied before or after introduction of biological micro-object(s), or may be introduced concurrently with the biological micro-object(s). In some embodiments, the biological micro-object(s) may be imported into the microfluidic device in a fluidic medium that includes one or more coating agents. In other embodiments, the inner surface(s) of the microfluidic device (e.g., a microfluidic device having an electrode activation substrate such as, but not limited to, a device including dielectrophoresis (DEP) electrodes) may be treated or “primed” with a coating solution comprising a coating agent prior to introduction of the biological micro-object(s) into the microfluidic device. Any convenient coating agent/coating solution can be used, including but not limited to: serum or serum factors, bovine serum albumin (BSA), polymers, detergents, enzymes, and any combination thereof.

Synthetic polymer-based coating materials. The at least one inner surface may include a coating material that comprises a polymer. The polymer may be non-covalently bound (e.g., it may be non-specifically adhered) to the at least one surface. The polymer may have a variety of structural motifs, such as found in block polymers (and copolymers), star polymers (star copolymers), and graft or comb polymers (graft copolymers), all of which may be suitable for the methods disclosed herein. A wide variety of alkylene ether containing polymers may be suitable for use in the microfluidic devices described herein, including but not limited to Pluronic® polymers such as Pluronic® L44, L64, P85, and F127 (including F127NF). Other examples of suitable coating materials are described in US2016/0312165, the contents of which are herein incorporated by reference in their entirety.

Covalently linked coating materials. In some embodiments, the at least one inner surface includes covalently linked molecules that provide a layer of organic and/or hydrophilic molecules suitable for maintenance/expansion of biological micro-object(s) within the microfluidic device, providing a conditioned surface for such cells. The covalently linked molecules include a linking group, wherein the linking group is covalently linked to one or more surfaces of the microfluidic device, as described below. The linking group is also covalently linked to a surface modifying moiety configured to provide a layer of organic and/or hydrophilic molecules suitable for maintenance/expansion/movement of biological micro-object(s).

In some embodiments, the covalently linked moiety configured to provide a layer of organic and/or hydrophilic molecules suitable for maintenance/expansion of biological micro-object(s) may include alkyl or fluoroalkyl (which includes perfluoroalkyl) moieties; mono- or polysaccharides (which may include but is not limited to dextran); alcohols (including but not limited to propargyl alcohol); polyalcohols, including but not limited to polyvinyl alcohol; alkylene ethers, including but not limited to polyethylene glycol; polyelectrolytes (including but not limited to polyacrylic acid or polyvinyl phosphonic acid); amino groups (including derivatives thereof, such as, but not limited to alkylated amines, hydroxyalkylated amino group, guanidinium, and heterocyclic groups containing an unaromatized nitrogen ring atom, such as, but not limited to morpholinyl or piperazinyl); carboxylic acids including but not limited to propiolic acid (which may provide a carboxylate anionic surface); phosphonic acids, including but not limited to ethynyl phosphonic acid (which may provide a phosphonate anionic surface); sulfonate anions; carboxybetaines; sulfobetaines; sulfamic acids; or amino acids.

In various embodiments, the covalently linked moiety configured to provide a layer of organic and/or hydrophilic molecules suitable for maintenance/expansion of biological micro-object(s) in the microfluidic device may include non-polymeric moieties such as an alkyl moiety, amino acid moiety, alcohol moiety, amino moiety, carboxylic acid moiety, phosphonic acid moiety, sulfonic acid moiety, sulfamic acid moiety, or saccharide moiety. Alternatively, the covalently linked moiety may include polymeric moieties, which may include any of these moieties.

In some embodiments, a microfluidic device may have a hydrophobic layer upon the inner surface of the base which includes a covalently linked alkyl moiety. The covalently linked alkyl moiety may comprise carbon atoms forming a linear chain (e.g., a linear chain of at least 10 carbons, or at least 14, 16, 18, 20, 22, or more carbons) and may be an unbranched alkyl moiety. In some embodiments, the alkyl group may include a substituted alkyl group (e.g., some of the carbons in the alkyl group can be fluorinated or perfluorinated). In some embodiments, the alkyl group may include a first segment, which may include a perfluoroalkyl group, joined to a second segment, which may include a non-substituted alkyl group, where the first and second segments may be joined directly or indirectly (e.g., by means of an ether linkage). The first segment of the alkyl group may be located distal to the linking group, and the second segment of the alkyl group may be located proximal to the linking group.

In other embodiments, the covalently linked moiety may include at least one amino acid, which may include more than one type of amino acid. Thus, the covalently linked moiety may include a peptide or a protein. In some embodiments, the covalently linked moiety may include an amino acid which may provide a zwitterionic surface to support cell growth, viability, portability, or any combination thereof.

In other embodiments, the covalently linked moiety may further include a streptavidin or biotin moiety. In some embodiments, a modified biological moiety such as, for example, a biotinylated protein or peptide may be introduced to the inner surface of a microfluidic device bearing covalently linked streptavidin, and couple via the covalently linked streptavidin to the surface, thereby providing a modified surface presenting the protein or peptide.

In other embodiments, the covalently linked moiety may include at least one alkylene oxide moiety and may include any alkylene oxide polymer as described above. One useful class of alkylene ether containing polymers is polyethylene glycol (PEG Mw<100,000 Da) or alternatively polyethylene oxide (PEO, Mw>100,000). In some embodiments, a PEG may have an Mw of about 1000 Da, 5000 Da, 10,000 Da or 20,000 Da. In some embodiments, the PEG polymer may further be substituted with a hydrophilic or charged moiety, such as but not limited to an alcohol functionality or a carboxylic acid moiety.

The covalently linked moiety may include one or more saccharides. The covalently linked saccharides may be mono-, di-, or polysaccharides. The covalently linked saccharides may be modified to introduce a reactive pairing moiety which permits coupling or elaboration for attachment to the surface. One exemplary covalently linked moiety may include a dextran polysaccharide, which may be coupled indirectly to a surface via an unbranched linker.

The coating material providing a conditioned surface may comprise only one kind of covalently linked moiety or may include more than one different kind of covalently linked moiety. For example, a polyethylene glycol conditioned surface may have covalently linked alkylene oxide moieties having a specified number of alkylene oxide units which are all the same, e.g., having the same linking group and covalent attachment to the surface, the same overall length, and the same number of alkylene oxide units. Alternatively, the coating material may have more than one kind of covalently linked moiety attached to the surface. For example, the coating material may include the molecules having covalently linked alkylene oxide moieties having a first specified number of alkylene oxide units and may further include a further set of molecules having bulky moieties such as a protein or peptide connected to a covalently attached alkylene oxide linking moiety having a greater number of alkylene oxide units. The different types of molecules may be varied in any suitable ratio to obtain the surface characteristics desired. For example, the conditioned surface having a mixture of first molecules having a chemical structure having a first specified number of alkylene oxide units and second molecules including peptide or protein moieties, which may be coupled via a biotin/streptavidin binding pair to the covalently attached alkylene linking moiety, may have a ratio of first molecules:second molecules of about 99:1; about 90:10; about 75:25; about 50:50; about 30:70; about 20:80; about 10:90; or any ratio selected to be between these values. In this instance, the first set of molecules having different, less sterically demanding termini and fewer backbone atoms can help to functionalize the entire substrate surface and thereby prevent undesired adhesion or contact with the silicon/silicon oxide, hafnium oxide or alumina making up the substrate itself. The selection of the ratio of mixture of first molecules to second molecules may also modulate the surface modification introduced by the second molecules bearing peptide or protein moieties.

Conditioned surface properties. Various factors can alter the physical thickness of the conditioned surface, such as the manner in which the conditioned surface is formed on the substrate (e.g., vapor deposition, liquid phase deposition, spin coating, flooding, and electrostatic coating). In some embodiments, the conditioned surface may have a thickness of about 1 nm to about 10 nm. In some embodiments, the covalently linked moieties of the conditioned surface may form a monolayer when covalently linked to the surface of the microfluidic device (which may include an electrode activation substrate having dielectrophoresis (DEP) or electrowetting (EW) electrodes) and may have a thickness of less than 10 nm (e.g., less than 5 nm, or about 1.5 to 3.0 nm). These values are in contrast to that of a surface prepared by spin coating, for example, which may typically have a thickness of about 30 nm. In some embodiments, the conditioned surface does not require a perfectly formed monolayer to be suitably functional for operation within a DEP-configured microfluidic device. In other embodiments, the conditioned surface formed by the covalently linked moieties may have a thickness of about 10 nm to about 50 nm.

Unitary or Multi-part conditioned surface. The covalently linked coating material may be formed by reaction of a molecule which already contains the moiety configured to provide a layer of organic and/or hydrophilic molecules suitable for maintenance/expansion of biological micro-object(s) in the microfluidic device, and may have a structure of Formula I, as shown below. Alternatively, the covalently linked coating material may be formed in a two-part sequence, having a structure of Formula II, by coupling the moiety configured to provide a layer of organic and/or hydrophilic molecules suitable for maintenance and/or expansion of biological micro-object(s) to a surface modifying ligand that itself has been covalently linked to the surface. In some embodiments, the surface may be formed in a two-part or three-part sequence, including a streptavidin/biotin binding pair, to introduce a protein, peptide, or mixed modified surface.

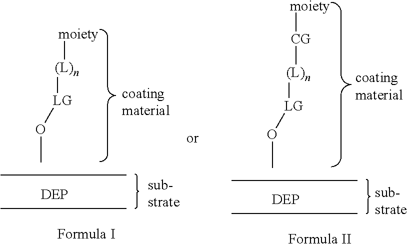

The coating material may be linked covalently to oxides of the surface of a DEP-configured or EW-configured substrate. The coating material may be attached to the oxides via a linking group (“LG”), which may be a siloxy or phosphonate ester group formed from the reaction of a siloxane or phosphonic acid group with the oxides. The moiety configured to provide a layer of organic and/or hydrophilic molecules suitable for maintenance/expansion of biological micro-object(s) in the microfluidic device can be any of the moieties described herein. The linking group LG may be directly or indirectly connected to the moiety configured to provide a layer of organic and/or hydrophilic molecules suitable for maintenance/expansion of biological micro-object(s) in the microfluidic device. When the linking group LG is directly connected to the moiety, optional linker (“L”) is not present and n is 0. When the linking group LG is indirectly connected to the moiety, linker L is present and n is 1. The linker L may have a linear portion where a backbone of the linear portion may include 1 to 200 non-hydrogen atoms selected from any combination of silicon, carbon, nitrogen, oxygen, sulfur and/or phosphorus atoms, subject to chemical bonding limitations as is known in the art. It may be interrupted with any combination of one or more moieties, which may be chosen from ether, amino, carbonyl, amido, and/or phosphonate groups, arylene, heteroarylene, or heterocyclic groups. In some embodiments, the coupling group CG represents the resultant group from reaction of a reactive moiety Rₓ and a reactive pairing moiety Rₚₓ (i.e., a moiety configured to react with the reactive moiety Rₓ). CG may be a carboxamidyl group, a triazolylene group, substituted triazolylene group, a carboxamidyl, thioamidyl, an oxime, a mercaptyl, a disulfide, an ether, or alkenyl group, or any other suitable group that may be formed upon reaction of a reactive moiety with its respective reactive pairing moiety. In some embodiments, CG may further represent a streptavidin/biotin binding pair.

Further details of suitable coating treatments and modifications, as well as methods of preparation, may be found at U.S. Patent Application Publication No. US2016/0312165 (Lowe, Jr., et al.), U.S. Patent Application Publication No US2017/0173580 (Lowe, Jr., et al), International Patent Application Publication WO2017/205830 (Lowe, Jr., et al.), and International Patent Application Publication WO2019/01880 (Beemiller et al.), each of which disclosures is herein incorporated by reference in its entirety.

Microfluidic device motive technologies. The microfluidic devices described herein can be used with any type of motive technology. As described herein, the control and monitoring equipment of the system can comprise a motive module for selecting and moving objects, such as micro-objects or droplets, in the microfluidic circuit of a microfluidic device. The motive technology (ies) may include, for example, dielectrophoresis (DEP), electrowetting (EW), and/or other motive technologies. The microfluidic device can have a variety of motive configurations, depending upon the type of object being moved and other considerations. Returning to FIG. 1A, for example, the support structure 104 and/or cover 110 of the microfluidic device 100 can comprise DEP electrode activation substrates for selectively inducing motive forces on micro-objects in the fluidic medium 180 in the microfluidic circuit 120 and thereby select, capture, and/or move individual micro-objects or groups of micro-objects.

In some embodiments, motive forces are applied across the fluidic medium 180 (e.g., in the flow path and/or in the sequestration pens) via one or more electrodes (not shown) to manipulate, transport, separate and sort micro-objects located therein. For example, in some embodiments, motive forces are applied to one or more portions of microfluidic circuit 120 in order to transfer a single micro-object from the flow path 106 into a desired microfluidic sequestration pen. In some embodiments, motive forces are used to prevent a micro-object within a sequestration pen from being displaced therefrom. Further, in some embodiments, motive forces are used to selectively remove a micro-object from a sequestration pen that was previously collected in accordance with the embodiments of the current disclosure.

In some embodiments, the microfluidic device is configured as an optically-actuated electrokinetic device, such as in optoelectronic tweezer (OET) and/or optoelectrowetting (OEW) configured device. Examples of suitable OET configured devices (e.g., containing optically actuated dielectrophoresis electrode activation substrates) can include those illustrated in U.S. Patent No. RE 44,711 (Wu, et al.) (originally issued as U.S. Pat. No. 7,612,355), U.S. Pat. No. 7,956,339 (Ohta, et al.), U.S. Pat. No. 9,908,115 (Hobbs et al.), and U.S. Pat. No. 9,403,172 (Short et al), each of which is incorporated herein by reference in its entirety. Examples of suitable OEW configured devices can include those illustrated in U.S. Pat. No. 6,958,132 (Chiou, et al.), and U.S. Pat. No. 9,533,306 (Chiou, et al.), each of which is incorporated herein by reference in its entirety. Examples of suitable optically-actuated electrokinetic devices that include combined OET/OEW configured devices can include those illustrated in U.S. Patent Application Publication No. 2015/0306598 (Khandros, et al.), U.S. Patent Application Publication No 2015/0306599 (Khandros, et al.), and U.S. Patent Application Publication No. 2017/0173580 (Lowe, et al.), each of which is incorporated herein by reference in its entirety.

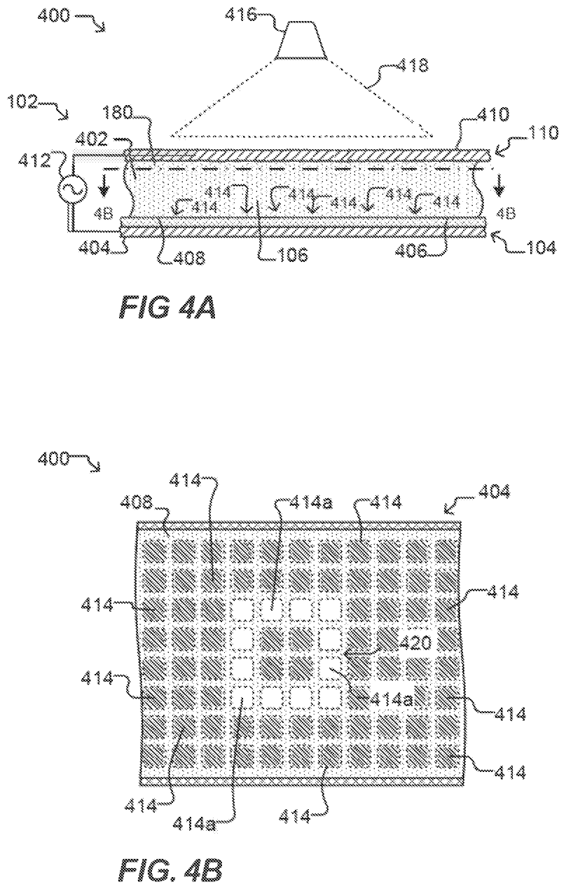

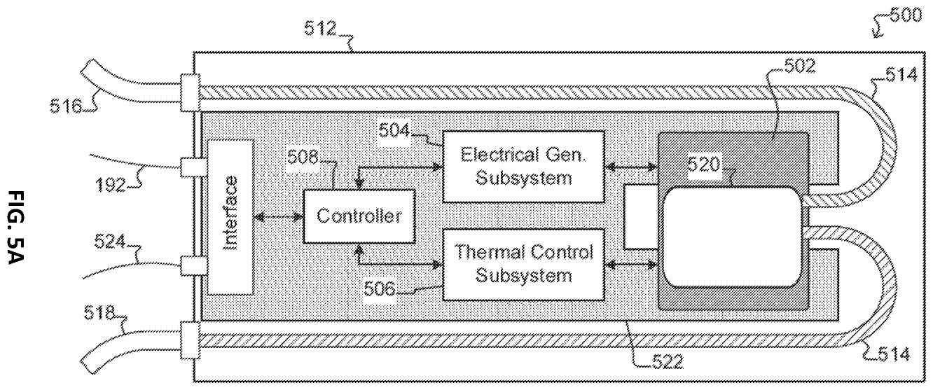

It should be understood that, for purposes of simplicity, the various examples of FIGS. 1-5B may illustrate portions of microfluidic devices while not depicting other portions. Further, FIGS. 1-5B may be part of, and implemented as, one or more microfluidic systems. In one non-limiting example, FIGS. 4A and 4B show a side cross-sectional view and a top cross-sectional view, respectively, of a portion of an enclosure 102 of the microfluidic device 400 having a region/chamber 402, which may be part of a fluidic circuit element having a more detailed structure, such as a growth chamber, a sequestration pen (which may be like any sequestration pen described herein), a flow region, or a flow channel. For instance, microfluidic device 400 may be similar to microfluidic devices 100, 175, 200, 300, 520 or any other microfluidic device as described herein. Furthermore, the microfluidic device 400 may include other fluidic circuit elements and may be part of a system including control and monitoring equipment 152, described above, having one or more of the media module 160, motive module 162, imaging module 164, optional tilting module 166, and other modules 168. Microfluidic devices 175, 200, 300, 520 and any other microfluidic devices described herein may similarly have any of the features described in detail for FIGS. 1A-1B and 4A-4B.

As shown in the example of FIG. 4A, the microfluidic device 400 includes a support structure 104 having a bottom electrode 404 and an electrode activation substrate 406 overlying the bottom electrode 404, and a cover 110 having a top electrode 410, with the top electrode 410 spaced apart from the bottom electrode 404. The top electrode 410 and the electrode activation substrate 406 define opposing surfaces of the region/chamber 402. A fluidic medium 180 contained in the region/chamber 402 thus provides a resistive connection between the top electrode 410 and the electrode activation substrate 406. A power source 412 configured to be connected to the bottom electrode 404 and the top electrode 410 and create a biasing voltage between the electrodes, as required for the generation of DEP forces in the region/chamber 402, is also shown. The power source 412 can be, for example, an alternating current (AC) power source.

In certain embodiments, the microfluidic device 400 illustrated in FIGS. 4A and 4B can have an optically-actuated DEP electrode activation substrate. Accordingly, changing patterns of light 418 from the light source 416, which may be controlled by the motive module 162, can selectively activate and deactivate changing patterns of DEP electrodes at regions 414 of the inner surface 408 of the electrode activation substrate 406. (Hereinafter the regions 414 of a microfluidic device having a DEP electrode activation substrate are referred to as “DEP electrode regions.”) As illustrated in FIG. 4B, a light pattern 418 directed onto the inner surface 408 of the electrode activation substrate 406 can illuminate select DEP electrode regions 414a (shown in white) in a pattern, such as a square. The non-illuminated DEP electrode regions 414 (cross-hatched) are hereinafter referred to as “dark” DEP electrode regions 414. The relative electrical impedance through the DEP electrode activation substrate 406 (i.e., from the bottom electrode 404 up to the inner surface 408 of the electrode activation substrate 406 which interfaces with the fluidic medium 180 in the flow region 106) is greater than the relative electrical impedance through the fluidic medium 180 in the region/chamber 402 (i.e., from the inner surface 408 of the electrode activation substrate 406 to the top electrode 410 of the cover 110) at each dark DEP electrode region 414. An illuminated DEP electrode region 414a, however, exhibits a reduced relative impedance through the electrode activation substrate 406 that is less than the relative impedance through the fluidic medium 180 in the region/chamber 402 at each illuminated DEP electrode region 414a.

With the power source 412 activated, the foregoing DEP configuration creates an electric field gradient in the fluidic medium 180 between illuminated DEP electrode regions 414a and adjacent dark DEP electrode regions 414, which in turn creates local DEP forces that attract or repel nearby micro-objects (not shown) in the fluidic medium 180. DEP electrodes that attract or repel micro-objects in the fluidic medium 180 can thus be selectively activated and deactivated at many different such DEP electrode regions 414 at the inner surface 408 of the region/chamber 402 by changing light patterns 418 projected from a light source 416 into the microfluidic device 400. Whether the DEP forces attract or repel nearby micro-objects can depend on such parameters as the frequency of the power source 412 and the dielectric properties of the fluidic medium 180 and/or micro-objects (not shown). Depending on the frequency of the power applied to the DEP configuration and selection of fluidic media (e.g., a highly conductive media such as PBS or other media appropriate for maintaining biological cells), negative DEP forces may be produced. Negative DEP forces may repel the micro-objects away from the location of the induced non-uniform electrical field. In some embodiments, a microfluidic device incorporating DEP technology may generate negative DEP forces.

The square pattern 420 of illuminated DEP electrode regions 414a illustrated in FIG. 4B is an example only. Any pattern of the DEP electrode regions 414 can be illuminated (and thereby activated) by the pattern of light 418 projected into the microfluidic device 400, and the pattern of illuminated/activated DEP electrode regions 414 can be repeatedly changed by changing or moving the light pattern 418.

In some embodiments, the electrode activation substrate 406 can comprise or consist of a photoconductive material. In such embodiments, the inner surface 408 of the electrode activation substrate 406 can be featureless. For example, the electrode activation substrate 406 can comprise or consist of a layer of hydrogenated amorphous silicon (a-Si:H). The a-Si:H can comprise, for example, about 8% to 40% hydrogen (calculated as 100*the number of hydrogen atoms/the total number of hydrogen and silicon atoms). The layer of a-Si:H can have a thickness of about 500 nm to about 2.0 μm. In such embodiments, the DEP electrode regions 414 can be created anywhere and in any pattern on the inner surface 408 of the electrode activation substrate 406, in accordance with the light pattern 418. The number and pattern of the DEP electrode regions 414 thus need not be fixed, but can correspond to the light pattern 418. Examples of microfluidic devices having a DEP configuration comprising a photoconductive layer such as discussed above have been described, for example, in U.S. Patent No. RE 44,711 (Wu, et al.) (originally issued as U.S. Pat. No. 7,612,355), each of which is incorporated herein by reference in its entirety.

In other embodiments, the electrode activation substrate 406 can comprise a substrate comprising a plurality of doped layers, electrically insulating layers (or regions), and electrically conductive layers that form semiconductor integrated circuits, such as is known in semiconductor fields. For example, the electrode activation substrate 406 can comprise a plurality of phototransistors, including, for example, lateral bipolar phototransistors, with each phototransistor corresponding to a DEP electrode region 414. Alternatively, the electrode activation substrate 406 can comprise electrodes (e.g., conductive metal electrodes) controlled by phototransistor switches, with each such electrode corresponding to a DEP electrode region 414. The electrode activation substrate 406 can include a pattern of such phototransistors or phototransistor-controlled electrodes. The pattern, for example, can be an array of substantially square phototransistors or phototransistor-controlled electrodes arranged in rows and columns. Alternatively, the pattern can be an array of substantially hexagonal phototransistors or phototransistor-controlled electrodes that form a hexagonal lattice. Regardless of the pattern, electric circuit elements can form electrical connections between the DEP electrode regions 414 at the inner surface 408 of the electrode activation substrate 406 and the bottom electrode 404, and those electrical connections (i.e., phototransistors or electrodes) can be selectively activated and deactivated by the light pattern 418, as described above.

Examples of microfluidic devices having electrode activation substrates that comprise phototransistors have been described, for example, in U.S. Pat. No. 7,956,339 (Ohta et al.) and U.S. Pat. No. 9,908,115 (Hobbs et al.), the entire contents of each of which are incorporated herein by reference. Examples of microfluidic devices having electrode activation substrates that comprise electrodes controlled by phototransistor switches have been described, for example, in U.S. Pat. No. 9,403,172 (Short et al.), which is incorporated herein by reference in its entirety.

In some embodiments of a DEP configured microfluidic device, the top electrode 410 is part of a first wall (or cover 110) of the enclosure 402, and the electrode activation substrate 406 and bottom electrode 404 are part of a second wall (or support structure 104) of the enclosure 102. The region/chamber 402 can be between the first wall and the second wall. In other embodiments, the electrode 410 is part of the second wall (or support structure 104) and one or both of the electrode activation substrate 406 and/or the electrode 410 are part of the first wall (or cover 110). Moreover, the light source 416 can alternatively be used to illuminate the enclosure 102 from below.

With the microfluidic device 400 of FIGS. 4A-4B having a DEP electrode activation substrate, the motive module 162 of control and monitoring equipment 152, as described for FIG. 1A herein, can select a micro-object (not shown) in the fluidic medium 180 in the region/chamber 402 by projecting a light pattern 418 into the microfluidic device 400 to activate a first set of one or more DEP electrodes at DEP electrode regions 414a of the inner surface 408 of the electrode activation substrate 406 in a pattern (e.g., square pattern 420) that surrounds and captures the micro-object. The motive module 162 can then move the in situ-generated captured micro-object by moving the light pattern 418 relative to the microfluidic device 400 to activate a second set of one or more DEP electrodes at DEP electrode regions 414. Alternatively, the microfluidic device 400 can be moved relative to the light pattern 418.

In other embodiments, the microfluidic device 400 may be a DEP configured device that does not rely upon light activation of DEP electrodes at the inner surface 408 of the electrode activation substrate 406. For example, the electrode activation substrate 406 can comprise selectively addressable and energizable electrodes positioned opposite to a surface including at least one electrode (e.g., cover 110). Switches (e.g., transistor switches in a semiconductor substrate) may be selectively opened and closed to activate or inactivate DEP electrodes at DEP electrode regions 414, thereby creating a net DEP force on a micro-object (not shown) in region/chamber 402 in the vicinity of the activated DEP electrodes. Depending on such characteristics as the frequency of the power source 412 and the dielectric properties of the medium (not shown) and/or micro-objects in the region/chamber 402, the DEP force can attract or repel a nearby micro-object. By selectively activating and deactivating a set of DEP electrodes (e.g., at a set of DEP electrodes regions 414 that forms a square pattern 420), one or more micro-objects in region/chamber 402 can be selected and moved within the region/chamber 402. The motive module 162 in FIG. 1A can control such switches and thus activate and deactivate individual ones of the DEP electrodes to select, and move particular micro-objects (not shown) around the region/chamber 402. Microfluidic devices having a DEP electrode activation substrate that includes selectively addressable and energizable electrodes are known in the art and have been described, for example, in U.S. Pat. No. 6,294,063 (Becker, et al.) and U.S. Pat. No. 6,942,776 (Medoro), each of which is incorporated herein by reference in its entirety.

Regardless of whether the microfluidic device 400 has a dielectrophoretic electrode activation substrate, an electrowetting electrode activation substrate or a combination of both a dielectrophoretic and an electrowetting activation substrate, a power source 412 can be used to provide a potential (e.g., an AC voltage potential) that powers the electrical circuits of the microfluidic device 400. The power source 412 can be the same as, or a component of, the power source 192 referenced in FIG. 1A. Power source 412 can be configured to provide an AC voltage and/or current to the top electrode 410 and the bottom electrode 404. For an AC voltage, the power source 412 can provide a frequency range and an average or peak power (e.g., voltage or current) range sufficient to generate net DEP forces (or electrowetting forces) strong enough to select and move individual micro-objects (not shown) in the region/chamber 402, as discussed above, and/or to change the wetting properties of the inner surface 408 of the support structure 104 in the region/chamber 202, as also discussed above. Such frequency ranges and average or peak power ranges are known in the art. See, e.g., U.S. Pat. No. 6,958,132 (Chiou, et al.), U.S. Pat. No. RE44,711 (Wu, et al.) (originally issued as U.S. Pat. No. 7,612,355), and U.S. Patent Application Publication Nos. 2014/0124370 (Short, et al.), 2015/0306598 (Khandros, et al.), 2015/0306599 (Khandros, et al.), and 2017/0173580 (Lowe, Jr. et al.), each of which disclosures are herein incorporated by reference in its entirety.

Other forces may be utilized within the microfluidic devices, alone or in combination, to move selected micro-objects. Bulk fluidic flow within the microfluidic channel may move micro-objects within the flow region. Localized fluidic flow, which may be operated within the microfluidic channel, within a sequestration pen, or within another kind of chamber (e.g., a reservoir) can also be used to move selected micro-objects. Localized fluidic flow can be used to move selected micro-objects out of the flow region into a non-flow region such as a sequestration pen or the reverse, from a non-flow region into a flow region. The localized flow can be actuated by deforming a deformable wall of the microfluidic device, as described in U.S. Pat. No. 10,058,865 (Breinlinger, et al.), which is incorporated herein by reference in its entirety.

Gravity may be used to move micro-objects within the microfluidic channel, into a sequestration pen, and/or out of a sequestration pen or other chamber, as described in U.S. Pat. No. 9,744,533 (Breinlinger, et al.), which is incorporated herein by reference in its entirety. Use of gravity (e.g., by tilting the microfluidic device and/or the support to which the microfluidic device is attached) may be useful for bulk movement of cells into or out of the sequestration pens from/to the flow region. Magnetic forces may be employed to move micro-objects including paramagnetic materials, which can include magnetic micro-objects attached to or associated with a biological micro-object. Alternatively, or in additional, centripetal forces may be used to move micro-objects within the microfluidic channel, as well as into or out of sequestration pens or other chambers in the microfluidic device.

In another alternative mode of moving micro-objects, laser-generated dislodging forces may be used to export micro-objects or assist in exporting micro-objects from a sequestration pen or any other chamber in the microfluidic device, as described in U.S. Pat. No. 10,829,728 (Kurz, et al.), filed Jun. 15, 2018, and granted Nov. 10, 2020, which is incorporated herein by reference in its entirety.

In some embodiments, DEP forces are combined with other forces, such as fluidic flow (e.g., bulk fluidic flow in a channel or localized fluidic flow actuated by deformation of a deformable surface of the microfluidic device, laser generated dislodging forces, and/or gravitational force), so as to manipulate, transport, separate and sort micro-objects and/or droplets within the microfluidic circuit 120. In some embodiments, the DEP forces can be applied prior to the other forces. In other embodiments, the DEP forces can be applied after the other forces. In still other instances, the DEP forces can be applied in an alternating manner with the other forces. For the microfluidic devices described herein, repositioning of micro-objects may not generally rely upon gravity or hydrodynamic forces to position or trap micro-objects at a selected position. Gravity may be chosen as one form of repositioning force, but the ability to reposition of micro-objects within the microfluidic device does not rely solely upon the use of gravity. While fluid flow in the microfluidic channels may be used to introduce micro-objects into the microfluidic channels (e.g., flow region), such regional flow is not relied upon to pen or unpen micro-objects, while localized flow (e.g., force derived from actuating a deformable surface) may, in some embodiments, be selected from amongst the other types of repositioning forces described herein to pen or unpen micro-objects or to export them from the microfluidic device.

When DEP is used to reposition micro-objects, bulk fluidic flow in a channel is generally stopped prior to applying DEP to micro-objects to reposition the micro-objects within the microfluidic circuit of the device, whether the micro-objects are being repositioned from the channel into a sequestration pen or from a sequestration pen into the channel. Bulk fluidic flow may be resumed thereafter.

System. Returning to FIG. 1A, a system 150 for operating and controlling microfluidic devices is shown, such as for controlling the microfluidic device 100. System 150 control its various components (e.g., media module 160, motive module 162, imaging module 164, tilting module 166, and other modules 168) to implement any of the techniques described herein. For example, master controller 154 can include a control module 156 (e.g., a processor or controller circuit) configured to access a digital memory 158 storing computer instructions that, when executed by one or more computing devices (e.g., control module 156), the computer program instructions cause the one or more computing devices to implement the techniques described herein including forming the in situ-generated structures, control light sources to project upon a microfluidic device, and facilitate displace of micro-objects.

The electrical power source 192 can provide electric power to the microfluidic device 100, providing biasing voltages or currents as needed. The electrical power source 192 can, for example, comprise one or more alternating current (AC) and/or direct current (DC) voltage or current sources.

System 150 can further include a media source 178. The media source 178 (e.g., a container, reservoir, or the like) can comprise multiple sections or containers, each for holding a different fluidic medium 180. Thus, the media source 178 can be a device that is outside of and separate from the microfluidic device 100, as illustrated in FIG. 1A. Alternatively, the media source 178 can be located in whole or in part inside the enclosure 102 of the microfluidic device 100. For example, the media source 178 can comprise reservoirs that are part of the microfluidic device 100.

FIG. 1A also illustrates simplified block diagram depictions of examples of control and monitoring equipment 152 that constitute part of system 150 and can be utilized in conjunction with a microfluidic device 100. As shown, examples of such control and monitoring equipment 152 can include a master controller 154 comprising a media module 160 for controlling the media source 178, a motive module 162 for controlling movement and/or selection of micro-objects (not shown) and/or medium (e.g., droplets of medium) in the microfluidic circuit 120, an imaging module 164 for controlling an imaging device (e.g., a camera, microscope, light source or any combination thereof) for capturing images (e.g., digital images), and an optional tilting module 166 for controlling the tilting of the microfluidic device 100. The control equipment 152 can also include other modules 168 for controlling, monitoring, or performing other functions with respect to the microfluidic device 100. As shown, the monitoring equipment 152 can further include a display device 170 and an input/output device 172.

The master controller 154 can comprise a control module 156 and a digital memory 158. The control module 156 can comprise, for example, a digital processor configured to operate in accordance with machine executable instructions (e.g., software, firmware, source code, or the like) stored as non-transitory data or signals in the memory 158. Alternatively, or in addition, the control module 156 can comprise hardwired digital circuitry and/or analog circuitry. The media module 160, motive module 162, imaging module 164, optional tilting module 166, and/or other modules 168 can be similarly configured. Thus, functions, processes acts, actions, or steps of a process discussed herein as being performed with respect to the microfluidic device 100 or any other microfluidic apparatus can be performed by any one or more of the master controller 154, media module 160, motive module 162, imaging module 164, optional tilting module 166, and/or other modules 168 configured as discussed above. Similarly, the master controller 154, media module 160, motive module 162, imaging module 164, optional tilting module 166, and/or other modules 168 may be communicatively coupled to transmit and receive data used in any function, process, act, action or step discussed herein.

The media module 160 controls the media source 178. For example, the media module 160 can control the media source 178 to input a selected fluidic medium 180 into the enclosure 102 (e.g., through an inlet port 107). The media module 160 can also control removal of media from the enclosure 102 (e.g., through an outlet port (not shown)). One or more media can thus be selectively input into and removed from the microfluidic circuit 120. The media module 160 can also control the flow of fluidic medium 180 in the flow path 106 inside the microfluidic circuit 120. The media module 160 may also provide conditioning gaseous conditions to the media source 178, for example, providing an environment containing 5% CO₂ (or higher). The media module 160 may also control the temperature of an enclosure of the media source, for example, to provide feeder cells in the media source with proper temperature control.

Motive module. The motive module 162 can be configured to control selection and movement of micro-objects (not shown) in the microfluidic circuit 120. The enclosure 102 of the microfluidic device 100 can comprise one or more electrokinetic mechanisms including a dielectrophoresis (DEP) electrode activation substrate, optoelectronic tweezers (OET) electrode activation substrate, electrowetting (EW) electrode activation substrate, and/or an opto-electrowetting (OEW) electrode activation substrate, where the motive module 162 can control the activation of electrodes and/or transistors (e.g., phototransistors) to select and move micro-objects and/or droplets in the flow path 106 and/or within sequestration pens 124, 126, 128, and 130. The electrokinetic mechanism(s) may be any suitable single or combined mechanism as described within the paragraphs describing motive technologies for use within the microfluidic device. A DEP configured device may include one or more electrodes that apply a non-uniform electric field in the microfluidic circuit 120 sufficient to exert a dielectrophoretic force on micro-objects in the microfluidic circuit 120. An OET configured device may include photo-activatable electrodes to provide selective control of movement of micro-objects in the microfluidic circuit 120 via light-induced dielectrophoresis.

The imaging module 164 can control the imaging device. For example, the imaging module 164 can receive and process image data from the imaging device. Image data from the imaging device can comprise any type of information captured by the imaging device (e.g., the presence or absence of micro-objects, droplets of medium, accumulation of label, such as fluorescent label, etc.). Using the information captured by the imaging device, the imaging module 164 can further calculate the position of objects (e.g., micro-objects, droplets of medium) and/or the rate of motion of such objects within the microfluidic device 100.

The imaging device (part of imaging module 164, discussed below) can comprise a device, such as a digital camera, for capturing images inside microfluidic circuit 120. In some instances, the imaging device further comprises a detector having a fast frame rate and/or high sensitivity (e.g., for low light applications). The imaging device can also include a mechanism for directing stimulating radiation and/or light beams into the microfluidic circuit 120 and collecting radiation and/or light beams reflected or emitted from the microfluidic circuit 120 (or micro-objects contained therein). The emitted light beams may be in the visible spectrum and may, e.g., include fluorescent emissions. The reflected light beams may include reflected emissions originating from an LED or a wide spectrum lamp, such as a mercury lamp (e.g., a high-pressure mercury lamp) or a Xenon arc lamp. The imaging device may further include a microscope (or an optical train), which may or may not include an eyepiece.

Support Structure. System 150 may further comprise a support structure 190 configured to support and/or hold the enclosure 102 comprising the microfluidic circuit 120. In some embodiments, the optional tilting module 166 can be configured to activate the support structure 190 to rotate the microfluidic device 100 about one or more axes of rotation. The optional tilting module 166 can be configured to support and/or hold the microfluidic device 100 in a level orientation (i.e., at 0° relative to x- and y-axes), a vertical orientation (i.e., at 90° relative to the x-axis and/or the y-axis), or any orientation therebetween. The orientation of the microfluidic device 100 (and the microfluidic circuit 120) relative to an axis is referred to herein as the “tilt” of the microfluidic device 100 (and the microfluidic circuit 120). For example, support structure 190 can optionally be used to tilt the microfluidic device 100 (e.g., as controlled by optional tilting module 166) to 0.1°, 0.2°, 0.3°, 0.4°, 0.5°, 0.6°, 0.7°, 0.8°, 0.9°, 1°, 2°, 3°, 4°, 5°, 10°, 15°, 20°, 25°, 30°, 35°, 40°, 45°, 50°, 55°, 60°, 65°, 70°, 75°, 80°, 90° relative to the x-axis or any degree therebetween. When the microfluidic device is tilted at angles greater than about 15, tilting may be performed to create bulk movement of micro-objects into/out of sequestration pens from/into the flow region (e.g., microfluidic channel). In some embodiments, the support structure 190 can hold the microfluidic device 100 at a fixed angle of 0.1°, 0.2°, 0.3°, 0.4°, 0.5°, 0.6°, 0.7°, 0.8°, 0.9°, 1°, 2°, 3°, 4°, 5°, or 10° relative to the x-axis (horizontal), so long as DEP is an effective force to move micro-objects out of the sequestration pens into the microfluidic channel. Since the surface of the electrode activation substrate is substantially flat, DEP forces may be used even when the far end of the sequestration pen, opposite its opening to the microfluidic channel, is disposed at a position lower in a vertical direction than the microfluidic channel.

In some embodiments where the microfluidic device is tilted or held at a fixed angle relative to horizontal, the microfluidic device 100 may be disposed in an orientation such that the inner surface of the base of the flow path 106 is positioned at an angle above or below the inner surface of the base of the one or more sequestration pens opening laterally to the flow path. The term “above” as used herein denotes that the flow path 106 is positioned higher than the one or more sequestration pens on a vertical axis defined by the force of gravity (i.e., an object in a sequestration pen above a flow path 106 would have a higher gravitational potential energy than an object in the flow path), and inversely, for positioning of the flow path 106 below one or more sequestration pens. In some embodiments, the support structure 190 may be held at a fixed angle of less than about 5°, about 4°, about 3° or less than about 2° relative to the x-axis (horizontal), thereby placing the sequestration pens at a lower potential energy relative to the flow path. In some other embodiments, when long term culturing (e.g., for more than about 2, 3, 4, 5, 6, 7 or more days) is performed within the microfluidic device, the device may be supported on a culturing support and may be tilted at a greater angle of about 10°, 15°, 20°, 25°, 30°, or any angle therebetween to retain biological micro-objects within the sequestration pens during the long-term culturing period. At the end of the culturing period, the microfluidic device containing the cultured biological micro-objects may be returned to the support 190 within system 150, where the angle of tilting is decreased to values as described above, affording the use of DEP to move the biological micro-objects out of the sequestration pens. Further examples of the use of gravitational forces induced by tilting are described in U.S. Pat. No. 9,744,533 (Breinlinger et al.), the contents of which are herein incorporated by reference in its entirety.

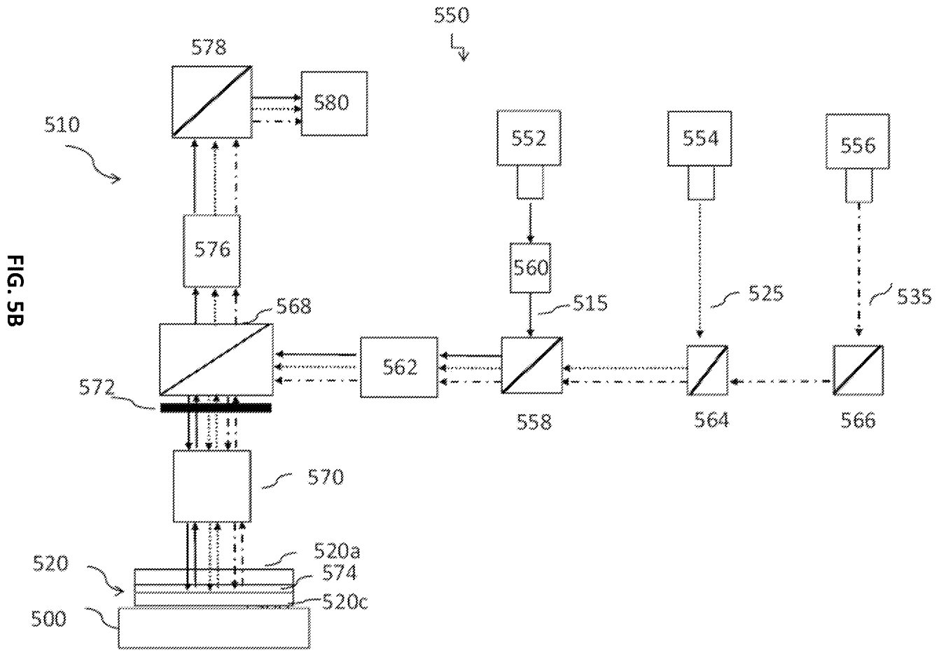

Nest. Turning now to FIG. 5A, the system 150 can include a structure (also referred to as a “nest”) 500 configured to hold a microfluidic device 520, which may be like microfluidic device 100, 200, or any other microfluidic device described herein. The nest 500 can include a socket 502 capable of interfacing with the microfluidic device 520 (e.g., an optically actuated electrokinetic device 100, 200, etc.) and providing electrical connections from power source 192 to microfluidic device 520. The nest 500 can further include an integrated electrical signal generation subsystem 504. The electrical signal generation subsystem 504 can be configured to supply a biasing voltage to socket 502 such that the biasing voltage is applied across a pair of electrodes in the microfluidic device 520 when it is being held by socket 502. Thus, the electrical signal generation subsystem 504 can be part of power source 192. The ability to apply a biasing voltage to microfluidic device 520 does not mean that a biasing voltage will be applied at all times when the microfluidic device 520 is held by the socket 502. Rather, in most cases, the biasing voltage will be applied intermittently, e.g., only as needed to facilitate the generation of electrokinetic forces, such as dielectrophoresis or electro-wetting, in the microfluidic device 520.

As illustrated in FIG. 5A, the nest 500 can include a printed circuit board assembly (PCBA) 522. The electrical signal generation subsystem 504 can be mounted on and electrically integrated into the PCBA 522. The exemplary support includes socket 502 mounted on PCBA 522, as well.

In some embodiments, the nest 500 can comprise an electrical signal generation subsystem 504 configured to measure the amplified voltage at the microfluidic device 520 and then adjust its own output voltage as needed such that the measured voltage at the microfluidic device 520 is the desired value. In some embodiments, the waveform amplification circuit can have a +6.5V to −6.5V power supply generated by a pair of DC-DC converters mounted on the PCBA 522, resulting in a signal of up to 13 Vpp at the microfluidic device 520.

In certain embodiments, the nest 500 further comprises a controller 508, such as a microprocessor used to sense and/or control the electrical signal generation subsystem 504. Examples of suitable microprocessors include the Arduino™ microprocessors, such as the Arduino Nano™. The controller 508 may be used to perform functions and analysis or may communicate with an external master controller 154 (shown in FIG. 1A) to perform functions and analysis. In the embodiment illustrated in FIG. 5A the controller 508 communicates with the master controller 154 (of FIG. 1A) through an interface (e.g., a plug or connector).

As illustrated in FIG. 5A, the support structure 500 (e.g., nest) can further include a thermal control subsystem 506. The thermal control subsystem 506 can be configured to regulate the temperature of microfluidic device 520 held by the support structure 500. For example, the thermal control subsystem 506 can include a Peltier thermoelectric device (not shown) and a cooling unit (not shown). In the embodiment illustrated in FIG. 5A, the support structure 500 comprises an inlet 516 and an outlet 518 to receive cooled fluid from an external reservoir (not shown) of the cooling unit, introduce the cooled fluid into the fluidic path 514 and through the cooling block, and then return the cooled fluid to the external reservoir. In some embodiments, the Peltier thermoelectric device, the cooling unit, and/or the fluidic path 514 can be mounted on a casing 512 of the support structure 500. In some embodiments, the thermal control subsystem 506 is configured to regulate the temperature of the Peltier thermoelectric device so as to achieve a target temperature for the microfluidic device 520. Temperature regulation of the Peltier thermoelectric device can be achieved, for example, by a thermoelectric power supply, such as a Pololu™ thermoelectric power supply (Pololu Robotics and Electronics Corp.). The thermal control subsystem 506 can include a feedback circuit, such as a temperature value provided by an analog circuit. Alternatively, the feedback circuit can be provided by a digital circuit.

The nest 500 can include a serial port 524 which allows the microprocessor of the controller 508 to communicate with an external master controller 154 via the interface. In addition, the microprocessor of the controller 508 can communicate (e.g., via a Plink tool (not shown)) with the electrical signal generation subsystem 504 and thermal control subsystem 506. Thus, via the combination of the controller 508, the interface, and the serial port 524, the electrical signal generation subsystem 504 and the thermal control subsystem 506 can communicate with the external master controller 154. In this manner, the master controller 154 can, among other things, assist the electrical signal generation subsystem 504 by performing scaling calculations for output voltage adjustments. A Graphical User Interface (GUI) (not shown) provided via a display device 170 coupled to the external master controller 154, can be configured to plot temperature and waveform data obtained from the thermal control subsystem 506 and the electrical signal generation subsystem 504, respectively. Alternatively, or in addition, the GUI can allow for updates to the controller 508, the thermal control subsystem 506, and the electrical signal generation subsystem 504.

Optical sub-system. FIG. 5B is a schematic of an optical sub-system 550 having an optical apparatus 510 for imaging and manipulating micro-objects in a microfluidic device 520, which can be any microfluidic device described herein. The optical apparatus 510 can be configured to perform imaging, analysis and manipulation of one or more micro-objects within the enclosure of the microfluidic device 520.

The optical apparatus 510 may have a first light source 552, a second light source 554, and a third light source 556. The first light source 552 can transmit light to a structured light modulator 560, which can include a digital mirror device (DMD) or a microshutter array system (MSA), either of which can be configured to receive light from the first light source 552 and selectively transmit a subset of the received light into the optical apparatus 510. Alternatively, the structured light modulator 560 can include a device that produces its own light (and thus dispenses with the need for a light source 552), such as an organic light emitting diode display (OLED), a liquid crystal on silicon (LCOS) device, a ferroelectric liquid crystal on silicon device (FLCOS), or a transmissive liquid crystal display (LCD). The structured light modulator 560 can be, for example, a projector. Thus, the structured light modulator 560 can be capable of emitting both structured and unstructured light. In certain embodiments, an imaging module and/or motive module of the system can control the structured light modulator 560.

In embodiments when the structured light modulator 560 includes a mirror, the modulator can have a plurality of mirrors. Each mirror of the plurality of mirrors can have a size of about 5 microns×5 microns to about 10 microns x10 microns, or any values therebetween. The structured light modulator 560 can include an array of mirrors (or pixels) that is 2000×1000, 2580×1600, 3000×2000, or any values therebetween. In some embodiments, only a portion of an illumination area of the structured light modulator 560 is used. The structured light modulator 560 can transmit the selected subset of light to a first dichroic beam splitter 558, which can reflect this light to a first tube lens 562.

The first tube lens 562 can have a large clear aperture, for example, a diameter larger than about 40 mm to about 50 mm, or more, providing a large field of view. Thus, the first tube lens 562 can have an aperture that is large enough to capture all (or substantially all) of the light beams emanating from the structured light modulator 560.

The structured light 515 having a wavelength of about 400 nm to about 710 nm, may alternatively or in addition, provide fluorescent excitation illumination to the microfluidic device.

The second light source 554 may provide unstructured brightfield illumination. The brightfield illumination light 525 may have any suitable wavelength, and in some embodiments, may have a wavelength of about 400 nm to about 760 nm. The second light source 554 can transmit light to a second dichroic beam splitter 564 (which also may receive illumination light 535 from the third light source 556), and the second light, brightfield illumination light 525, may be transmitted therefrom to the first dichroic beam splitter 558. The second light, brightfield illumination light 525, may then be transmitted from the first dichroic beam splitter 558 to the first tube lens 562.

The third light source 556 can transmit light through a matched pair relay lens (not shown) to a mirror 566. The third illumination light 535 may therefrom be reflected to the second dichroic beam splitter 564 and be transmitted therefrom to the first beam splitter 558, and onward to the first tube lens 562. The third illumination light 535 may be a laser and may have any suitable wavelength. In some embodiments, the laser illumination 535 may have a wavelength of about 350 nm to about 900 nm. The laser illumination 535 may be configured to heat portions of one or more sequestration pens within the microfluidic device. The laser illumination 535 may be configured to heat fluidic medium, a micro-object, a wall or a portion of a wall of a sequestration pen, a metal target disposed within a microfluidic channel or sequestration pen of the microfluidic channel, or a photoreversible physical barrier within the microfluidic device, and described in more detail in U. S. Application Publication Nos. 2017/0165667 (Beaumont, et al.) and 2018/0298318 (Kurz, et al.), each of which disclosure is herein incorporated by reference in its entirety. In other embodiments, the laser illumination 535 may be configured to initiate photocleavage of surface modifying moieties of a modified surface of the microfluidic device or photocleavage of moieties providing adherent functionalities for micro-objects within a sequestration pen within the microfluidic device. Further details of photocleavage using a laser may be found in International Application Publication No. WO2017/205830 (Lowe, Jr. et al.), which disclosure is herein incorporated by reference in its entirety.

The light from the first, second, and third light sources (552, 554, 556) passes through the first tube lens 562 and is transmitted to a third dichroic beam splitter 568 and filter changer 572. The third dichroic beam splitter 568 can reflect a portion of the light and transmit the light through one or more filters in the filter changer 572 and to the objective 570, which may be an objective changer with a plurality of different objectives that can be switched on demand. Some of the light (515, 525, and/or 535) may pass through the third dichroic beam splitter 568 and be terminated or absorbed by a beam block (not shown). The light reflected from the third dichroic beam splitter 568 passes through the objective 570 to illuminate the sample plane 574, which can be a portion of a microfluidic device 520 such as the sequestration pens described herein.

The nest 500, as described in FIG. 5A, can be integrated with the optical apparatus 510 and be a part of the apparatus 510. The nest 500 can provide electrical connection to the enclosure and be further configured to provide fluidic connections to the enclosure. Users may load the microfluidic apparatus 520 into the nest 500. In some other embodiments, the nest 500 can be a separate component independent of the optical apparatus 510.

Light can be reflected off and/or emitted from the sample plane 574 to pass back through the objective 570, through the filter changer 572, and through the third dichroic beam splitter 568 to a second tube lens 576. The light can pass through the second tube lens 576 (or imaging tube lens 576) and be reflected from a mirror 578 to an imaging sensor 580. Stray light baffles (not shown) can be placed between the first tube lens 562 and the third dichroic beam splitter 568, between the third dichroic beam splitter 568 and the second tube lens 576, and between the second tube lens 576 and the imaging sensor 580.

Objective. The optical apparatus can comprise the objective lens 570 that is specifically designed and configured for viewing and manipulating of micro-objects in the microfluidic device 520. For example, conventional microscope objective lenses are designed to view micro-objects on a slide or through 5 mm of aqueous fluid, while micro-objects in the microfluidic device 520 are inside the plurality of sequestration pens within the viewing plane 574 which have a depth of 20, 30, 40, 50, 60 70, 80 microns or any values therebetween. In some embodiments, a transparent cover 520a, for example, glass or ITO cover with a thickness of about 750 microns, can be placed on top of the plurality of sequestration pens, which are disposed above a microfluidic substrate 520c. Thus, the images of the micro-objects obtained by using the conventional microscope objective lenses may have large aberrations such as spherical and chromatic aberrations, which can degrade the quality of the images. The objective lens 570 of the optical apparatus 510 can be configured to correct the spherical and chromatic aberrations in the optical apparatus 510. The objective lens 570 can have one or more magnification levels available such as, 4X, 10X, 20X.

Modes of illumination. In some embodiments, the structured light modulator 560 can be configured to modulate light beams received from the first light source 552 and transmits a plurality of illumination light beams 515, which are structured light beams, into the enclosure of the microfluidic device, e.g., the region containing the sequestration pens. The structured light beams can comprise the plurality of illumination light beams. The plurality of illumination light beams can be selectively activated to generate a plurality of illuminations patterns. In some embodiments, the structured light modulator 560 can be configured to generate an illumination pattern, similarly as described for FIGS. 4A-4B, which can be moved and adjusted. The optical apparatus 510 can further comprise a control unit (not shown) which is configured to adjust the illumination pattern to selectively activate the one or more of the plurality of DEP electrodes of a substrate 520c and generate DEP forces to move the one or more micro-objects inside the plurality of sequestration pens within the microfluidic device 520. For example, the plurality of illuminations patterns can be adjusted over time in a controlled manner to manipulate the micro-objects in the microfluidic device 520. Each of the plurality of illumination patterns can be shifted to shift the location of the DEP force generated and to move the structured light for one position to another in order to move the micro-objects within the enclosure of the microfluidic apparatus 520.

In some embodiments, the optical apparatus 510 may be configured such that each of the plurality of sequestration pens in the sample plane 574 within the field of view is simultaneously in focus at the image sensor 580 and at the structured light modulator 560. In some embodiments, the structured light modulator 560 can be disposed at a conjugate plane of the image sensor 580. In various embodiments, the optical apparatus 510 can have a confocal configuration or confocal property. The optical apparatus 510 can be further configured such that only each interior area of the flow region and/or each of the plurality of sequestration pens in the sample plane 574 within the field of view is imaged onto the image sensor 580 in order to reduce overall noise to thereby increase the contrast and resolution of the image.

In some embodiments, the first tube lens 562 can be configured to generate collimated light beams and transmit the collimated light beams to the objective lens 570. The objective 570 can receive the collimated light beams from the first tube lens 562 and focus the collimated light beams into each interior area of the flow region and each of the plurality of sequestration pens in the sample plane 574 within the field of view of the image sensor 580 or the optical apparatus 510. In some embodiments, the first tube lens 562 can be configured to generate a plurality of collimated light beams and transmit the plurality of collimated light beams to the objective lens 570. The objective 570 can receive the plurality of collimated light beams from the first tube lens 562 and converge the plurality of collimated light beams into each of the plurality of sequestration pens in the sample plane 574 within the field of view of the image sensor 580 or the optical apparatus 510.

In some embodiments, the optical apparatus 510 can be configured to illuminate the at least a portion of sequestration pens with a plurality of illumination spots. The objective 570 can receive the plurality of collimated light beams from the first tube lens 562 and project the plurality of illumination spots, which may form an illumination pattern, into each of the plurality of sequestration pens in the sample plane 574 within the field of view. For example, each of the plurality of illumination spots can have a size of about 5 microns×5 microns; 10 microns×10 microns; 10 microns×30 microns, 30 microns×60 microns, 40 microns X 40 microns, 40 microns×60 microns, 60 microns×120 microns, 80 microns×100 microns, 100 microns×140 microns and any values there between. The illumination spots may individually have a shape that is circular, square, or rectangular. Alternatively, the illumination spots may be grouped within a plurality of illumination spots (e.g., an illumination pattern) to form a larger polygonal shape such as a rectangle, square, or wedge shape. The illumination pattern may enclose (e.g., surround) an unilluminated space that may be square, rectangular or polygonal. For example, each of the plurality of illumination spots can have an area of about 150 to about 3000, about 4000 to about 10000, or 5000 to about 15000 square microns. An illumination pattern may have an area of about 1000 to about 8000, about 4000 to about 10000, 7000 to about 20000, 8000 to about 22000, 10000 to about 25000 square microns and any values there between.

The optical system 510 may be used to determine how to reposition micro-objects and into and out of the sequestration pens of the microfluidic device, as well as to count the number of micro-objects present within the microfluidic circuit of the device. Further details of repositioning and counting micro-objects are found in U. S. Application Publication No. 2016/0160259 (Du); U.S. Pat. No. 9,996,920 (Du et al.); and International Application Publication No. WO2017/102748 (Kim, et al.). The optical system 510 may also be employed in assay methods to determine concentrations of reagents/assay products, and further details are found in U.S. Pat. No. 8,921,055 (Chapman), U.S. Pat. No. 10,010,882 (White et al.), and U.S. Pat. No. 9,889,445 (Chapman et al.); International Application Publication No. WO2017/181135 (Lionberger, et al.); and International Application Serial No. PCT/US2018/055918 (Lionberger, et al.). Further details of the features of optical apparatuses suitable for use within a system for observing and manipulating micro-objects within a microfluidic device, as described herein, may be found in WO2018/102747 (Lundquist, et al), the disclosure of which is herein incorporated by reference in its entirety.

Additional system components for maintenance of viability of cells within the sequestration pens of the microfluidic device. In order to promote growth and/or expansion of cell populations, environmental conditions conducive to maintaining functional cells may be provided by additional components of the system. For example, such additional components can provide nutrients, cell growth signaling species, pH modulation, gas exchange, temperature control, and removal of waste products from cells.

### EXPERIMENTAL

System and device: An OPTOSELECT® device, a nanofluidic device controlled by a BEACON® optical instrument were employed (Both are manufactured by PhenomeX Inc.) The instrument includes: a mounting stage for the chip coupled to a temperature controller; a pump and fluid medium conditioning component; and an optical train including a camera and a structured light source suitable for activating phototransistors within the chip. The OPTOSELECT® device includes a substrate configured with OptoElectroPositioning (OEP®) technology, which provides a phototransistor-activated OET force. The chip also included a plurality of microfluidic channels, each having a plurality of NANOPEN® chambers (or chambers) fluidically connected thereto. The volume of each chamber is around 1×106 cubic microns.

Device priming. 250 microliters of 100% carbon dioxide was flowed in to the OPTOSELECT® device at a rate of 12 microliters/sec, followed by 250 microliters of PBS containing 0.1% PLURONIC® F27 (LIFE TECHNOLOGIES® Cat #P6866) flowed in at 12 microliters/sec, and finally 250 microliters of PBS flowed in at 12 microliters/sec. Introduction of a Wetting Solution follows, which introduces a conditioned surface to the surfaces within the microfluidic device. The details of the surface and its introduction are described in US Application Publication US 2016/0312165, filed on Apr. 22, 2016 and U.S. Application Publication No. US2019/0275516, filed on Nov. 20, 2018, each of which disclosures are herein incorporated by reference in its entirety.

Media perfusion during culture. Medium is perfused through the OPTOSELECT® device according to either of the following two methods: (1) Perfuse at 0.01 microliters/sec for 2 h; perfuse at 2 microliters/sec for 64 sec; and repeat. (2) Perfuse at 0.02 microliters/sec for 100 sec; stop flow 500 sec; perfuse at 2 microliters/sec for 64 sec; and repeat.

System Preparation. OPTOSELECT® chips were loaded onto Beacon instrument for a sequence of pre-workflow operations. The Wetting Solution was introduced to the chip and then incubated to functionalize the surfaces. DI water was then flushed through each chip to remove the Wetting Solution. After wetting and priming, the Beacon instrument automatically located the fiducial markers on the chips for x-y stage and focus calibration. Reference imaging was then performed.

Single-Cell Loading. Cells were loaded in a manner as previously described in U.S. Pat. No. 11,170,200 (Kim et al.), filed on May 31, 2019 and granted Nov. 9, 2021, and U.S. Publication No. US2021/0209752 (Tenney et al.), filed on Nov. 24, 2020. Single cells were identified automatically by the Beacon instrument control software using a convolutional neural network algorithm. A positioning strategy was then automatically implemented to maximize loading throughput using the OEP technology. Residual cells in the channels were flushed to waste after loading.

### Example 1: Displacement of Cells

Cells from a cell population were suspended in PBS and introduced into an OPTOSELECT® microfluidic device, e.g., chip, in a BEACON® Optofluidic system. Single cells were disposed into respective sequestration pens and cultured on-chip. Cells were maintained for a period of time, enabling growth and cells/colonies of interest were identified. Cells within the sequestration pen were moved from a region most distal from the opening of the sequestration pen to a region nearer the pen opening using a suitable method (e.g., OEP® technologies, etc.), creating a fluidic space between the cells and the distalmost portion of the pen.

A flowable hydrogel polymer was introduced in solution, and allowed to diffuse into the sequestration pens. Photoinitiator was also included within the solution containing the flowable hydrogel polymer. The hydrogel polymer was solidified by photopatterning, e.g., photoactivation of polymerization (1 to 1.5 or 3.5 to 5 second exposure, 10× objective, 50% power) to form the solidified hydrogel barriers. In situ-generated hydrogel barriers were formed as pistons, and optionally, guide elements were also formed within a region proximal to the opening of the chamber. In situ-generated pistons were used in cell displacement. The formed piston defined the sequestration pen into two areas (regions). The region that was distal from the opening was a target region (displacement force generation region) and substantially absent of any cell (e.g., a minority or no cells) therewithin. The region proximal to the opening contained a plurality of cells (e.g., a majority, substantially all, or all of the cells) therewithin, and was a cell culture region suitable for cell culture prior to unpenning. A laser illumination was directed to the target region, and a displacement force sufficient to unpen the cells was generated. Cells were unpenned and entered the microfluidic channel. Cells were then exported from the microfluidic device by flowing media within the microfluidic channel.

As shown graphically in FIG. 6A and pictorially in FIG. 6B, selected pens were prepared in order to facilitate unpenning of cells. A timelapse sequence of photographs illustrating the unpenning process is shown in FIG. 7. Starting unpenning of cells, a light actuated dielectrophoretic bar was programmed to move towards the opening of the pen to the channel. The light bar was moved to a location closer to the opening of the pens, having activated the transistors within the substrate, thus moving the cells away from the distal end of the sequestration pen. Following movement of the cells away from the distal end of the sequestration pen, a hydrogel piston was formed as described herein.

Laser illumination was then directed towards an area of the substrate within the target region, e.g., a displacement force generation region, as shown in FIG. 7. The dielectrophoretic force had already cleared cells from that portion of the pen, so direct impact of laser illumination on cells was prevented. The laser illumination induced heating and/or bubble formation in the media displaced the fluid in the target region. The heating (including expansion of the media) and/or bubble exerted a force towards the proximal end of the pen, e.g., towards the opening of the pen, and therefore exerted a force upon the in situ-generated piston, resulting in dissolvement of, displacement of, and/or deformation of the piston. The positioning of the piston between the cells and the target region shielded the cells from forces associated with the displacement force generation, improving viability of the displaced cells and efficiency of displacing cells for unpenning and export.

1. A method for displacing a micro-object from a chamber of a microfluidic device, the method comprising:
providing the microfluidic device, wherein the microfluidic device comprises a microfluidic circuit comprising a substrate, a flow region and the chamber, wherein the chamber comprises an opening to the flow region; wherein the micro-object is disposed within the chamber;
forming an in situ-generated piston within a region of the chamber distal to the micro-object, wherein the in situ-generated piston defines a target region within the chamber distal to the in situ-generated piston; and
illuminating the target region to generate a displacement force thereby displacing the micro-object from the chamber.

2. The method of claim 1, wherein the micro-object further comprises a plurality of micro-objects.

3. The method of claim 2, before forming the in situ-generated piston, the method further comprises moving a portion of the plurality of micro-objects away from a region of the chamber most distal from the opening of the chamber.

4. The method of claim 3, wherein moving a portion of the plurality of micro-objects further comprises moving the portion of the plurality of micro-objects toward the opening of the chamber.

5. The method of claim 1, wherein the target region defined by the in situ-generated piston is substantially absent of the micro-object.

6. The method of claim 1, wherein providing the microfluidic device further comprises disposing the micro-object within the chamber.

7. The method of claim 6, further comprising culturing the micro-object within the chamber.

8. The method of claim 1, wherein illuminating the target region comprises illuminating the target region with a laser.

9. The method of claim 1, wherein illuminating the target region results in generating a bubble within the target region, wherein the bubble generates the displacement force.

10. The method of claim 1, wherein the displacement force pushes the in situ-generated piston away from its original position.

11. The method of claim 10, wherein the displacement force pushes the in situ-generated piston toward the opening of the chamber.

12. The method of claim 1, wherein illuminating the target region comprises directing illumination towards the substrate; a microfluidic circuit material of a wall; or a thermal target.

13. The method of claim 12, wherein the thermal target comprises a metal deposit, a pattern of metal deposits, or microstructures patterned on a surface.

14. The method of claim 1, wherein illuminating the target region comprises illuminating with illumination having incident power in the range of about 1 mW to about 1000 mW.

15. The method of claim 1, wherein the in situ-generated piston has a porosity that substantially prevents the micro-object from crossing through the in situ-generated piston.

16. The method of claim 1, wherein displacing the micro-object comprises exporting the micro-object into the flow region, and optionally, exporting the micro-object from the microfluidic device.

17. The method of claim 16, wherein exporting the micro-object from the microfluidic device further comprises flowing media within the flow region.

18. The method of claim 1, wherein the flow region comprises a microfluidic channel, and the opening of the chamber is proximal to the microfluidic channel and oriented substantially parallel to a direction of flow of a fluidic medium in the microfluidic channel (e.g., when the fluidic medium is flowing in the microfluidic channel).

19. The method of claim 1, wherein the chamber comprises an isolation region and a connection region fluidically connecting the isolation region to the flow region; and wherein the connection region comprises the opening to the flow region.

20. The method of claim 19, wherein the micro-object is disposed within the isolation region.

21-78. (canceled)
